(19) United States
(12) Patent Application Publication
(10) Pub. No.: US 2026/0186942 A1
(43) Pub. Date: Jul. 2, 2026

(54) Artificial Intelligence System for Operation Management

(71) Applicant: Adonis Technology, Inc., New York, NY (US)

(72) Inventors: Aman Magoon, New York, NY (US); Jason Logsdon, New York, NY (US); Shyangmin Chang, New York, NY (US); Nathan Kane, New York, NY (US); Eswar Sriram, New York, NY (US); Daniel Young, New York, NY (US); Akash Magoon, New York, NY (US)

(21) Appl. No.: 19/007,389

(22) Filed: Dec. 31, 2024

(51) Int. Cl. G06F 11/34; G06F 9/451

(52) U.S. Cl. CPC G06F 11/3447; G06F 9/451

(57) ABSTRACT

Implementations include obtaining a first dataset associated with a negative operation cycle event type; determining, via a first set of machine learning components, a set of causation identifiers corresponding to the negative operation cycle event; determining, via a machine learning classification component, a set of classifications comprising a classification of each causation identifier of the set of causation identifiers; training a second set of machine learning components to classify negative operation cycle events having the negative operation cycle event type according to the set of classifications; determining, based on a second dataset, an event classification associated with a negative operation cycle event; determining at least one rectification operation parameter associated with a rectification operation corresponding to the negative operation cycle event; and outputting assignment content configured to cause a user interface of a user device to present a user interface element associated with the rectification operation.

### FIELD

The present disclosure relates to artificial intelligence systems, and more particularly to an artificial intelligence system for operation management.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a block diagram of an example of an artificial intelligence (AI) system.

FIG. 1B is a block schematic diagram associated with processing data from disparate data sources.

FIG. 1C is a block schematic diagram associated with processing data to provide intelligent informational services.

FIG. 1D is a block diagram of another example of an AI system.

FIG. 2 is a block diagram of a computing device.

FIG. 3 is a block schematic diagram associated with machine learning.

FIG. 4 is a block schematic diagram of an example associated with processing data to provide intelligent informational services.

FIG. 5 is a block diagram of another example of an AI system for providing intelligent informational services associated with healthcare workflows.

FIG. 6 is a flow diagram associated with processing data from disparate data sources.

FIG. 7 is a flow diagram associated with providing alerts associated with healthcare workflows.

FIG. 8 is a diagram depicting an example of a graphical user interface associated with providing alerts associated with healthcare workflows.

FIG. 9 is a diagram depicting another example of a graphical user interface (GUI) associated with providing alerts associated with healthcare workflows.

FIG. 10A illustrates a block diagram of an AI system for processing healthcare revenue cycle data.

FIG. 10B illustrates a block diagram of a system architecture for processing healthcare revenue cycle data.

FIG. 11 illustrates a block diagram of an AI system for processing healthcare claims data.

FIG. 12 is a flow diagram of an example of a process for handling predicted events and user feedback.

FIG. 13 is a flow diagram of an example of a process for generating and providing mitigation information.

FIGS. 14A and 14B illustrate interfaces of a healthcare claims processing system.

FIG. 15 illustrates a block diagram of an AI system for processing and managing medical billing information.

FIG. 16 is a flow diagram of an example of a process for assigning and tracking medical billing claims.

FIG. 17 illustrates a GUI for displaying and managing claim worklists.

FIG. 18 is a flowchart of an example of a technique associated with providing alerts associated with healthcare financial workflows.

FIG. 19 is a flowchart of an example of a technique associated with data integration and transformation for healthcare revenue cycle management.

FIG. 20 is a flowchart of an example of a technique associated with data integration and transformation for healthcare revenue cycle management.

FIG. 21 is a flowchart of an example of a technique associated with generating and using predictive models for revenue cycle metrics.

FIG. 22 is a flowchart of an example of a technique associated with processing negative revenue cycle events in healthcare financial workflows.

### DETAILED DESCRIPTION

Various aspects of the disclosure are described more fully hereinafter with reference to the accompanying drawings. This disclosure may, however, be embodied in many different forms and are not to be construed as limited to any specific structure or function presented throughout this disclosure. Rather, these aspects are provided so that this disclosure will be thorough and complete, and will fully convey the scope of the disclosure to those skilled in the art. One skilled in the art may appreciate that the scope of the disclosure is intended to cover any aspect of the disclosure disclosed herein, whether implemented independently of or combined with any other aspect of the disclosure. For example, an apparatus may be implemented or a method may be practiced using any quantity of the aspects set forth herein. In addition, the scope of the disclosure is intended to cover such an apparatus or method which is practiced using other structure, functionality, or structure and functionality in addition to or other than the various aspects of the disclosure set forth herein. Any aspect of the disclosure disclosed herein may be embodied by one or more elements of a claim.

Aspects and examples generally include a method, apparatus, network node, system, computer program product, non-transitory computer-readable medium, computing device, and/or processing system as described or substantially described herein with reference to and as illustrated by the drawings and specification.

This disclosure may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages, are better understood from the following description when considered in connection with the accompanying figures. Each of the figures is provided for the purposes of illustration and description, and not as a definition of the limits of the claims.

While aspects are described in the present disclosure by illustration to some examples, such aspects may be implemented in many different arrangements and scenarios. Techniques described herein may be implemented using different platform types, devices, systems, shapes, sizes, and/or packaging arrangements. For example, some aspects may be implemented via integrated chip embodiments or other non-module-component-based devices (e.g., end-user devices, communication devices, computing devices, industrial equipment, retail/purchasing devices, medical devices, and/or artificial intelligence devices). Aspects may be implemented in chip-level components, modular components, non-modular components, non-chip-level components, device-level components, and/or system-level components. Devices incorporating described aspects and features may include additional components and features for implementation and practice of claimed and described aspects. For example, transmission and reception of signals or data may include one or more components for analog and digital purposes (e.g., hardware components including antennas, radio frequency (RF) chains, power amplifiers, modulators, buffers, processors, interleavers, adders, and/or summers). Aspects described herein may be practiced in a wide variety of devices, components, systems, distributed arrangements, and/or end-user devices of varying size, shape, and constitution.

Several aspects of an artificial intelligence (AI) system will now be presented with reference to various apparatuses and techniques. These apparatuses and techniques will be described in the following detailed description and illustrated in the accompanying drawings by various blocks, modules, components, circuits, steps, processes, or algorithms (collectively referred to as “elements”). These elements may be implemented using hardware, software, or a combination of hardware and software. Whether such elements are implemented as hardware or software depends upon the particular application and design constraints imposed on the overall system.

Several aspects of an artificial intelligence (AI) system will now be presented with reference to various apparatuses and techniques. These apparatuses and techniques will be described in the following detailed description and illustrated in the accompanying drawings by various blocks, modules, components, circuits, steps, processes, or algorithms (collectively referred to as “elements”). These elements may be implemented using hardware, software, or a combination of hardware and software. Whether such elements are implemented as hardware or software depends upon the particular application and design constraints imposed on the overall system.

Data from disparate sources in healthcare operation cycle management (e.g., revenue cycle management, billing cycle management, or appeal cycle management) presents significant challenges due to the inherent differences in data structure, format, and content across various systems. Each data source may have its own unique schema, data types, and naming conventions, making it difficult to seamlessly integrate and analyze information across platforms. For example, an electronic health record (HER) system may store patient demographic information and clinical data in a format optimized for healthcare delivery, while a practice management system may focus on scheduling and billing data with a different organizational structure. Clearinghouse data may include claim submission and response information in yet another format, while payer portals may provide remittance advice and payment data in their own proprietary structure.

Merging this diverse data is complicated by several factors. Inconsistencies in data quality, completeness, and accuracy across sources can lead to conflicts and discrepancies that need to be resolved. Temporal misalignments may occur when different systems update information at varying frequencies or with different time stamps. In some implementations, the semantic interpretation of data fields may vary between systems, requiring careful mapping and transformation to ensure consistent meaning across the unified dataset.

The traditional approach to operation cycle management often involves manual processes and rule-based systems that struggle to keep pace with the dynamic nature of healthcare billing. These systems are limited in their ability to detect subtle changes in payer adjudication practices, leading to increased claim denials and delayed payments. Furthermore, the sheer volume of claims processed by healthcare organizations makes it challenging for human operators to efficiently prioritize and manage workloads, resulting in suboptimal resource allocation and missed opportunities for revenue capture. The lack of real-time insights and predictive capabilities in existing solutions leaves healthcare providers reactive rather than proactive in addressing operation cycle issues, potentially costing them millions of dollars in uncollected revenue annually.

Another significant challenge in the current healthcare operation cycle landscape is the difficulty in leveraging data across multiple provider groups to gain broader insights and improve overall system performance. Existing solutions typically operate in isolation, focusing on individual healthcare organizations without the ability to harness network effects or shared learning across the industry. This limitation prevents healthcare providers from benefiting from the collective intelligence that could be derived from analyzing patterns and trends across a wider dataset. In some implementations, the absence of sophisticated machine learning and artificial intelligence capabilities in many current systems means that healthcare organizations are unable to fully capitalize on the wealth of data at their disposal to drive continuous improvement in their operation cycle processes.

In some specific cases, medical providers face challenges in efficiently processing and appealing denied claims. The complexity of medical billing, coupled with the vast array of payer policies and constantly evolving regulations, creates a landscape where claim denials are frequent and may be difficult to resolve. Traditional methods of managing these denials may rely on manual processes, spreadsheets, and rule-based systems for work assignment. This approach may lead to inefficient resource allocation, delays in accounts receivable, decreased overall revenue, and may contribute to poor employee engagement and high attrition rates among billing staff.

Current systems for managing denied claims may have certain shortcomings. Work assignment is typically based on simplistic rules, such as assigning all claims from a particular payer to a single biller, regardless of the specific actions required for resolution. This lack of granularity in task allocation may result in mismatched skill sets, where billers may be working on claims that do not align with their expertise. Furthermore, the prioritization of work is often based on rudimentary factors like claim age or dollar value, potentially overlooking more nuanced considerations such as the likelihood of successful appeal or the strategic importance of certain payers or claim types. Another challenge lies in the tracking and management of claim statuses throughout the appeal process. Many existing systems may lack robust functionality for differentiating between complete and incomplete tasks, or for providing timely reminders when actions are due. This may lead to claims approaching timely filing deadlines not due to lack of bandwidth, but because of inadequate task management.

Implementations of this disclosure address problems such as these by providing an AI system configured to ingest data of disparate formats, automatically transform that data into a unified format, and use the transformed data to provide intelligent alerts and workflow automations. For example, the AI system may automatically detect the computer storage format of incoming data and dynamically apply appropriate parsing algorithms. This may involve real-time analysis of data structures, encoding schemes, and metadata to determine the most suitable processing approach. The AI system may perform data transformations that may involve multi-step pipelines that normalize, cleanse, and convert the data into a unified format optimized for machine learning and analytics. These transformations may include operations such as data type conversions, unit standardization, code set mapping, and semantic reconciliation across different terminologies. In some implementations, the AI system may utilize parallel processing techniques to handle large volumes of data efficiently, distributing computational tasks across multiple computing devices.

The AI system described herein may employ a flexible and scalable architecture that enables it to ingest, process, and analyze vast amounts of heterogeneous data from multiple sources across the healthcare operation cycle ecosystem. Unlike conventional AI systems that often rely on predefined data models and rigid feature sets, the AI system described herein may utilize advanced machine learning techniques, such as deep neural networks and ensemble methods, to automatically discover complex patterns and relationships within the data. The system's ability to perform unsupervised learning and feature extraction may allow it to identify latent variables and correlations that human experts or traditional rule-based systems might overlook. By leveraging techniques such as transfer learning and multi-task learning, the AI system may efficiently adapt to new data sources and domains without requiring human-directed retraining. This adaptability may enable the AI system to continuously expand its knowledge base and improve its predictive capabilities as it encounters new data patterns and scenarios. The AI system may also incorporate a hierarchical attention mechanism that can dynamically focus on relevant data elements across different timescales and granularities, potentially allowing it to detect subtle anomalies and predict future events based on a holistic analysis of the entire operation cycle ecosystem.

Some implementations may involve an AI system that performs data profiling, schema matching, and data transformation operations on data from multiple sources. The AI system may use an automated machine learning (ML)-based data profiling tool to analyze data from different sources and generate data profile information identifying schema information for each source. An ML component may perform a schema matching operation to identify matches between schemas from different sources.

Based on the schema matching results, an ML-based data transformation component may convert source data into a unified format aligned with a standardized data schema. This transformation process may involve complex operations such as standardizing medical code formats, resolving inconsistencies in units of measurement, and harmonizing semantic interpretations across different systems. The AI system may also incorporate advanced entity resolution techniques, using probabilistic matching algorithms to link patient records or claims data across disparate systems, even in the absence of perfect identifier matches. In some implementations, the system may employ ML-powered data cleaning and completion tools to address data quality issues, correct anomalies, and infer missing information based on historical trends or similarity analyses.

The transformed and unified data may be stored in a database optimized for healthcare operation cycle analytics. An ML-based data management component may oversee data consistency, versioning, and quality within this database, ensuring the integrity and reliability of the unified dataset. The system may also implement AI-powered application programming interfaces (APIs) to facilitate ongoing data flows between the AI system and various data sources, enabling real-time updates and synchronization. This comprehensive approach to data integration and transformation may provide healthcare organizations with a robust foundation for advanced analytics, predictive modeling, and automated decision-making in operation cycle management, addressing the technical limitations of traditional, siloed systems.

The AI system may train and retrain ML models to identify patterns, recognize trends, and detect anomalies. The ML models may be applied to new or real-time data to generate insights, predictions, and actionable decisions. For example, ML applications in healthcare billing include fraud detection and improving revenue cycle management (RCM) processes by enhancing claim submissions and preventing denials. For instance, Natural Language Processing (NLP) may enable the AI system to understand and interpret human language within healthcare workflows. By analyzing textual data such as claim codes, denial messages, and documentation, NLP can streamline tasks like categorizing denial reasons and identifying root causes of errors. This improves efficiency in addressing billing issues and reduces manual intervention.

The AI system may employ various types of models to optimize workflows. Supervised learning models are trained on labeled healthcare billing data, such as claims with known outcomes, to predict results like denial probabilities or claim accuracy. For example, supervised learning may improve claim submissions by learning patterns that lead to successful claims and identifying fields that contribute to denials. In contrast, unsupervised learning models may be used to analyze unlabeled data to detect patterns, groupings, or anomalies. These models can identify unexpected trends in remittance advice or cluster claims by similarities to optimize workflows and detect outliers. Reinforcement learning models may optimize decision-making through a process of interaction and feedback. The AI system may learn from actions taken within the billing workflow and adjust strategies based on positive or negative outcomes, such as improving alert thresholds or prioritizing worklist tasks. Generative AI (GenAI), which includes Large Language Models (LLMs), may enable the AI system to generate new content by learning patterns from existing data. LLMs can summarize claims data, interpret denial codes, and propose resolutions, further enhancing system intelligence.

The AI system's advanced data ingestion and unification capabilities enable it to predict anomalies with unprecedented effectiveness by operating across traditionally siloed data sources and formats. By employing sophisticated machine learning models and natural language processing techniques, the AI system may harmonize diverse data types such as structured claim information, unstructured clinical notes, and semi-structured payer correspondence into a unified data schema. This comprehensive approach allows the AI system to uncover correlations associated with complex, multi-dimensional anomalies that span across different aspects of the revenue cycle. For example, the AI system may identify subtle correlations between specific provider documentation patterns, claim coding practices, and payer-specific adjudication tendencies that consistently lead to denials. In contrast, conventional approaches often struggle to detect such nuanced anomalies and predictions effectively because they are typically limited to analyzing data within individual silos or predefined rule sets. These traditional methods may miss important contextual information and fail to recognize patterns that emerge only when diverse data sources are analyzed holistically. By breaking down these data barriers, the AI system may provide healthcare organizations with a more complete and accurate picture of their revenue cycle performance, enabling them to proactively address issues that were previously invisible or difficult to detect.

In some implementations, the AI system may operate through a three-tier process to identify, predict, and resolve billing issues. First, descriptive analytics may identify problems after they occur by analyzing claim data for anomalies or trends such as patterns of denials. The AI system may generate alerts that highlight discrepancies and provide insights into root causes across the revenue cycle workflow.

In the second tier, predictive analytics may help prevent issues before they arise. For example, predictive denial models may assess claims prior to submission to determine their likelihood of being denied. The AI system may perform pre-claim checks, including patient eligibility verification, provider roster validation, data quality checks (e.g., zip code formatting), and coding assessments for ICD/CPT accuracy, modifiers, and units. By analyzing claims data and identifying patterns, the AI system may return specific reasons why similar claims were denied, allowing organizations to take corrective action before submission. The predictive models also may enable revenue forecasting and provide expected payout estimations for individual claims. Integration with EHR systems may further enhance these capabilities by holding claims for review prior to submission, optimizing claim accuracy, and preventing denials.

For example, implementations of the AI system may involve obtaining a first set of data associated with an entity from a database, generating a machine learning model based on the first set of data, and generating a predictive model by training the machine learning model using at least a portion of the first set of data. The system may then determine a predicted metric associated with a revenue cycle corresponding to the entity based on a second set of data and using the predictive model, and output prediction data indicative of the predicted metric. As used herein, the term “entity” may refer to any healthcare provider organization, such as a hospital, physician group, or clinic. The “first set of data” may include, but is not limited to, patient demographic information, claim information, claim denial information, provider information, or payer information.

The machine learning model used in this system may comprise various types of models, including but not limited to logistic regression models, random forest models, gradient boosting machine models, neural network models, or support vector machine models. Prior to generating the machine learning model, the system may perform pre-processing operations on the structured data, such as data cleaning, feature engineering, or normalization. Feature engineering may involve identifying a set of features associated with the first set of data, which may include claim amount, medical codes, payer type, provider type, patient demographics, or statistics associated with historical metrics corresponding to the predicted metric. Normalization operations may be performed to facilitate consistent feature importance associated with one or more features, which may include encoding categorical data values into numerical data values.

The system may address the challenge of imbalanced data in healthcare billing by implementing various techniques during the training process. These may include using a loss function based on a set of weighted revenue event classes, or performing resampling operations associated with minority revenue event classes. Resampling operations may involve oversampling or undersampling techniques to balance the dataset. The system may also provide flexibility in deployment, allowing for integration with existing healthcare IT infrastructure through API calls, enabling real-time predictions based on incoming data.

Implementations of this disclosure may further address the need for continuous improvement and adaptability by incorporating feedback mechanisms. The system may retrain the predictive model based on feedback information, helping to ensure that the model remains accurate and relevant as patterns in healthcare billing evolve. The predicted metrics generated by the system may include, but are not limited to, predicted medical claim denials, predicted revenue, predicted payouts associated with medical claims, or expected allowed amounts associated with medical claims. These predictions may be presented through user interfaces or used to trigger automated mitigation operations, such as recommendation systems or robotic process automation, to proactively address potential issues in the revenue cycle.

The third tier involves prescriptive analytics, which generates actionable recommendations to resolve billing issues and automate repetitive tasks. For example, the AI system may categorize denial codes, such as Claim Adjustment Reason Codes (CARCs) and Remittance Advice Remark Codes (RARCs), into clear recommendations for resolution. The AI system also may automate corrections, prioritizes worklist assignments, and recommend actions to improve workflows. By capturing feedback on the actions taken, the AI system may continually learn and improve its recommendations.

For example, implementations of the AI system may involve obtaining a dataset associated with a negative revenue cycle event, determining causation information corresponding to the event using machine learning, classifying the causation information, determining rectification operation parameters, and outputting assignment information to present a user interface element associated with the rectification operation. As used herein, a “negative revenue cycle event” refers to any occurrence that negatively impacts the financial processes of a healthcare provider, such as a medical claim denial, delayed payment, or coding error. The term “causation information” includes, but is not limited to, data indicating the root cause or contributing factors leading to the negative revenue cycle event.

The AI system may employ multiple sets of machine learning components to analyze and process the revenue cycle data. A first set of machine learning components, which may include NLP tools, may be used to determine the causation information. This may allow for the extraction of meaningful insights from unstructured data sources such as denial reason descriptions or patient notes. A second set of machine learning components may perform a classification operation to categorize the causation information. This classification may involve supervised learning techniques when labeled data is available, or unsupervised learning methods such as clustering when dealing with unlabeled data.

Based on the classification and using an assignment engine, the system may determine rectification operation parameters associated with addressing the negative revenue cycle event. These parameters may include, but are not limited to, an assigned biller, a priority level, a work status, or a timing parameter. The assignment engine may utilize biller profiles containing information such as biller expertise, workload, and historical performance to optimize task allocation. Priority levels can be determined based on factors including the expected value of resolution, timeliness, and outstanding accounts receivable amounts. The system may then output assignment information configured to present a user interface element associated with the rectification operation on a user device.

The AI system may further incorporate continuous learning and optimization through reinforcement learning techniques applied to various components, including the assignment engine and potentially the automated robotic process automation system. This may allow for ongoing improvement in task allocation and process automation based on historical outcomes and performance data. The AI system may also include pre-processing operations such as biller specialization mapping to enhance the accuracy of work assignments. In some implementations, the AI system can integrate with existing revenue cycle management systems, providing customized dashboards that display not only task assignments but also performance metrics for individual billers or teams.

The AI system may establish a set of data flows associated with a set of data sources, each having a different data schema, and performs a transformation operation using machine learning models to structure the data according to a unified data schema. As used herein, the term “data sources” includes, but is not limited to, EHR systems, practice management systems, clearinghouses, payer portals, and financial systems. The unified data schema may allow for comprehensive analysis across previously siloed data, enabling healthcare providers to gain a holistic view of their revenue cycle. As used herein, the term “unified data schema” refers to a standardized data schema that allows for comprehensive analysis across previously siloed data sources. For example, the unified data schema may include mappings of fields like “patient_id” from an EHR system to “member_number” in a payer portal, enabling cross-system data analysis.

The AI system may perform data integration and normalization from multiple healthcare information technology (IT) systems using probabilistic matching techniques. As used herein, “probabilistic matching” refers to algorithms that determine the likelihood that records from different sources refer to the same entity, even when unique identifiers are not available. For example, the AI system may use a combination of name, date of birth, and address to match patient records across systems with a certain confidence level.

The AI system may perform anomaly detection operations on the structured data using machine learning models to identify anomalies associated with claims, denials, payments, reimbursements, submissions, or payment histories. As used herein, an “anomaly” refers to any unusual pattern, trend, or deviation from expected behavior in the revenue cycle data. The AI system may generate alert events based on these anomalies and output alert data to user devices based on customized alert profiles. The term “alert profile” includes, but is not limited to, user-specific settings that determine which types of alerts are displayed and how they are prioritized. This approach may enable healthcare providers to proactively address revenue cycle issues and optimize their financial performance.

For example, the AI system may generate alerts to highlight discrepancies and provide insights into root causes across the revenue cycle workflow. As used herein, an “alert” (referred to herein, interchangeably as an “alert event”) refers to a notification of an anomaly or issue, which may be delivered through various channels such as email, SMS, or in-app notifications. The AI system may output alert data configured to cause a user interface of a user device to present a user interface element associated with the alert event. This user interface element may include a selectable option configured to cause the user interface to present information associated with the anomaly. For example, an alert may notify a billing manager of an unusual spike in claim denials for a particular procedure code, with the option to view detailed analytics on the affected claims.

The AI system may incorporate reinforcement learning that uses feedback from human billers to continuously improve its recommendations and automated actions. In some implementations, the AI system employs predictive modeling to forecast claim denials and payment timelines, enabling proactive interventions. For instance, the AI system may predict that a certain type of claim has a high likelihood of denial based on historical patterns and recommend pre-submission review.

The AI system may leverage data across multiple provider groups to harness network effects and shared learning. As used herein, “network effects” refer to the improved performance and accuracy of the system as more healthcare providers contribute data. The AI system may utilize ML models trained on this broader dataset to identify claim structure patterns and generate more accurate anomaly detection models. In some implementations, the AI system may organize metrics into metric groups, with each group associated with a different metric type. The term “metric group” includes, but is not limited to, collections of related financial or operational metrics such as denial rates, accounts receivable aging, or reimbursement rates. By decoupling individual metrics from metric groups, the system may provide flexibility in analyzing and reporting on revenue cycle performance across various dimensions.

The AI system's ability to detect anomalies that were previously undetectable stems from its comprehensive integration and analysis of data from multiple disparate sources. By leveraging ML models trained on a unified data schema, the AI system may identify subtle patterns and correlations that would be impossible to detect when examining each data source in isolation. For example, the AI system may uncover anomalies related to specific combinations of diagnosis codes, procedure codes, and payer policies that consistently lead to claim denials. These complex relationships may not be apparent when looking at claims data, clinical data, or payer data separately, but become visible when analyzed holistically. This enhanced anomaly detection capability may allow healthcare providers to address previously hidden issues in their revenue cycle, potentially reducing denial rates and improving overall financial performance.

The proactive nature of the AI system's anomaly detection is achieved through its real-time data processing and predictive analytics capabilities. Instead of relying on retrospective analysis of historical data, the AI system may continually monitor incoming data streams and apply ML models to identify potential issues before they escalate. For instance, the AI system may detect a slight increase in the time between claim submission and payment for a particular payer, which could indicate a change in their adjudication process. By alerting healthcare providers to this trend early, the AI system may enable them to investigate and address the issue proactively, potentially preventing a more significant disruption to their cash flow. This proactive approach may lead to faster resolution of revenue cycle issues, improved operational efficiency, and a more stable financial position for healthcare organizations.

The benefits of these advanced anomaly detection capabilities may be multifaceted. By identifying previously undetectable anomalies, healthcare providers may uncover new opportunities for process improvement and revenue optimization. This may lead to reduced revenue leakage, improved claim acceptance rates, and more accurate forecasting of cash flows. The proactive nature of the system's anomaly detection may result in faster issue resolution, minimized financial impact of revenue cycle disruptions, and improved resource allocation. In some implementations, the AI system's ability to learn and adapt over time means that its anomaly detection capabilities may continually improve, providing healthcare organizations with an increasingly powerful tool for managing their revenue cycle and staying ahead of emerging challenges in the complex healthcare billing landscape.

The disclosed technology relates to a system and method for addressing challenges in healthcare revenue cycle management by employing ML and AI to aggregate, normalize, and analyze clinical and billing data across disparate systems. These systems may include EHRs, practice management systems, clearinghouses, payer portals, and financial management platforms. The described implementation focuses on integrating data from non-standardized sources into a unified format and using ML to optimize workflows associated with claims processing, denial management, and operational efficiency.

The system may process datasets associated with negative revenue cycle events, such as medical claim denials, delayed payments, or coding errors. By leveraging AI, the system may determine causation information using natural language processing (NLP) and other ML techniques to extract actionable insights from structured and unstructured data sources. These insights may include identifying root causes of claim denials or anomalies in claim patterns. The causation information may then be classified using supervised or unsupervised learning models to categorize issues by denial type, payer, or other criteria.

The classification data is utilized by an assignment engine configured to allocate tasks, such as rectification operations, based on biller expertise, workload, historical performance, and priority levels. This engine incorporates reinforcement learning to continually optimize task assignments and enhance system performance based on feedback and historical outcomes. Pre-processing operations, including feature engineering and normalization, may ensure consistent data interpretation and integration across systems.

The system also provides predictive analytics capabilities to forecast issues such as claim denials or payment delays before they occur. These capabilities may include pre-submission checks for claim accuracy, including coding validation (e.g., ICD, CPT codes), eligibility verification, and data quality assessments. By analyzing historical patterns and payer-specific requirements, the system can proactively suggest corrective actions to reduce denial rates and improve claim accuracy.

Anomaly detection is another core functionality of the system, enabling the identification of unusual patterns in claims, payments, or operational metrics. The system may generate alerts to notify users of these anomalies, providing actionable insights via user interfaces. For instance, alerts may highlight discrepancies in claim adjudication times or unusual denial rates for specific procedure codes. These notifications may include interactive user interface elements to allow detailed examination of the associated issues.

To address the fragmentation of data sources, the system performs data integration and normalization through probabilistic matching and transformation operations. These processes may unify datasets with differing schemas, facilitating comprehensive analysis and enabling healthcare providers to achieve a holistic view of their revenue cycle. For example, the system may align disparate data fields, such as mapping “patient_id” from EHR systems to “member_number” in payer systems, within a unified schema.

Prescriptive analytics functionalities within the system may further streamline revenue cycle management by providing actionable recommendations for resolving billing issues. For instance, the system may categorize denial codes, such as Claim Adjustment Reason Codes (CARCs), into standardized resolution workflows and automate repetitive tasks, such as correcting claim errors or generating appeal letters.

The AI system is continually updated through feedback loops and reinforcement learning techniques, enabling it to adapt to evolving payer policies and operational requirements. By leveraging shared learning across multiple provider organizations, the system enhances its predictive and prescriptive capabilities, enabling broader insights and optimized decision-making.

These implementations specifically address technological challenges rooted in computing technology, such as transforming heterogeneous datasets into standardized formats and applying advanced ML algorithms for workflow optimization. The system's use of real-time predictive modeling and cross-platform data integration positions it as a tool for addressing complex issues in healthcare billing and revenue management, improving financial performance and operational efficiency for healthcare providers.

These systems and methods may serve to process datasets associated with negative revenue cycle events, identify causative factors using machine learning models, and classify such factors for optimized rectification operations. These functionalities leverage an assignment engine, which may utilize machine learning for resource optimization, such as assigning tasks based on biller expertise, priority levels, and timing parameters. The solutions integrate various pre-processing and post-processing operations, including feature engineering and reinforcement learning, to continually improve task allocations and operational efficiency within revenue cycle management systems.

Thus, these implementations extend beyond mere commercial activities, operations that can be performed mentally, and mere organizations of human activity, as they are rooted in computing technology. Furthermore, the implementations specifically address challenges associated with electronic revenue cycle workflow applications and the software systems that facilitate such workflows over networks, including the Internet.

The AI system may implement robust data privacy and security measures to ensure compliance with data protection regulations. The AI system may employ advanced encryption techniques for data at rest and in transit, utilizing industry-standard protocols to safeguard sensitive patient and financial information. Access controls and user authentication mechanisms may be implemented to ensure that only authorized personnel can view or modify specific data sets. The AI system may also incorporate data anonymization and pseudonymization techniques when processing large datasets for analytics, reducing the risk of individual patient identification. Regular security audits and vulnerability assessments may be conducted to identify and address potential weaknesses in the system's infrastructure. In some implementations, the AI system may include features for data retention and deletion in accordance with legal requirements and organizational policies.

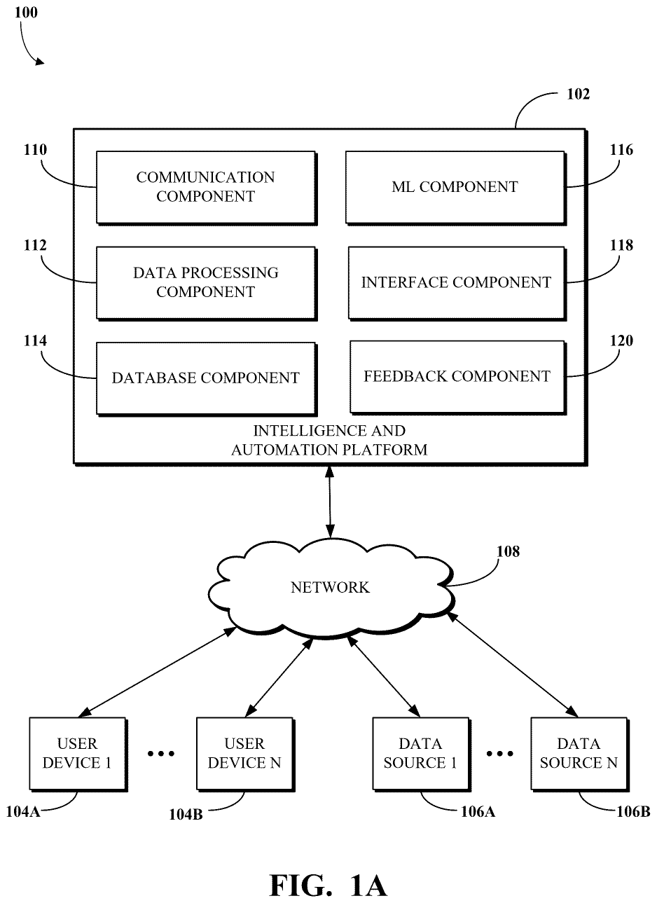

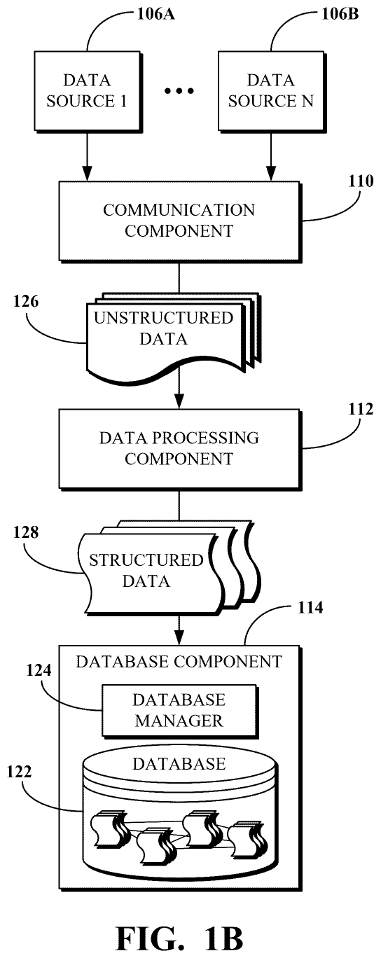

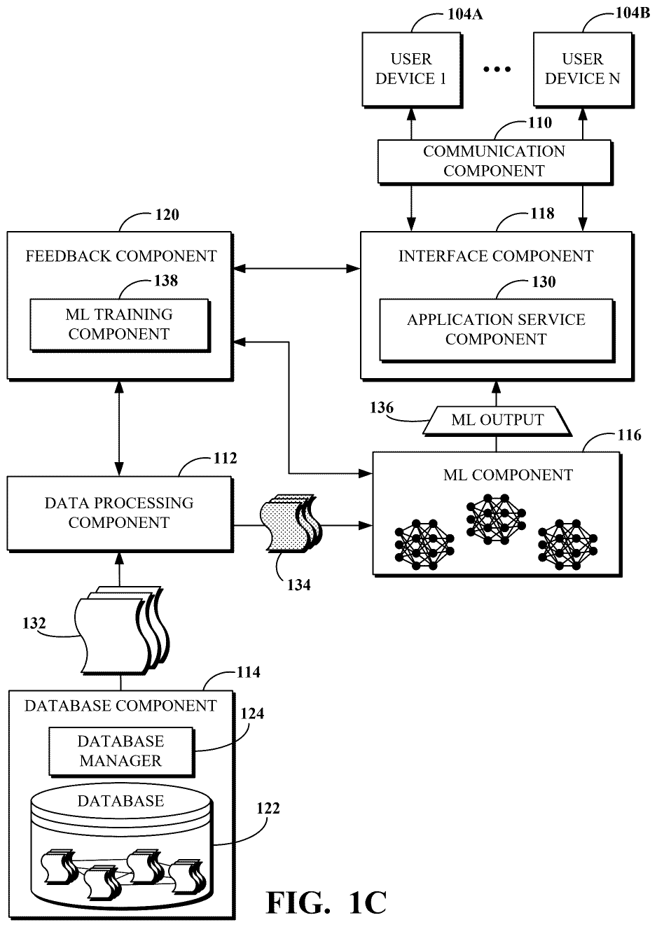

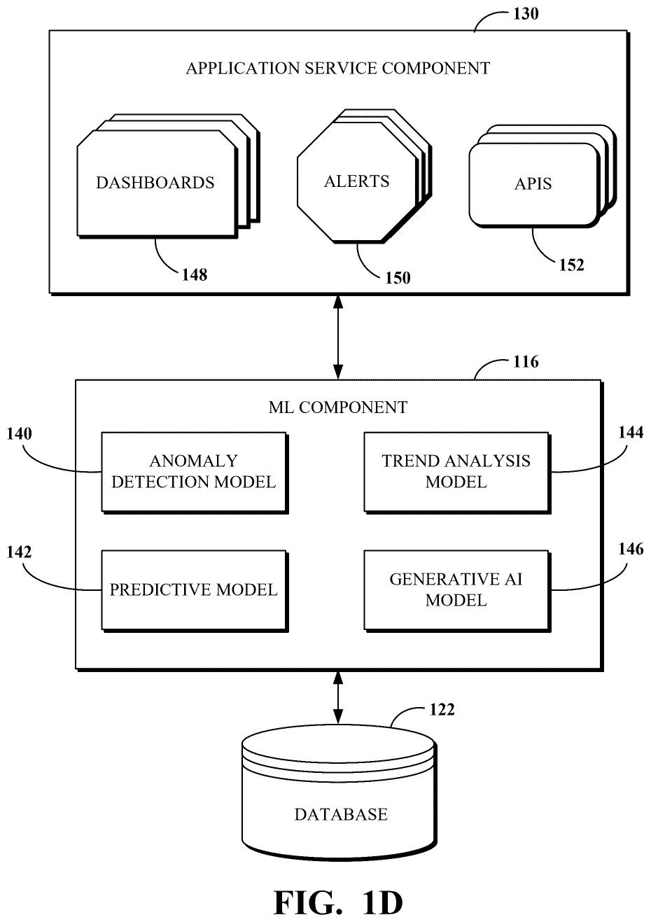

FIG. 1A is a block diagram of an example of an AI system 100, which can be, or include, a distributed computing system, a cloud computing system, and/or a clustered computing system, among other examples. As shown, the AI system 100 includes an intelligence and automation platform 102, user devices 104 (shown as user device 1 104A and user device N 104B), and data sources 106 (shown as data source 1 106A and data source 106B), communicatively coupled by a network 108. The AI system 100 may be implemented using a hardware environment that includes computer system components, such as general-purpose computers, dedicated computer systems, peripheral devices, components, and modules, and/or a combination thereof. In some implementations, the AI system 100 may be implemented within one or more cloud computing environments, where various components of the AI system 100 may be executed in various configurations, including in parallel. In some implementations, one or more components of the AI system 100 can be implemented using one computing device or a combination of several interconnected computing devices.

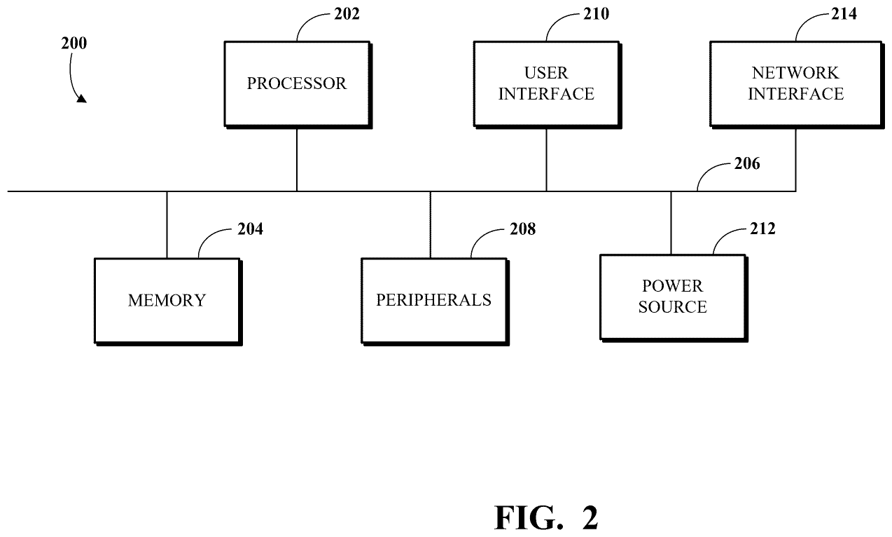

The user devices 104 may include any device that enables a user to interact with the AI system 100. The user devices 104 may include a mobile device, a tablet, a personal computer, a wearable device, or any other computing device. The user devices 104 may operate a suitable operating system, such as a desktop operating system, a mobile operating system, or a web browser. In some implementations, the user devices 104 may be implemented using one or more computing devices such as the computing device 200 illustrated with respect to FIG. 2.

The data sources 106 may include one or more computing devices configured to provide electronic data, electronic files, electronic signatures, electronic documents, or any other electronic data to the AI system 100 or to another aspect of the AI system 100. For example, the data sources 106 may be implemented within one or more cloud computing environments, where various components of the data sources 106 may be executed in various configurations, including in parallel. In some implementations, one or more components of the data sources 106 can be implemented using one computing device or a combination of several interconnected computing device such as the computing device 200 illustrated with respect to FIG. 2. The data sources 106 may include a cloud resource, a workstation, an EHR, a practice management system, a clearinghouse service, a payer portal, and/or a financial system, among other examples of data sources 106.

The network 108 may be a public communication network (e.g., the Internet, cellular data network, dialup connectivity, etc.), a private communications network (e.g., private LAN, leased line, etc.), or a combination of a public communications network and a private communications network. In some cases, the network 108 may include, and/or may communicate with, any one or more of a Bluetooth network, a near-field communication network, a satellite communication network, a wireless communication network, and/or any other communication network. The network 108 may include any combination of wired and/or wireless networks. The communication over the network 108 can traverse one or more of the Internet, a wide area network (WAN), a metropolitan area network (MAN), a local area network (LAN), a virtual private network (VPN), a wireless local area network (WLAN), a virtual private wireless network (VP), a radio access network, a mobile data network, a power distribution network, a satellite network, a plain old telephone system (POTS), and/or a cellular or third generation (3G) or fourth generation (4G) data network, among other examples, and/or any combination of the above.

As shown, the intelligence and automation platform 102 includes a communication component 110, a data processing component 112, a database component 114, an ML component 116, an interface component 118, and a feedback component 120. In some implementations, one or more of the communication component 110, the data processing component 112, the database component 114, the ML component 116, the interface component 118, and the feedback component 120 may be implemented using one computing device or a combination of several interconnected computing device such as the computing device 200 illustrated with respect to FIG. 2. In some implementations, two or more of the communication component 110, the data processing component 112, the database component 114, the ML component 116, the interface component 118, and the feedback component 120 may be combined into a single component.

The intelligence and automation platform 102 may serve as the core of the AI system 100, processing and analyzing healthcare revenue cycle data to provide actionable insights and automate various tasks. The platform 102 may be implemented using cloud computing infrastructure in some implementations, allowing for scalability and flexibility. In some implementations, the platform 102 could be deployed on-premises or in a hybrid cloud configuration, depending on the specific needs and regulatory requirements of healthcare providers.

The communication component 110 may manage the flow of information between the platform and external systems, including user devices 104 and data sources 106. This component may implement various communication protocols and data exchange standards specific to healthcare IT, such as HL7 FHIR or X12 EDI. In some embodiments, the communication component 110 may also support blockchain technology for enhanced data integrity and traceability in healthcare transactions.

The data processing component 112 may be responsible for ingesting, cleaning, and transforming the raw data received from various sources into a standardized format suitable for analysis. This component may employ data integration techniques, including probabilistic matching algorithms, to reconcile inconsistencies across different data sources. The data processing component 112 may utilize parallel processing frameworks, such as Apache Spark, to handle large volumes of healthcare data efficiently. In some implementations, this component may also incorporate NLP capabilities to extract meaningful information from unstructured clinical notes or payer correspondence.

The database component 114 may provide a unified data repository for the AI system 100, storing both raw and processed data in a structured manner. This component may utilize a combination of relational and NoSQL databases to accommodate the diverse types of healthcare data encountered in revenue cycle management. The database component 114 may implement data modeling techniques to represent complex relationships between various entities, such as patients, claims, providers, and payers. In some embodiments, the database component 114 may incorporate a data lake architecture to store and analyze large volumes of unstructured and semi-structured data.

The ML component 116 may be the analytical engine of the AI system 100, leveraging various machine learning algorithms to detect patterns, anomalies, and trends in healthcare revenue cycle data. ML is a subset of AI that focuses on the development of algorithms that allow computers to learn from data inputs and make predictions or decisions without explicit programming. ML leverages large datasets to identify patterns, make decisions, and improve over time based on experience. ML focuses on creating systems that can learn from data, adapt to new inputs, and generate predictions or actions.

For example, an ML component may be or include one or more ML models, ML algorithms, and/or ML systems including combinations of ML algorithms and ML models. An ML component may be implemented on any number of different hardware devices and may include one or more machine learning models. ML is a field of study that gives computers the ability to perform certain tasks without being explicitly programmed to perform those tasks. In traditional computing, a programmer would encode instructions (e.g., to solve a quadratic equation using the quadratic formula), and the computer would perform those exact instructions. In contrast, in ML, a computer can be provided with examples and be trained to perform a task such as prediction or classification, without the programmer encoding explicit instructions for the task. ML explores the study and construction of algorithms, also referred to herein as tools, models, and/or components, which may learn from existing data and make predictions about new data. Such ML tools operate by building a model from example training data in order to make data-driven predictions or decisions expressed as outputs or assessments. Although example embodiments are presented with respect to a few ML models, the principles presented herein may be applied to other ML models. In some example embodiments, different ML models may be used. ML models may include, for example, K-means clustering models, linear regression models, logistic regression (LR) models, Naive-Bayes models, random forest (RF) regression models, gradient boost models, neural networks (NN), matrix factorization models, large language models (LLMs), and/or support vector machines (SVMs), among other examples.

The ML component 116 may employ supervised learning techniques to predict claim denials, unsupervised learning for anomaly detection, and reinforcement learning to optimize billing workflows. The ML component 116 may utilize ensemble methods, combining multiple models to improve accuracy and robustness. In some implementations, the ML component 116 may also incorporate deep learning techniques, such as recurrent neural networks (RNNs), to analyze sequential data in claims processing.

The interface component 118 may provide the user-facing layer of the AI system 100, delivering insights and functionalities through intuitive dashboards, reports, and interactive visualizations. This component may implement responsive design principles to ensure a consistent user experience across various devices and screen sizes. The interface component 118 may utilize data visualization libraries, such as D3.js, to create dynamic and interactive representations of complex revenue cycle metrics. In some embodiments, the interface component 118 could incorporate augmented reality (AR) features to provide immersive data exploration experiences for healthcare finance professionals.

The feedback component 120 may play a role in the continuous improvement of the AI system 100 by capturing user interactions, preferences, and manual corrections. This component may implement mechanisms to collect both explicit feedback (e.g., user ratings) and implicit feedback (e.g., usage patterns) to refine the system's algorithms and recommendations. The feedback component 120 may employ A/B testing frameworks to evaluate the effectiveness of different features or UI elements. In some implementations, this component could utilize sentiment analysis techniques to gauge user satisfaction and identify areas for improvement based on natural language feedback.

FIG. 1B illustrates a block schematic diagram associated with processing data from disparate data sources, highlighting the data flow and transformation processes within the AI system 100. The communication component 110 receives data from multiple sources, including data source 106A and data source 106B, and outputs unstructured data 126. This unstructured data may include a mix of structured (e.g., claims data in standardized formats) and unstructured (e.g., clinical notes, denial reasons) information from various healthcare IT systems.

The data processing component 112 takes the unstructured data 126 as input and performs a series of transformations to generate structured data 128. This process may involve data cleaning, normalization, and enrichment tasks tailored to healthcare revenue cycle management. The data processing component 112 may employ entity resolution techniques to reconcile patient and provider information across different systems, ensuring data consistency and accuracy.

The structured data 128 is then passed to the database component 114, which manages its storage and retrieval. The database component 114 includes a database manager 124 that oversees the organization and indexing of data within the database 122. This architecture may allow for efficient querying and analysis of large-scale healthcare financial data, supporting both real-time operational needs and long-term analytical requirements.

FIG. 1C is a block schematic diagram associated with processing data to provide intelligent informational services. FIG. 1C provides a more detailed view of the data processing and machine learning pipeline within the AI system 100. The system interacts with multiple user devices (104A, 104B) through the communication component 110, which interfaces with the interface component 118. The interface component 118 contains an application service component 130, which manages the business logic and user interactions of the AI system.

The data processing component 112 retrieves data 132 from the database component 114 and generates processed data 134. This processed data serves as input for the ML component 116, which applies various machine learning algorithms to produce ML output 136. The ML output, which may include predictions, anomaly detections, or optimized workflows, is then provided to the application service component 130 for presentation to users.

A feature of the AI system 100 may be its ability to continuously learn and improve. The feedback component 120 contains an ML training component 138 that interacts with both the data processing component 112 and the interface component 118. This feedback loop may enable the system to adapt to changing patterns in healthcare billing and user preferences, enhancing its accuracy and relevance over time.

The ML component 116 may be configured to perform various types of training to enhance its capabilities in healthcare revenue cycle management. In some implementations, the ML component 116 may utilize supervised learning techniques to train models for predicting claim denials, reimbursement amounts, and other metrics. This training process may involve feeding the models large datasets of historical claims data, including features such as patient demographics, diagnosis codes, procedure codes, provider information, and payer details, along with the corresponding outcomes (e.g., approved, denied, partially paid).

To create training datasets, the AI system 100 may aggregate data from multiple healthcare providers across various specialties and geographical regions. This comprehensive dataset may include information from electronic health records (EHRs), practice management systems, clearinghouses, and payer portals. In some cases, the system may employ data augmentation techniques to enhance the quality and quantity of training data. For instance, the AI system 100 may use generative adversarial networks (GANs) to create synthetic medical claims that mimic the characteristics of real claims, helping to balance datasets for rare conditions or uncommon billing scenarios.

The ML component 116 may also implement unsupervised learning algorithms to detect anomalies and patterns in the revenue cycle data without relying on labeled outcomes. These models may be trained on large volumes of unlabeled claims data to identify unusual trends, potential fraud, or emerging issues in payer behavior. The training process for unsupervised models may involve techniques such as clustering, dimensionality reduction, and anomaly detection algorithms like isolation forests or autoencoders.

In some implementations, the ML component 116 may employ reinforcement learning techniques to optimize decision-making processes in revenue cycle management. This training approach may involve creating a simulated environment that mimics the complexities of healthcare billing, where the model learns to make optimal decisions through trial and error. The reinforcement learning models may be trained to maximize revenue capture, minimize denial rates, or optimize resource allocation in billing workflows.

The AI system 100 may implement dynamic retraining mechanisms to ensure that the ML models remain accurate and relevant in the face of changing healthcare regulations and payer policies. One approach to dynamic retraining may involve implementing a sliding window technique, where the system continuously updates the training dataset with the most recent claims data while gradually phasing out older data. This approach may help the models adapt to evolving patterns in claim adjudication and payer behavior.

Another method for dynamic retraining may leverage federated learning techniques, allowing the AI system 100 to learn from data across multiple healthcare providers without directly sharing sensitive patient or claim information. In this approach, model updates may be distributed to individual healthcare providers, who then train the model on their local data and return only the updated model parameters. The system may then aggregate these updates to improve the global model, potentially capturing nuanced patterns that may not be apparent in any single provider's data alone.

The creation of retraining datasets may involve a combination of automated data collection and human-in-the-loop processes. The AI system 100 may automatically gather new claims data, remittance advice, and payer correspondence as they become available. In some implementations, the system may incorporate feedback from human billers and revenue cycle managers, capturing information about successful appeals, coding corrections, and other manual interventions that lead to positive claim outcomes. This feedback may be used to enrich the retraining datasets, helping the models learn from expert knowledge and adapt to complex edge cases.

In some implementations, the ML component 116 may utilize active learning techniques to identify the most informative samples for retraining. This approach may involve selecting claims that the current model is uncertain about or that represent edge cases in the data distribution. For example, if the model consistently misclassifies certain types of claims or struggles with specific payer-provider combinations, these cases may be prioritized in the retraining dataset. The active learning approach may help optimize the retraining process by focusing on the most challenging and informative examples, potentially improving model performance more efficiently than random sampling of new data.

FIG. 1D illustrates the machine learning architecture of the AI system 100 in greater detail. The application service component 130 provides three main interfaces: dashboards 148, alerts 150, and APIs 152. These interfaces may enable users to interact with the system's insights and functionalities in various ways, from visual analytics to programmatic integrations.

The ML component 116 houses multiple specialized models, each addressing specific aspects of healthcare revenue cycle management. The anomaly detection model 140 identifies unusual patterns in claims data, helping to flag potential issues before they impact revenue. The predictive model 142 forecasts outcomes such as claim denials or payment timelines, enabling proactive interventions. The trend analysis model 144 uncovers long-term patterns in revenue cycle performance, supporting strategic decision-making. The generative AI model 146 may be used for tasks such as automated report generation or natural language interactions with the system.

This AI architecture may provide a solution to technical challenges of fragmented and inefficient healthcare revenue cycle management. By integrating data from multiple sources, applying machine learning techniques, and providing actionable insights through intuitive interfaces, the AI system 100 may enable healthcare providers to optimize their financial operations, reduce claim denials, and improve overall revenue capture. The system's ability to learn and adapt may allow it to remain effective in the face of evolving healthcare regulations and payer policies, providing advantages for healthcare organizations that implement this technology.

FIG. 2 is a block diagram of a computing device 200. In some implementations, the computing device 200 may implement one or more of the intelligence and automation platform 102, the user devices 104, and the data sources 106 of the AI system 100 shown in FIG. 1A.

The computing device 200 includes a processor 202, a memory 204, a bus 206, peripherals 208, a user interface 210, a power source 212 and a network interface 214. In some implementations, the computing device 200 may include any number of other components. The bus 206 may facilitate communication between two or more of the processor, the memory 204, the peripherals 208, the user interface 210, the power source 212 and the network interface 214.

The processor 202 (referred to herein interchangeably as “processing circuitry”) may include a central processing unit, such as a microprocessor. The processor 202 may include single or multiple processors having single or multiple processing cores. In some implementations, the processor 202 may include another type of device, or multiple devices, configured for manipulating or processing information. One or more operations of the processor 202 may be distributed across multiple devices or units that may be coupled directly or across a local area network or other suitable type of network. The processor 202 may include a cache, or cache memory, for local storage of operating data or instructions.

The memory 204 may include one or more memory components, each of which may be volatile memory or non-volatile memory. For example, volatile memory may be random access memory (RAM) (e.g., a DRAM module, such as DDR SDRAM). In another example, non-volatile memory may be a disk drive, a solid state drive, flash memory, or phase-change memory. In some implementations, the memory 204 may be distributed across multiple devices. For example, the memory 204 may include network-based memory or memory in multiple clients or servers performing the operations of those multiple devices.

The memory 204 may include data for access by the processor 202. For example, the memory 204 may include executable instructions that may be executed by the processor 202. Reference to execution, by the processor 202, of executable instructions stored in the memory 204 may include a reference to execution by multiple processors of the same or different instructions, which may be stored in, or across, one or more memories. The executable instructions may correspond to one or more application programs, which may be loaded or copied, in whole or in part, from non-volatile memory to volatile memory to be executed by the processor 202. For example, the executable instructions may include instructions for performing some or all of the techniques of this disclosure. The data stored in the memory may include user data, database data (e.g., database catalogs or dictionaries), or the like. In some implementations, the data may include functional programs, such as a web browser, a web server, a database server, another program, or a combination thereof.

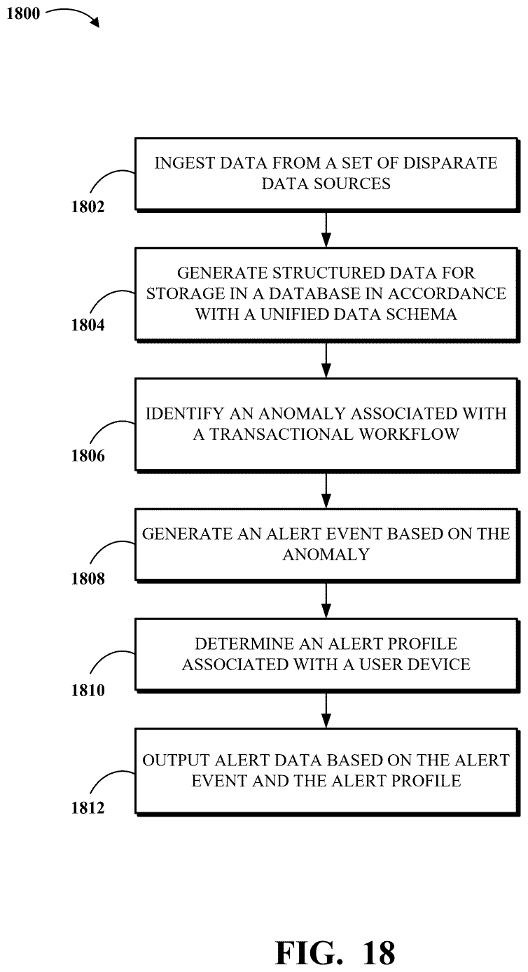

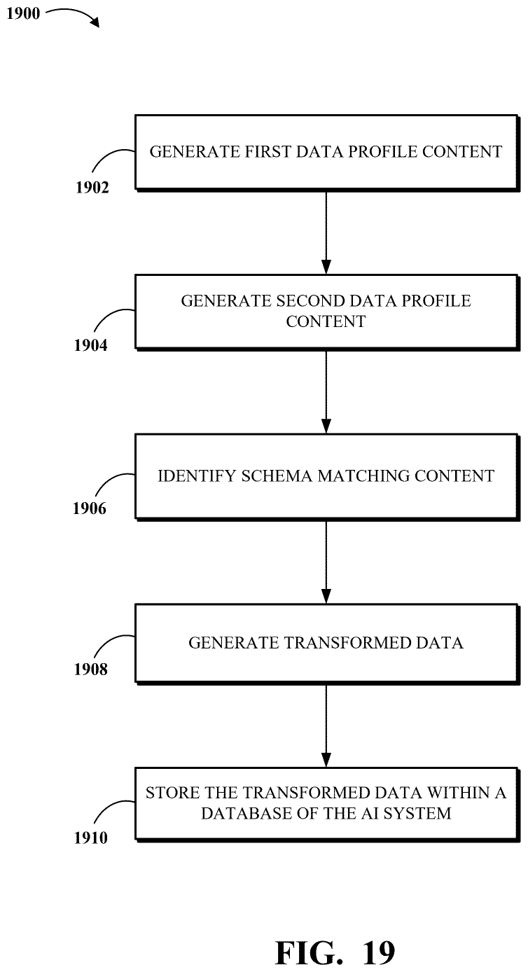

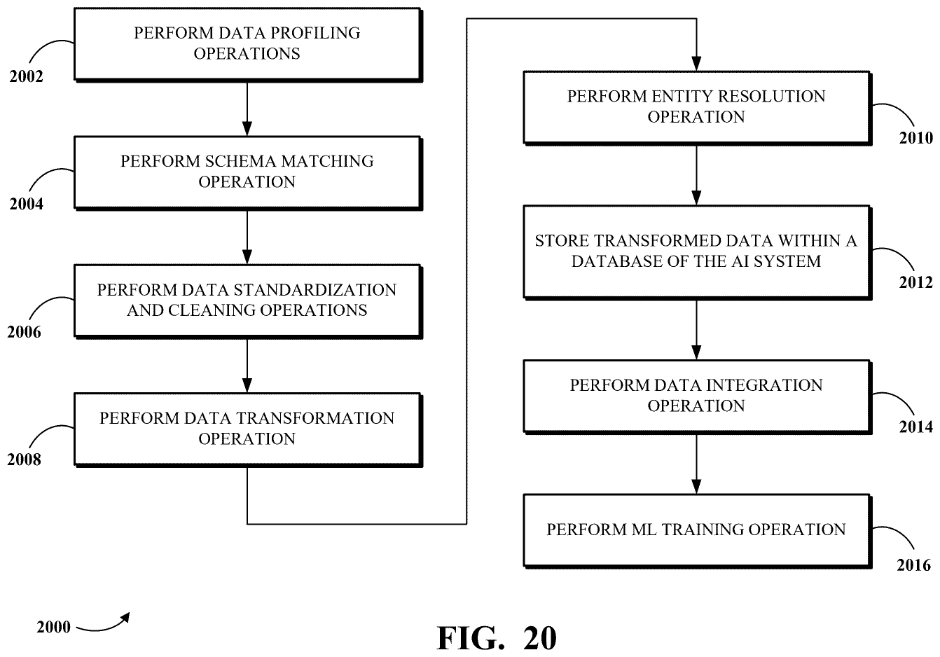

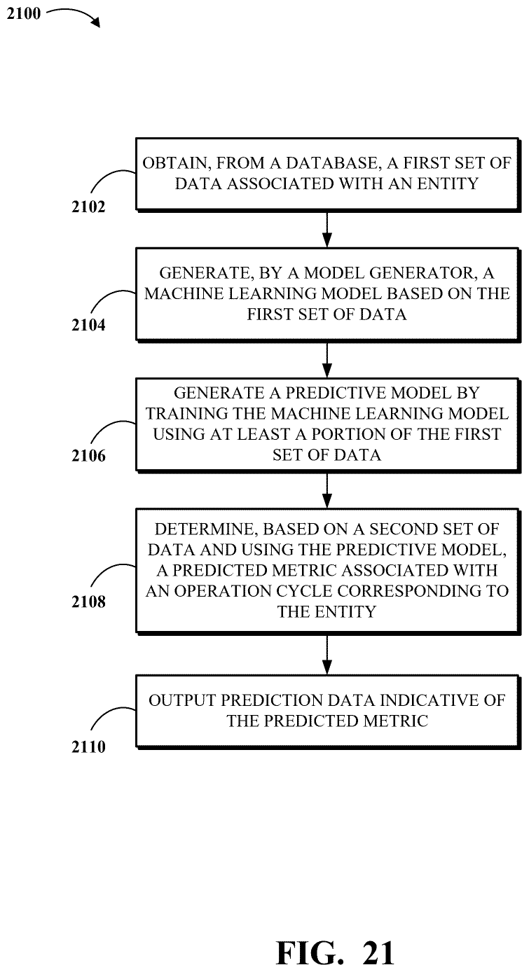

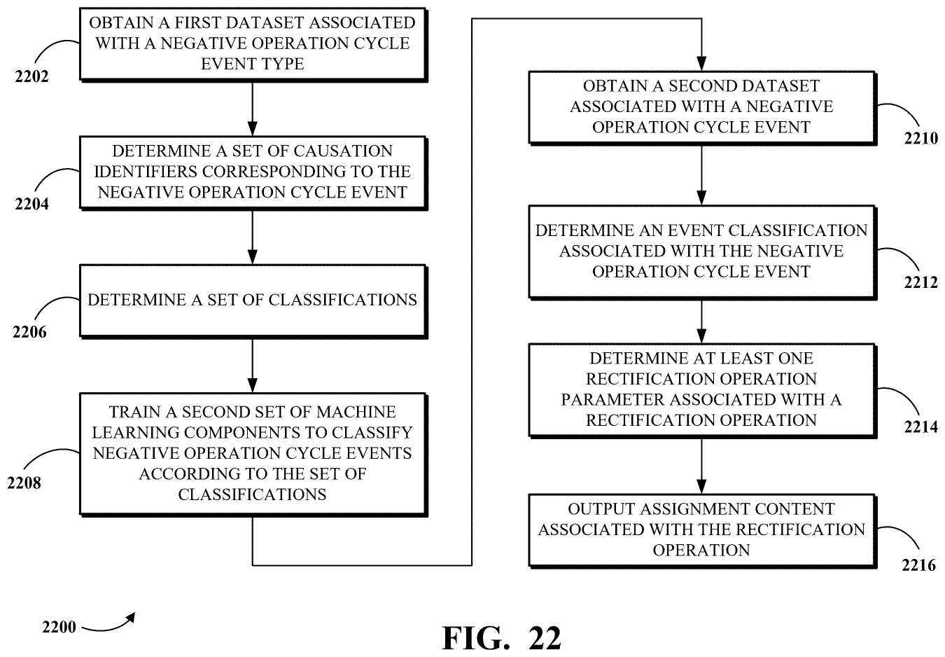

The processor 202 may implement one or more techniques or perform one or more operations associated with machine-learning based clinical workflows, as described in more detail elsewhere herein. For example, the processor 202 may perform or direct operations of, for example, technique 1800 of FIG. 18, technique 1900 of FIG. 19, technique 2000 of FIG. 20, technique 2100 of FIG. 21, technique 2200 of FIG. 22, or other techniques as described herein (alone or in conjunction with one or more other processors). The memory 204 may store data and program codes for the computing device 200. In some examples, the memory 204 may include a non-transitory computer-readable medium storing a set of instructions (for example, code or program code). The memory 204 may include one or more memories, such as a single memory or multiple different memories (of the same type or of different types). For example, the set of instructions, when executed (for example, directly, or after compiling, converting, or interpreting) by the processor 202, may cause the processor to cause the computing device 200 to perform technique 1800 of FIG. 18, technique 1900 of FIG. 19, technique 2000 of FIG. 20, technique 2100 of FIG. 21, technique 2200 of FIG. 22, or other techniques as described herein. In some examples, executing instructions may include running the instructions, converting the instructions, compiling the instructions, and/or interpreting the instructions, among other examples.

The peripherals 208 may include one or more peripheral devices such as, for example, sensors, detectors, or other devices configured for obtaining data associated with the computing device 200, a user of the computing device 200, or the environment around the computing device 200. For example, the peripherals 208 may include a geolocation component, such as a global positioning system location unit. In another example, the peripherals may include a temperature sensor for measuring temperatures of components of the computing device 200, such as the processor 202. In some implementations, the computing device 200 may omit the peripherals 208.

The user interface 210 may include one or more input interfaces and/or output interfaces. An input interface may, for example, include a positional input device, such as a mouse, touchpad, touchscreen, or a keyboard, among other examples. An output interface may, for example, be a display, such as a liquid crystal display, a cathode-ray tube, or a light emitting diode display, among other examples.

The power source 212 may be configured to provide power to the computing device 200. For example, the power source 212 may include an interface to an external power distribution system. In another example, the power source 212 may include a battery. In some implementations, the computing device 200 may include or otherwise use multiple power sources.

The network interface 214 may facilitate communication via a network (e.g., the network 108 shown in FIG. 1A). The network interface 214 may include a wired network interface, a wireless network interface, or a combination thereof. The computing device 200 may communicate with other devices via the network interface 214 using one or more network protocols such as, for example, Ethernet, transmission control protocol (TCP), internet protocol (IP), power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), or Z-Wave, among other examples.

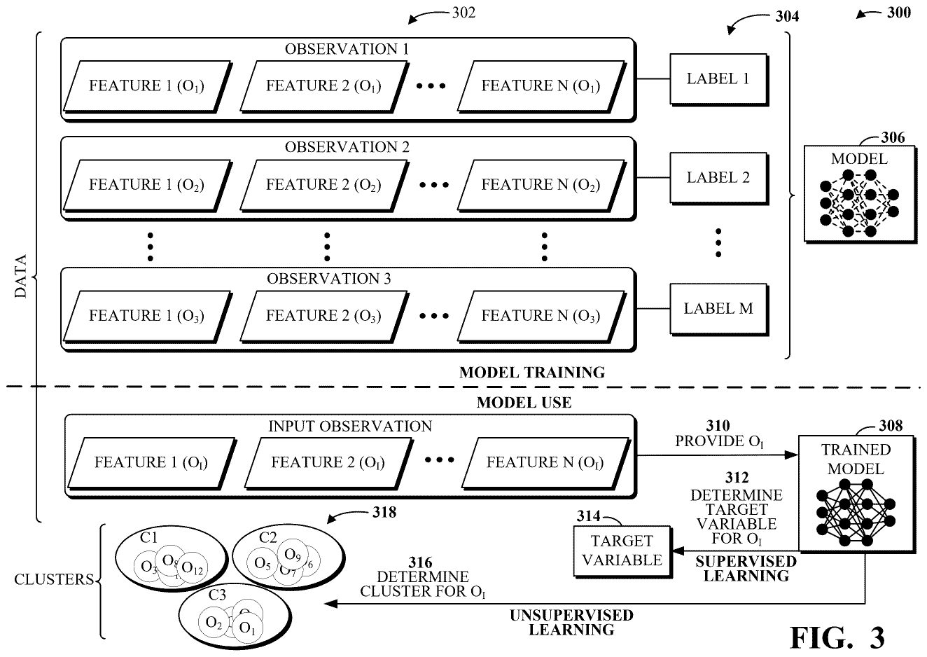

FIG. 3 is a block schematic diagram associated with ML. Specifically, FIG. 3 is a diagram illustrating an example 300 of training and using an ML model. The ML model training and usage described herein may be performed using an AI system (e.g., the AI system 100 shown in FIG. 1A). The AI system may include or may be included in a computing device, a server, a cloud computing environment, and/or the like. The example 300 includes an observation dataset 302, labels 304, a model 306, a trained model 308, an input observation 310, operations 312 and 316, a target variable 314, and clusters 318 including a first cluster C1, a second cluster C2, and a third cluster C3.

The observation dataset 302 comprises a set of observations, each containing multiple features (FEATURE 1 (O1), FEATURE 2 (O2), etc.). This dataset serves as the foundation for training a machine learning model. In some embodiments, the observation dataset 302 may be obtained from historical healthcare claims data, including information such as patient demographics, diagnosis codes, procedure codes, and claim outcomes. Some implementations may incorporate data from electronic health records, practice management systems, or payer portals to enrich the observation dataset.

The features may include a set of variables, and a variable may be referred to as a feature. A specific observation may include a set of variable values (or feature values) corresponding to the set of variables. As an example, a feature set for a set of observations may include a first feature of feature 1 data, a second feature of feature 2 data, a third feature of feature 3 data, and so on. As shown, for a first observation, the first feature may have a value of feature 1 data 1, the second feature may have a value of feature 2 data 1, the third feature may have a value of feature 3 data 1, and so on. These features and feature values are provided as examples and may differ in other examples. In some implementations, the AI system may determine variables for a set of observations and/or variable values for a specific observation based on input received from the AI system. For example, the AI system may identify a feature set (e.g., one or more features and/or feature values) by extracting the feature set from structured data, by performing natural language processing to extract the features from unstructured data, by receiving input from an operator, and/or the like.

Labels 304 are associated with each observation in the dataset 302. These labels represent known outcomes or classifications for the training data. In the context of healthcare revenue cycle management, labels may indicate whether a claim was approved, denied, or partially paid. Some implementations may use labels to represent other outcomes, such as the likelihood of claim denial or the expected time to payment. A label may represent a variable having a numeric value, may represent a variable having a numeric value that falls within a range of values or has some discrete possible values, may represent a variable that is selectable from one of multiple options, may represent a variable having a Boolean value, and/or the like.

The target variable may represent a value that an ML model is being trained to predict, and the feature set may represent the variables that are input to a trained ML model to predict a value for the target variable. The set of observations may include target variable values (e.g., labels) so that the machine learning model can be trained to recognize patterns in the feature set that lead to a target variable value. A machine learning model that is trained to predict a target variable value may be referred to as a supervised learning model.

In some implementations, the machine learning model may be trained on a set of observations that do not include a label. This may be referred to as an unsupervised learning model. In this case, the machine learning model may learn patterns from the set of observations without labeling or supervision, and may provide output that indicates such patterns, such as by using clustering and/or association to identify related groups of items within the set of observations.

The model 306 represents the initial, untrained machine learning algorithm. This model may take various forms, such as a neural network, decision tree, random forest, or support vector machine. In some embodiments, the model 306 may be a combination of multiple algorithms, forming an ensemble model to improve overall performance. Some implementations may employ different model architectures based on the specific requirements of the healthcare revenue cycle management task at hand.

Through the training process, the model 306 is transformed into the trained model 308. This trained model has learned patterns and relationships from the observation dataset 302 and associated labels 304. In the context of the present disclosure, the trained model 308 may be capable of predicting claim outcomes, identifying potential denials, or detecting anomalies in billing patterns. Some embodiments may produce trained models specialized in other aspects of revenue cycle management, such as optimizing coding practices or predicting patient payment behavior.

The input observation 310 represents new, unseen data that is fed into the trained model 308 for analysis or prediction. In healthcare revenue cycle management, an input observation might be a newly submitted claim or a set of claims awaiting processing. The input observation 310 contains the same types of features as the training data but without known labels or outcomes. Some implementations may allow for real-time input of claim data as it is generated, enabling immediate analysis and decision support.

Operation 312 refers to the process of applying the trained model 308 to the input observation 310 to determine a target variable 314 for the input observation 310. This operation may involve various computational steps, depending on the type of model used. For example, in a neural network, operation 312 would involve forward propagation of the input data through the network layers. In a decision tree model, it would involve traversing the tree based on the input features. Some implementations may incorporate additional pre-processing steps or feature engineering techniques as part of operation 312 to optimize model performance.

The target variable 314 is the output produced by the trained model 308 after processing the input observation 310. In the context of healthcare revenue cycle management, the target variable 314 might represent a predicted probability of claim approval, an estimated payment amount, or a classification of the claim into different risk categories. Some implementations may generate multiple target variables simultaneously, providing a more comprehensive analysis of each claim or set of claims.

Operation 316 represents an unsupervised learning process in which a cluster is determined for the input observation 314. The clusters 318, including first cluster C1, second cluster C2, and third cluster C3, illustrate the model's ability to group similar observations together. In the context of healthcare revenue cycle management, these clusters might represent groups of claims with similar characteristics, denial patterns, or payment behaviors. For example, cluster C1 might contain claims likely to be approved without issue, cluster C2 might represent claims at high risk of denial, and cluster C3 could indicate claims requiring additional documentation. Some implementations may employ more sophisticated clustering techniques, such as hierarchical clustering or density-based clustering, to identify more nuanced patterns in the data.

The machine learning system depicted in FIG. 3 can be applied to various aspects of healthcare revenue cycle management beyond claim prediction and classification. For instance, it could be used to optimize billing workflows by identifying the most effective times or methods for submitting claims to different payers. The system could also be employed to detect potential fraud or abuse by identifying unusual patterns in claiming behavior that deviate significantly from established norms.

Another potential application of the machine learning system is in predicting patient payment behavior. By analyzing historical patient data, demographic information, and payment patterns, the system could help healthcare providers anticipate which patients are likely to pay their bills promptly, which might need payment plans, and which may require more aggressive collection efforts. This could lead to more personalized and effective financial counseling and billing strategies.

The machine learning system of the present disclosure is not limited to supervised learning approaches as depicted in FIG. 3. Some implementations may incorporate unsupervised learning techniques to discover hidden patterns in healthcare revenue cycle data without the need for labeled training data. For example, anomaly detection algorithms could be employed to identify unusual claims or billing practices that may warrant further investigation, even if they don't fit into predefined categories of known issues.

In some embodiments, the machine learning system may be extended to include reinforcement learning capabilities. This approach could be particularly useful in optimizing long-term revenue cycle strategies. For instance, a reinforcement learning model could learn to balance the trade-offs between aggressive claim submission (which might lead to more denials but faster payments) and more conservative approaches (which might have higher approval rates but slower cash flow). The model could adapt its strategies over time based on the observed outcomes and financial impact of different approaches.

Similar to human learning, RL trains neural networks through trial and error. Specifically, the neural network produces an output, receives feedback regarding this output, and then learns from the feedback. For instance, when finetuning a language model using reinforcement learning from human feedback (RLHF), the language model generates text and receives a score or reward from a human annotator, which reflects the quality of the text. The AI training software then employs RL to finetune the language model to generate outputs with high scores.

Reinforcement learning proves to be an advantageous and promising learning algorithm for neural networks because it allows learning from non-differentiable signals, which are incompatible with supervised learning. This capability enables the AI training software to learn from arbitrary feedback on a neural network's output. In the case of RLHF, the outputs generated by a language model can be scored according to any predefined principle. The AI training software then uses RL to learn from these scores, regardless of their definition.

Problems addressed via RL are typically structured in a consistent format. Specifically, an agent interacts with an environment, maintaining a state within this environment and producing actions that can alter the current state. As the agent interacts with the environment, it can receive both positive and negative rewards for its actions. The agent's objective is to maximize the rewards received, although not every action is associated with a reward. Rewards may have a long horizon, necessitating several correct, consecutive actions to generate any positive reward. In mathematical terms, RL may be described as a Markov decision process (MDP). An MDP includes states, actions, rewards, transitions, and a policy. States and actions have discrete values, while rewards are real numbers. In an MDP, a policy (referred to herein, interchangeably as a “policy model”) takes a state as input and outputs a probability distribution over possible actions. Given this output, a decision can be made for the action to be taken from a current state, and the transition is then a function that outputs the next state based upon the prior state and chosen action. Using these components, the agent can interact with the environment in an iterative fashion to generate a trained policy.

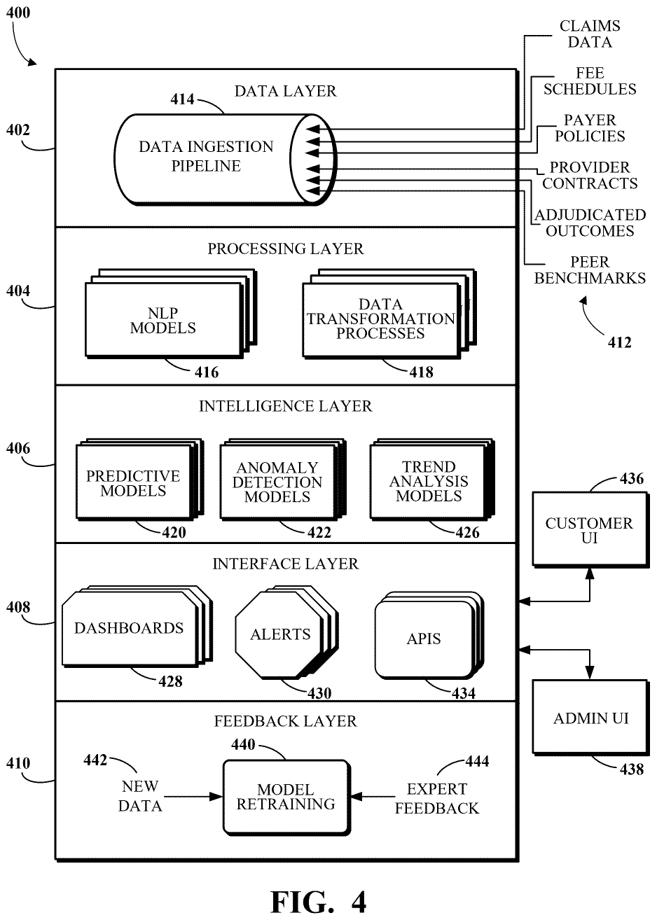

FIG. 4 illustrates a block schematic diagram of an example 400 associated with processing data to provide intelligent informational services according to aspects of the present disclosure. As shown, the example 400 includes an AI system 400. The AI system 400 may be, be similar to, include, or be included in, the AI system 100 shown in FIG. 1A. The AI system 400 includes multiple functional layers including a data layer 402, a processing layer 404, an intelligence layer 406, an interface layer 408, and a feedback layer 410.

The data layer 402 may serve as the foundation of the AI system 400, receiving input data 412 through a data ingestion pipeline 414. The input data 412 may include a wide variety of healthcare revenue cycle information such as claims data, fee schedules, payer policies, provider contracts, adjudicated outcomes, and peer benchmarks. In some implementations, the input data 412 may also encompass patient demographic information, clinical data from EHRs, and historical billing patterns. In some implementations, the system may incorporate real-time data streams from connected medical devices or wearables to provide a more comprehensive view of patient health and associated billing implications.

The data ingestion pipeline 414 may be responsible for collecting, validating, and standardizing the diverse input data 412. This pipeline may employ various data integration techniques, including API connections, SFTP file transfers, and database replication, to ensure a continuous flow of up-to-date information. In some implementations, the data ingestion pipeline 414 may incorporate blockchain technology to enhance data integrity and traceability, particularly for sensitive healthcare information.

The processing layer 404 may include advanced data profiling capabilities to analyze and understand the structure, content, and quality of incoming data from various sources. Data profiling techniques may involve statistical analysis, pattern recognition, and metadata extraction to identify data types, formats, relationships, and anomalies within the input data 412. In some implementations, the system may employ machine learning algorithms to automate the data profiling process, enabling the discovery of complex data patterns and interdependencies that may not be apparent through manual inspection.

In some implementations, the data layer 402 and/or the processing layer 404 may be configured to perform a data profiling information. In some implementations, the data profiling operation may involve using an ML-based data profiling tool to analyze first data associated with a first data source and generate first data profile content based on the first data. The first data profile content may identify first data schema content associated with the first data. In some implementations, the ML-based data profiling tool may be used to analyze second data associated with a second data source and generate second data profile content, which may identify second data schema content associated with the second data. In some aspects, the first and second data profile content may further identify data type information associated with their respective data sources. The data profiling operation may employ various data analysis techniques, such as statistical analysis, pattern recognition, and metadata extraction, to identify data types, formats, relationships, and anomalies within the input data

Schema matching techniques may be performed by the processing layer 404 to align and map data elements from disparate sources to a unified schema. This process may involve identifying semantic and structural similarities between different data schemas, such as matching column names, data types, and value ranges. In some implementations, the system may use NLP algorithms to interpret and match schema elements based on their descriptions or associated metadata. The schema matching process may be iterative, with the system learning and improving its matching accuracy over time based on user feedback and observed data patterns.

Data standardization and cleaning operations may be performed to ensure consistency and accuracy across the unified dataset. These processes may include tasks such as normalizing data formats, resolving inconsistencies in naming conventions, and harmonizing units of measurement. In some implementations, the system may apply domain-specific rules and transformations, such as standardizing medical codes (e.g., converting between ICD-9 and ICD-10) or normalizing provider identifiers across different systems. Machine learning models may be employed to detect and correct data quality issues, such as identifying and imputing missing values or flagging potential data entry errors.

In some implementations, a data integration process that may be performed at least partially within the processing layer 404 may involve combining and reconciling data from multiple sources into a coherent and unified view. This may include resolving conflicts between overlapping data elements, merging duplicate records, and establishing relationships between entities across different datasets. In some implementations, the system may implement probabilistic matching algorithms to link patient records or claims data across disparate systems, even in the absence of perfect identifier matches.

Advanced entity resolution techniques may be applied as part of the data integration process to identify and link related entities across different data sources. This may involve using fuzzy matching algorithms, phonetic encoding, and machine learning-based similarity measures to reconcile variations in entity names, addresses, or other identifying information. In some implementations, the system may maintain a master data management (MDM) component to ensure consistent representation of key entities such as patients, providers, and payers across the integrated dataset.

The processing layer 404 may incorporate data lineage tracking capabilities to maintain transparency and auditability throughout the data integration process. This may involve recording the origin, transformations, and dependencies of each data element as it moves through the system. In some implementations, the data lineage information may be used to support data governance initiatives, facilitate troubleshooting of data issues, and enable the system to automatically update or reprocess affected data elements when source data changes.

Temporal aspects of data integration may be addressed within the processing layer 404 to handle time-sensitive information and historical data effectively. This may include strategies for managing data with different update frequencies, reconciling timestamp discrepancies across systems, and maintaining historical snapshots for trend analysis. In some implementations, the system may employ temporal databases or time series data structures to efficiently store and query time-varying data, such as changes in patient insurance coverage or evolving payer policies.

The data integration capabilities of the processing layer 404 may extend to handling semi-structured and unstructured data sources, such as clinical notes, medical images, or PDF documents. In these cases, the system may employ techniques such as optical character recognition (OCR), NLP, or computer vision algorithms to extract relevant information and integrate it with structured data sources. This comprehensive approach to data integration may enable the AI system 400 to leverage a wide range of information sources, providing a more holistic view of the healthcare revenue cycle and supporting more sophisticated analytics and decision-making processes.

The processing layer 404 contains NLP models 416 and data transformation processes 418 that process the ingested data. The NLP models 416 may be designed to extract meaningful information from unstructured text data, such as clinical notes, denial reasons, or payer correspondence. These models may utilize techniques like named entity recognition, sentiment analysis, and topic modeling to convert free-text information into structured, analyzable data. In some implementations, the NLP models 416 may be fine-tuned on domain-specific healthcare vocabularies to improve accuracy in medical terminology extraction.

Data transformation processes 418 in the processing layer 404 may be responsible for cleaning, normalizing, and enriching the data. These processes may include tasks such as standardizing medical codes (e.g., converting between ICD-9 and ICD-10), resolving data inconsistencies, and imputing missing values. In some implementations, the system may incorporate advanced data quality algorithms that use machine learning to detect and correct anomalies in the data automatically. For example, the system may employ a combination of rule-based and statistical approaches to identify and rectify coding errors, such as mismatched procedure and diagnosis codes or invalid modifier combinations. In some implementations, the data transformation processes may utilize natural language processing techniques to extract structured information from unstructured clinical notes, enhancing the richness of the available data for analysis.

The data transformation process within the processing layer 404 may involve a multi-stage approach to convert raw input data into a standardized, analyzable format. In some implementations, the system may employ a combination of rule-based transformations and machine learning algorithms to handle the diverse range of data types and formats encountered in healthcare revenue cycle management. The transformation process may begin with data parsing, where incoming data is broken down into its constituent elements based on predefined schemas or learned patterns. This parsing step may be particularly important for handling complex data structures such as ANSI X12 EDI files, which contain multiple nested segments and loops.

Following the parsing stage, the system may apply a series of data cleansing operations to address quality issues and inconsistencies. These operations may include removing duplicate records, standardizing date formats, correcting spelling errors in free-text fields, and resolving conflicting information across different data sources. In some implementations, the system may utilize fuzzy matching algorithms to identify and merge records that likely refer to the same entity but contain slight variations in identifying information. For example, the system may determine that “John Doe” and “Jon Doe” with matching dates of birth and addresses are likely the same patient, and consolidate their records accordingly.

The data transformation process may also involve semantic enrichment, where additional context and meaning are added to the raw data. This may include mapping local codes to standardized terminologies (e.g., converting proprietary service codes to CPT codes), inferring missing information based on available data (e.g., deriving a patient's age from their date of birth), and linking related data elements across different sources (e.g., associating a claim with relevant clinical documentation). In some implementations, the system may leverage external knowledge bases or ontologies to enhance the semantic richness of the transformed data, enabling more sophisticated analytics and decision support capabilities.

The intelligence layer 406 forms the analytical core of the AI system 400, comprising predictive models 420, anomaly detection models 422, and trend analysis models 426. Predictive models 420 may be designed to forecast various aspects of the revenue cycle, such as the likelihood of claim denials, expected reimbursement amounts, or patient payment behavior. These models may employ a range of machine learning techniques, including logistic regression, random forests, or gradient boosting machines, depending on the specific prediction task.

The predictive models 420 may incorporate a variety of machine learning algorithms and techniques to forecast different aspects of the healthcare revenue cycle. In some implementations, the system may utilize ensemble methods, combining multiple models to improve overall prediction accuracy and robustness. For example, a random forest classifier may be used in conjunction with a gradient boosting machine to predict the likelihood of claim denials, leveraging the strengths of both algorithms to capture complex patterns in the data.

The predictive models 420 may be trained on historical claims data, including features such as patient demographics, diagnosis codes, procedure codes, provider information, and payer details. In some implementations, the system may employ transfer learning techniques to adapt pre-trained models to specific healthcare organizations or specialties, allowing for faster model deployment and improved performance on smaller datasets. The models may also incorporate temporal features, such as seasonality and trends in claim adjudication patterns, to capture time-dependent variations in the revenue cycle.

In some implementations, the predictive models 420 may utilize deep learning architectures, such as recurrent neural networks (RNNs) or transformer models, to analyze sequential data in the claims processing workflow. These models may be particularly effective in capturing long-term dependencies and complex interactions between different stages of the revenue cycle. For example, an RNN-based model may be used to predict the expected time to payment for a claim, taking into account the entire history of interactions between the healthcare provider and the payer.

The system may also employ interpretable machine learning techniques to provide insights into the factors driving the predictions. In some implementations, the predictive models 420 may generate feature importance scores or use techniques like SHAP (SHapley Additive exPlanations) values to explain which input variables have the most significant impact on the predicted outcomes. This interpretability may enable healthcare providers to understand the underlying reasons for predicted claim denials or reimbursement amounts, facilitating targeted interventions and process improvements in their revenue cycle management.

Anomaly detection models 422 in the intelligence layer 406 may be useful for identifying unusual patterns or outliers in the revenue cycle data. These models may use techniques such as isolation forests, autoencoders, or clustering algorithms to flag potential issues like fraudulent claims, coding errors, or sudden changes in payer behavior. In some implementations, the anomaly detection models 422 may incorporate time series analysis to detect temporal anomalies, such as unexpected spikes in denial rates or changes in payment patterns over time.

The anomaly detection models 422 in the intelligence layer 406 may employ a combination of supervised and unsupervised machine learning techniques to identify unusual patterns or outliers in the healthcare revenue cycle data. These models may analyze various features of claims data, including but not limited to claim amounts, diagnosis codes, procedure codes, provider information, and payer behavior. In some implementations, the system may utilize isolation forests, which work by isolating anomalies in the feature space based on the principle that anomalies are rare and different. This approach may be particularly effective in detecting fraudulent claims or unusual billing patterns that deviate significantly from the norm.

In addition to isolation forests, the anomaly detection models 422 may incorporate autoencoder neural networks to identify anomalies in high-dimensional data. Autoencoders may be trained on normal revenue cycle data to learn a compressed representation of the input features. When presented with new data, the autoencoder may attempt to reconstruct the input from its compressed representation. Data points that result in high reconstruction errors may be flagged as potential anomalies. This technique may be useful for detecting subtle deviations in claim characteristics or payer behavior that might not be apparent through traditional rule-based approaches. Furthermore, the system may employ time series analysis techniques, such as ARIMA models or Prophet, to detect temporal anomalies in the revenue cycle data. These models may help identify unexpected spikes in denial rates, sudden changes in payment patterns, or shifts in payer adjudication practices over time.

Trend analysis models 426 may be responsible for uncovering long-term patterns and relationships in the revenue cycle data. These models may employ techniques like time series decomposition, seasonal trend analysis, or causal inference methods to identify underlying factors affecting financial performance. In some implementations, the system may incorporate advanced forecasting techniques like Prophet or ARIMA models to provide more accurate long-term revenue projections.

The interface layer 408 may serve as the bridge between the AI system's analytical capabilities and its users, providing multiple output interfaces including dashboards 428, alerts 430, and APIs 434. Dashboards 428 may offer visual representations of key performance indicators, trends, and insights derived from the intelligence layer. These dashboards may be interactive, allowing users to drill down into specific data points or customize views based on their roles and preferences. In some implementations, the dashboards 428 may incorporate augmented reality (AR) features for immersive data exploration experiences.

Alerts 430 in the interface layer 408 may provide timely notifications of important events, anomalies, or actionable insights detected by the AI system. These alerts may be delivered through various channels such as email, SMS, or in-app notifications, and can be customized based on user roles and preferences. In some implementations, the system may include a smart alerting system that uses machine learning to prioritize and contextualize alerts, reducing alert fatigue and ensuring that users receive the most relevant information.

The AI system may organize metrics into a hierarchical structure to facilitate efficient analysis and alert generation. At the core of this structure are individual metrics, which represent specific measurable aspects of the healthcare revenue cycle. These metrics may include various types such as denial rates, claims submission volumes, accounts receivable aging, or reimbursement rates. Each metric may be associated with a particular time frame and aggregation method, forming what may be referred to as a metric subtype. For example, a “14-day rolling average denial rate” or a “60-day standard deviation of rolling rate” may constitute different subtypes of the denial rate metric.

Metrics may be grouped into metric groups, which represent collections of related metrics that provide a comprehensive view of a particular aspect of the revenue cycle. For instance, a denial-related metric group may include metrics such as overall denial rate, denial rate by reason code, and average time to resolve denials. The system may allow for flexible definition of metric groups, enabling healthcare organizations to tailor their analytics to specific operational needs or strategic priorities. Furthermore, the AI system may implement a segmentation approach to provide granular insights into metric performance across different dimensions of the healthcare business. Segments may include categories such as payer, facility, medical group, or rendering provider group. By applying these segments to metrics and metric groups, the system may generate multidimensional analyses that allow users to drill down into specific areas of concern or identify patterns across different operational units.

The AI system may structure data to support alerts through a comprehensive and flexible data model. This model may include several key components designed to capture, organize, and facilitate the generation and management of alerts within the healthcare revenue cycle management context. At the core of the alert data structure may be an Alert table. This table may contain essential information about each alert, including a unique identifier, alert type, alert date, alert name, status, and description. The alert type field may categorize alerts into different classes, such as denials, accounts receivable, or revenue-related issues. The alert date may represent the business-relevant date for the alert, which could differ from the system's creation timestamp.

The Alert table may be associated with a MetricGroup table through a foreign key relationship. Each alert may be linked to a specific metric group, which represents a set of related metrics used to generate the alert. The MetricGroup table may store information about the type of metrics it contains, such as AR (accounts receivable) or denials, and may include a JSON structure that defines the set of segments used to filter and group the metrics. To support flexible segmentation of metrics and alerts, the system may implement a MetricSegment table. This table may define the specific dimensions or filters applied to a metric group, such as payer, facility, medical group, or rendering provider group. Each metric segment may have an order field, allowing for prioritization of segments within a metric group. This structure may enable the system to generate alerts based on multi-dimensional analysis of the revenue cycle data.

The system may utilize separate metric tables for different types of financial data. For example, there may be Metric_AR_Bin and Metric_AR_Agg tables for storing accounts receivable metrics at different levels of granularity. These tables may contain fields for specific AR-related measurements, such as AR amounts for different time bins (0-30 days, 31-60 days, etc.) or aggregated AR totals. Similar tables may exist for other metric types, such as denials or remittances.

To maintain a historical record of metrics and support trend analysis for alerts, the system may implement a MetricLog table. This table may store all versions of metrics over time, allowing the system to track changes and generate alerts based on historical comparisons. A corresponding MetricCurrent table may maintain the latest version of each metric, enabling efficient querying for the most up-to-date information when generating alerts. The alert data structure may include an AlertMetric table to establish the relationship between alerts and the specific metrics that triggered them. This table may serve as a bridge, linking each alert to one or more metrics from the MetricLog or MetricCurrent tables. This structure may allow the system to provide detailed context and supporting data for each generated alert.

To support user interaction and feedback on alerts, the system may implement an AlertUser table. This table may store information about user interactions with alerts, such as whether an alert has been viewed, acknowledged, or acted upon. It may also capture user feedback, such as the perceived relevance or usefulness of the alert, which can be used to improve future alert generation and prioritization. The data model may include an AlertGroup table to support the grouping of related alerts. This feature may be particularly useful for tracking the progression of issues over time or for consolidating multiple alerts related to a single root cause. The AlertGroup table may have a many-to-many relationship with the Alert table, allowing for flexible grouping of alerts based on various criteria.

To enhance the context and actionability of alerts, the system may implement an AlertAction table. This table may store recommended actions or next steps associated with each alert type. By linking alerts to specific actions, the system may provide users with guidance on how to address the issues identified by the alerts, improving the overall efficiency of the revenue cycle management process. The alert data structure may also include support for alert prioritization through an AlertPriority table. This table may define different priority levels and their associated characteristics, such as response time requirements or escalation procedures. By assigning priorities to alerts, the system may help users focus on the most critical issues affecting the revenue cycle.

To facilitate the customization of alert delivery and presentation, the system may implement an AlertProfile table. This table may store user-specific or role-specific preferences for alert notifications, such as delivery channels, frequency, and visualization options. By tailoring the alert experience to individual users or roles, the system may improve the relevance and effectiveness of the alerting mechanism.

In some implementations, the AI system may utilize a single, comprehensive Metric table to capture all the information described in the previous data model. This unified Metric table may incorporate fields to represent various types of metrics, including accounts receivable, denials, remittances, and other financial indicators. The table may include columns for metric type, metric subtype, metric value, metric date, and aggregation method, allowing for flexible storage of different metric categories and subtypes within a single structure.

To support the hierarchical organization and segmentation of metrics, the Metric table may include additional columns for metric group, segment type, and segment value. These fields may allow for the representation of metric groups and multi-dimensional segmentation without the need for separate MetricGroup and MetricSegment tables. The table may also incorporate a version column and a timestamp to maintain historical records and support trend analysis, effectively combining the functionality of the previously described MetricLog and MetricCurrent tables. This approach may simplify data retrieval and reduce the need for complex joins when generating alerts or populating dashboards, potentially improving query performance and system scalability.

APIs 434 may enable seamless integration of the AI system's capabilities with other healthcare IT systems, such as electronic health records (EHRs), practice management software, or third-party analytics tools. These APIs may support both data ingestion and output, allowing for bidirectional information flow. In some implementations, the APIs 434 may incorporate GraphQL for more flexible and efficient data querying, or implement OAuth 2.0 for enhanced security in data exchange.

The interface layer 408 connects to both a customer UI 436 and an administrator UI 438, catering to different user roles within the healthcare organization. The customer UI 436 may be designed for end-users such as billing specialists, revenue cycle managers, or financial analysts, providing intuitive access to insights and functionalities relevant to their daily operations. The administrator UI 438, on the other hand, may offer more advanced configuration options, system monitoring tools, and access to detailed performance metrics.

The AI system 400 may continually learn and improve through the feedback layer 410. This layer contains a model retraining component 440 configured to train and/or retrain one or more ML models. In some implementations, the model retraining component 440 may implement one or more aspects of ML training described above in connection with FIG. 3. The model retraining component 440 may receive new data 442 and expert feedback 444. The new data 442 may include the latest claims data, updated payer policies, or recent adjudication outcomes, allowing the system to adapt to changing patterns in the healthcare landscape. Expert feedback 444 may come from healthcare professionals, billing specialists, or domain experts who can provide valuable insights or corrections to the system's outputs.

The model retraining component 440 may use this input to update and refine the various models in the intelligence layer 406. This may involve techniques such as online learning, transfer learning, or periodic batch retraining to incorporate new information without compromising the stability of existing models. In some implementations, the model retraining component 440 may employ automated machine learning (AutoML) techniques to continuously optimize model architectures and hyperparameters based on performance metrics and new data.

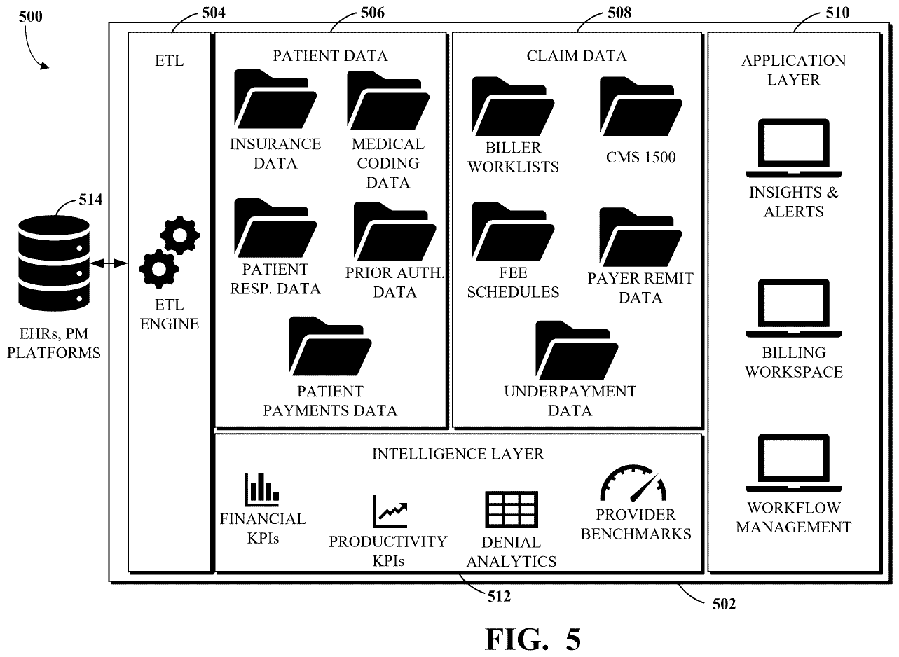

FIG. 5 illustrates a block diagram of an example of an AI system 500 for providing intelligent informational services associated with healthcare workflows according to aspects of the present disclosure. The AI system 500 comprises multiple interconnected components designed to process, analyze, and optimize healthcare revenue cycle management data. The AI system 500 may be, be similar to, include, or be included in, the AI system 100 shown in FIG. 1A.

The AI system 500 includes an application interface 502 that serves as the primary point of interaction for users of the system. This interface may be implemented as a web-based portal, a mobile application, or a desktop client, providing access to various features and functionalities of the AI system 500. For example, the application interface 502 may offer customizable dashboards for different user roles, such as billing specialists, revenue cycle managers, or financial analysts. In some implementations, the application interface 502 may incorporate voice-activated controls or augmented reality displays for hands-free operation in clinical settings.

A data processing engine 504 forms a component of the AI system 500, responsible for ingesting, cleaning, and transforming raw data from various sources. This engine may employ advanced ETL (Extract, Transform, Load) processes, utilizing machine learning algorithms to automate data cleansing and normalization tasks. For instance, the data processing engine 504 may use natural language processing techniques to extract relevant information from unstructured clinical notes or payer correspondence. In some implementations, the data processing engine 504 may incorporate blockchain technology to ensure data integrity and traceability throughout the revenue cycle management process.

The AI system 500 includes a patient data repository 506 that stores comprehensive information related to patient care and billing. This repository may contain various types of data, including medical coding information, insurance details, prior authorization records, patient response data, and patient payment history. For example, the medical coding data might include ICD-10 diagnosis codes, CPT procedure codes, and HCPCS codes for medical supplies and equipment. In some implementations, the patient data repository 506 may utilize advanced data compression techniques or implement a data lake architecture to efficiently store and manage large volumes of diverse patient information.

A claim data repository 508 within the AI system 500 is dedicated to storing and managing claim-related information. This repository may include data types such as CMS 1500 form data, biller worklists, fee schedules, payer remit data, and underpayment information. For instance, the CMS 1500 data might encompass details like patient demographics, insurance information, diagnoses, and procedures performed. In some implementations, the claim data repository 508 may employ a graph database structure to efficiently represent and query complex relationships between claims, patients, providers, and payers.

The AI system 500 features an application layer 510 that houses key functional components for revenue cycle management. This layer may include modules for insights and alerts, billing workspace, and workflow management. For example, the insights and alerts component may use machine learning algorithms to detect unusual patterns in claim denials or identify potential coding errors before submission. The billing workspace might offer an intuitive interface for managing claims throughout their lifecycle, while the workflow management component may optimize task allocation among billing staff. In some implementations, the application layer 510 may incorporate robotic process automation (RPA) capabilities to automate repetitive tasks within the revenue cycle workflow.

An intelligence layer 512 within the AI system 500 provides advanced analytical capabilities to support decision-making and process optimization. This layer may offer functionalities such as financial KPIs, productivity KPIs, denial analytics, and provider benchmarks. For instance, the financial KPIs might include metrics like days in accounts receivable, clean claim rate, or collection rate, while productivity KPIs may track metrics such as claims processed per hour or denial resolution time. In some implementations, the intelligence layer 512 may utilize reinforcement learning techniques to continuously optimize revenue cycle strategies based on observed outcomes and financial impact.

The AI system 500 interacts with various data sources 514, which may include EHR systems, practice management software, clearinghouse services, payer portals, and financial management systems. These data sources provide the raw input that fuels the AI system's analytical capabilities. For example, an EHR system might supply clinical documentation and charge capture data, while a clearinghouse service may provide claim submission and response information. In some implementations, the AI system 500 may incorporate edge computing nodes to process and analyze data from internet-of-things (IoT) medical devices or wearables, providing real-time insights into patient health and potential billing implications.

The components of the AI system 500 are interconnected through an application interface 502, which facilitates seamless data flow and communication between different modules. This interface may utilize standardized healthcare data exchange protocols, such as HL7 FHIR or X12 EDI, to ensure interoperability with external systems. In some implementations, the application interface 502 may employ a microservices architecture, allowing for greater flexibility and scalability in deploying and updating individual components of the AI system 500. Some aspects might incorporate an event-driven architecture to enable real-time responsiveness to changes in the healthcare revenue cycle landscape.

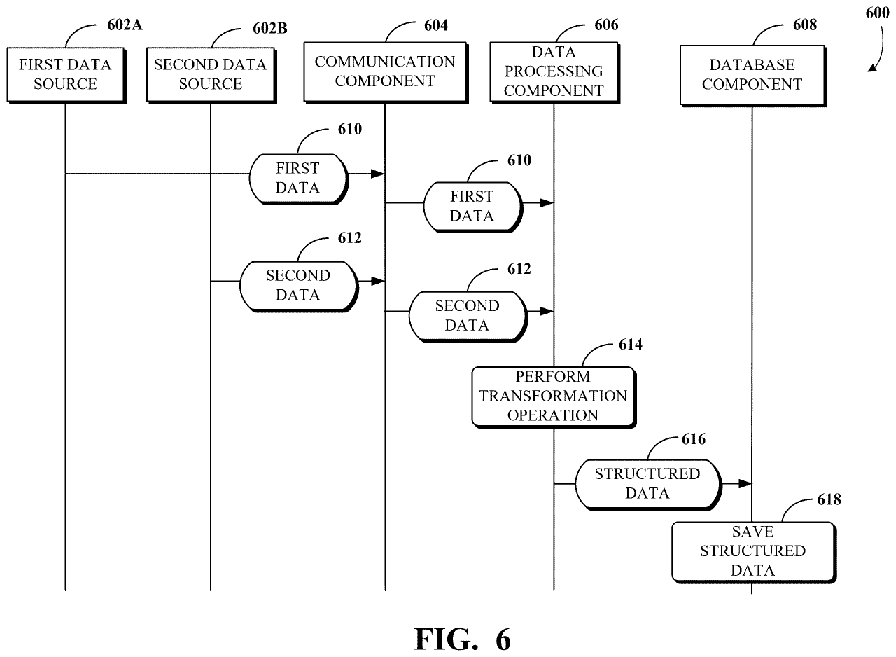

FIG. 6 illustrates a flow diagram of an example of a process 600 for processing data from disparate data sources according to aspects of the present disclosure. The process 600 depicts the flow of data through various components of an AI system for healthcare revenue cycle management.

The process 600 begins with data ingestion from a first data source 602A and a second data source 602B. These data sources may represent different systems commonly used in healthcare organizations, such as EHR systems, practice management software, clearinghouse services, payer portals, or financial management systems. For example, the first data source 602A could be an Epic EHR system containing clinical documentation and charge capture data, while the second data source 602B might be a Waystar clearinghouse service providing claim submission and response information. In some implementations, the process 600 may incorporate additional data sources, such as real-time patient monitoring devices or wearable health trackers, to provide a more comprehensive view of patient health and potential billing implications.

A communication component 604 receives and manages the data flow from these sources. This component may employ various data integration techniques to ensure efficient and secure data transfer. For instance, the communication component 604 might utilize HL7 FHIR (Fast Healthcare Interoperability Resources) standards for exchanging healthcare information electronically, or implement X12 EDI (Electronic Data Interchange) protocols for standardized business communication. In some implementations, the communication component 604 may incorporate blockchain technology to enhance data integrity and traceability throughout the revenue cycle management process.

The data processing component 606 is responsible for processing first data 610 from the first data source 602A and second data 612 from the second data source 602B. This component may employ advanced ETL (Extract, Transform, Load) processes, utilizing machine learning algorithms to automate data cleansing and normalization tasks. For example, the data processing component 606 might use natural language processing techniques to extract relevant information from unstructured clinical notes or payer correspondence. In some implementations, the data processing component 606 could incorporate edge computing capabilities to process and analyze data from IoT medical devices in real-time, providing immediate insights into patient health and potential billing implications.

A function of the data processing component 606 is to perform a transformation operation 614 on the received data. This operation may involve various data integration and normalization techniques to convert disparate data formats into a unified structure. For instance, the transformation operation 614 might standardize medical codes (e.g., converting between ICD-9 and ICD-10), resolve inconsistencies in naming conventions, or harmonize units of measurement across different systems. In some implementations, the transformation operation 614 could employ advanced entity resolution techniques, using probabilistic matching algorithms to link patient records or claims data across disparate systems, even in the absence of perfect identifier matches.

The transformation operation 614 generates structured data 616, which represents the harmonized and normalized version of the input data. This structured data may include various types of information relevant to healthcare revenue cycle management, such as patient demographics, insurance details, diagnosis and procedure codes, claim statuses, and payment information. In some implementations, the structured data 616 could also incorporate derived features or calculated metrics that provide additional insights into revenue cycle performance, such as predicted likelihood of claim denials or estimated time to payment.

A database component 608 is responsible for storing the processed information. The structured data 616 is stored as structured data storage 618 within the database component 608. This storage may utilize advanced database technologies to efficiently manage large volumes of healthcare data. For example, the database component 608 might employ a combination of relational and NoSQL databases to accommodate the diverse types of data encountered in revenue cycle management. In some implementations, the database component 608 could incorporate a data lake architecture to store and analyze large volumes of unstructured and semi-structured data, providing greater flexibility for future analytics and machine learning applications.

The process 600 demonstrates an approach to data integration and transformation in healthcare revenue cycle management. By leveraging AI and machine learning techniques, the system can efficiently process and normalize data from multiple disparate sources, creating a unified data model that supports advanced analytics and decision-making. This approach may address the technical challenges of fragmented data in healthcare IT systems, enabling healthcare providers to gain a holistic view of their revenue cycle and identify opportunities for optimization.

In some implementations, the process 600 could be extended to incorporate real-time data streaming capabilities, allowing for immediate processing and analysis of incoming data. This could enable the system to provide near-instantaneous alerts and insights, such as flagging potential claim denials before submission or identifying sudden changes in payer behavior that might impact revenue. In some implementations, the process could be enhanced with advanced data governance features, such as automated data quality checks, data lineage tracking, and compliance monitoring to ensure adherence to healthcare data privacy regulations like HIPAA.

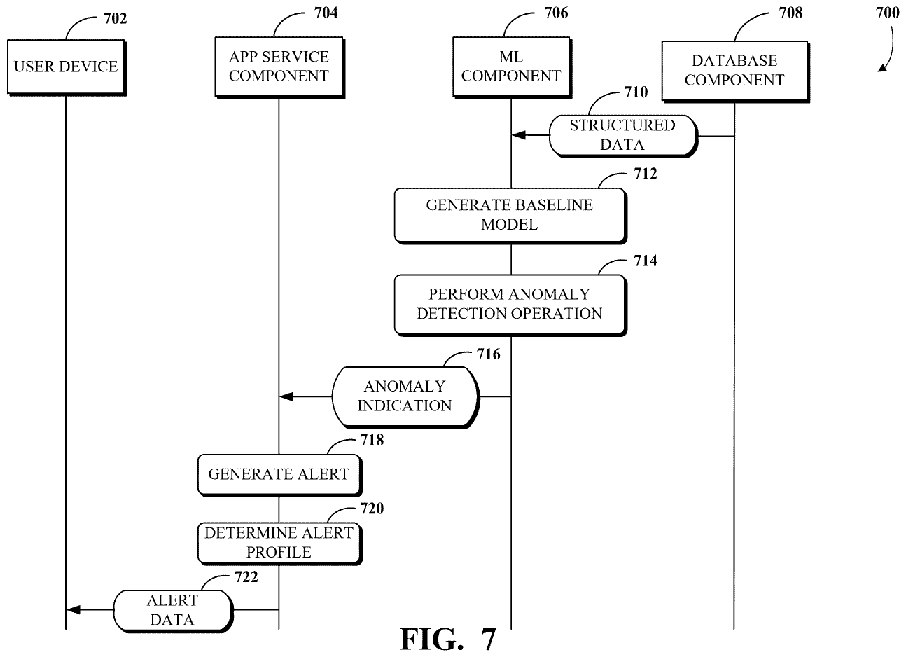

FIG. 7 illustrates a flow diagram of an example of a process 700 for providing alerts associated with healthcare workflows according to aspects of the present disclosure. The process 700 depicts the flow of data through various components of an AI system for healthcare revenue cycle management.

The process 700 begins with a user device 702, which may represent various endpoints through which users interact with the AI system. These devices may include desktop computers, laptops, tablets, smartphones, or specialized healthcare workstations. In some implementations, the user device 702 may be a wearable device or a voice-activated assistant, allowing for hands-free interaction in clinical settings. The user device 702 serves as both an input source for user interactions and the ultimate destination for alert data generated by the system.

An app service component 704 acts as an intermediary between the user device 702 and the core analytical components of the AI system. This component may manage user authentication, handle API requests, and coordinate the flow of information between different parts of the system. In some embodiments, the app service component 704 may implement a microservices architecture, allowing for greater flexibility and scalability in deploying and updating individual components of the AI system. Some implementations might incorporate an event-driven architecture to enable real-time responsiveness to changes in the healthcare revenue cycle landscape.

The ML component 706 represents the machine learning engine of the AI system. This component is responsible for analyzing data and generating insights using various machine learning models. In the context of healthcare revenue cycle management, the ML component 706 may employ a range of techniques, including supervised learning for predicting claim outcomes, unsupervised learning for anomaly detection, and reinforcement learning for optimizing billing workflows. In some implementations, the ML component 706 may utilize ensemble methods, combining multiple models to improve overall prediction accuracy and robustness.

A database component 708 serves as the data storage and retrieval system for the AI platform. This component may utilize advanced database technologies to efficiently manage large volumes of healthcare data. For example, the database component 708 might employ a combination of relational and NoSQL databases to accommodate the diverse types of data encountered in revenue cycle management. In some implementations, the database component 708 could incorporate a data lake architecture to store and analyze large volumes of unstructured and semi-structured data, providing greater flexibility for future analytics and machine learning applications.

The process 700 involves several key operations performed by the ML component 706. The first operation is to generate baseline model 712, which establishes a reference point for normal behavior in the healthcare revenue cycle data. This baseline model may be created using historical claims data, including features such as patient demographics, diagnosis codes, procedure codes, and claim outcomes. In some implementations, the system may employ transfer learning techniques to adapt pre-trained models to specific healthcare organizations or specialties, allowing for faster model deployment and improved performance on smaller datasets.

Following the baseline model generation, the ML component 706 performs an anomaly detection operation 714. This operation analyzes incoming data against the baseline model to identify unusual patterns or deviations that may indicate issues in the revenue cycle. The anomaly detection operation 714 may utilize various techniques such as isolation forests, autoencoders, or clustering algorithms to flag potential problems like fraudulent claims, coding errors, or sudden changes in payer behavior. In some implementations, the anomaly detection operation 714 may incorporate time series analysis to detect temporal anomalies, such as unexpected spikes in denial rates or changes in payment patterns over time.

When the anomaly detection operation 714 identifies a significant deviation from the baseline, it produces an anomaly indication 716. This indication serves as a trigger for the alert generation process. The anomaly indication 716 may include details about the nature of the anomaly, its severity, and the specific data points or trends that contributed to its detection. In some implementations, the system may employ explainable AI techniques to provide insights into why a particular anomaly was flagged, enhancing transparency and trust in the AI-driven alerting process.

Based on the anomaly indication 716, the process generates an alert 718. This step involves transforming the technical anomaly detection results into actionable information for end-users. The alert generation process may consider factors such as the type of anomaly, its potential financial impact, and historical patterns to determine the urgency and relevance of the alert. In some embodiments, the system may use natural language generation techniques to create human-readable alert descriptions that clearly communicate the issue and its implications.

The process then determines an alert profile 720, which tailors the alert presentation based on user-specific or role-specific preferences. This profiling step ensures that alerts are delivered in a manner most relevant and actionable for each user. For example, a billing specialist might receive detailed alerts about specific claims, while a financial executive might see higher-level alerts about overall revenue trends. In some implementations, the alert profile determination may incorporate machine learning to adapt to user behavior over time, improving the relevance and effectiveness of alerts based on how users interact with and respond to them.

Finally, the process generates alert data 722, which is the formatted information sent back to the user device 702 through the app service component 704. This alert data may include various elements such as the alert description, relevant metrics, suggested actions, and links to more detailed information. In some implementations, the alert data may be designed to support interactive visualizations or augmented reality displays, allowing users to explore the underlying data and context of the alert more intuitively.

The process 700 demonstrates a comprehensive approach to AI-driven alerting in healthcare revenue cycle management. By leveraging machine learning for baseline modeling, anomaly detection, and personalized alert generation, the system can provide timely and actionable insights to healthcare providers. This approach may address the technical challenges of identifying and responding to revenue cycle issues in a complex and dynamic healthcare environment, enabling providers to optimize their financial operations and reduce revenue leakage.

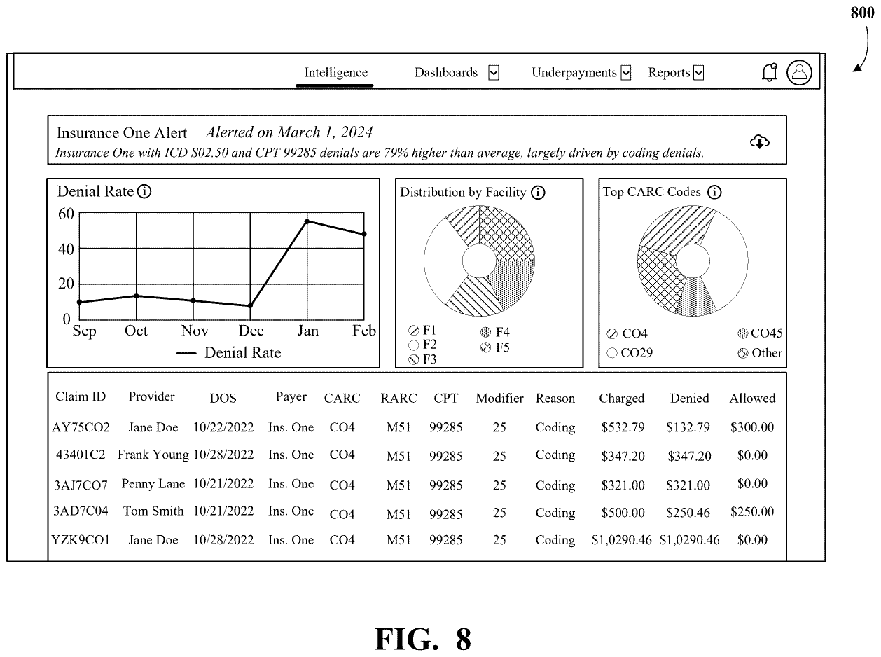

FIG. 8 illustrates a graphical user interface (GUI) 800 for displaying healthcare insurance claim analytics according to aspects of the present disclosure. The GUI 800 presents multiple data visualization components arranged in a dashboard layout, providing users with a comprehensive view of revenue cycle management metrics and insights.

The interface includes a line graph showing denial rate trends over time, with months from September through February plotted on the x-axis and denial rate percentages from 0 to 60 on the y-axis. This visualization may allow users to quickly identify patterns or anomalies in claim denials over the specified time period. In some implementations, the line graph may be interactive, allowing users to hover over data points for more detailed information or select specific time ranges for further analysis.

Adjacent to the line graph are two pie charts—one showing distribution by facility and another displaying top CARC (Claim Adjustment Reason Code) codes. These charts may provide users with a quick overview of how denials are distributed across different healthcare facilities and the most common reasons for claim adjustments. In some implementations, the GUI may offer options to switch between different chart types, such as bar charts or treemaps, to visualize this data in various formats based on user preferences.

The GUI 800 also contains a detailed data table with columns for claim ID, provider name, date of service (DOS), payer, CARC code, RARC (Remittance Advice Remark Code) code, CPT (Current Procedural Terminology) code, modifier, reason, and financial amounts including charged, denied, and allowed values. This table displays multiple claim records with associated information and monetary values, allowing users to drill down into specific claim details. In some implementations, the table may include sorting and filtering capabilities, enabling users to quickly locate and analyze specific subsets of claims based on various criteria.

The interface incorporates navigation elements and interactive components that allow users to access different views of the insurance claim data. These elements may include dropdown menus, tabs, or buttons that enable users to switch between different analytics dashboards or apply various filters to the displayed data. In some implementations, the GUI may incorporate a search functionality that allows users to quickly locate specific claims or providers within the dataset.

The layout of GUI 800 organizes the information in a structured format that enables users to analyze denial rates, claim distributions, and detailed claim information within a single view. This consolidated presentation of data may help healthcare providers quickly identify trends, anomalies, or areas requiring attention in their revenue cycle management processes. In some implementations, the GUI may include customization options that allow users to rearrange or resize different components of the dashboard based on their specific needs or preferences.

The GUI 800 may also incorporate AI-driven insights and recommendations based on the analyzed data. For example, the interface may highlight specific claims or trends that the AI system has identified as requiring immediate attention, or provide suggestions for optimizing billing practices based on historical patterns. In some implementations, the GUI may include a natural language interface that allows users to ask questions about their revenue cycle data and receive AI-generated responses and visualizations.

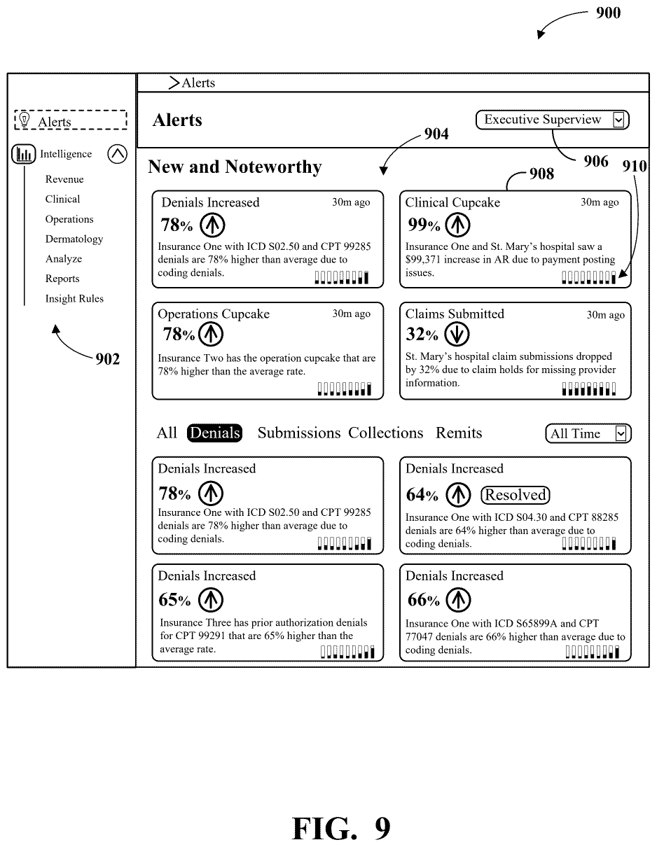

FIG. 9 illustrates a graphical user interface (GUI) 900 for displaying alerts and analytics according to aspects of the present disclosure. The GUI 900 comprises multiple interconnected components designed to provide users with a comprehensive view of revenue cycle management metrics, insights, and alerts.

The GUI 900 includes a navigation menu 902 positioned on the left side of the interface. The navigation menu 902 contains options for accessing various sections of the application, including Alerts, Intelligence, Revenue, Clinical, Operations, Dermatology, Analyze, Reports, and Insight Rules. In some implementations, the navigation menu 902 may be collapsible to provide more screen space for data visualization. Some embodiments may include additional menu items or allow for customization of the menu based on user preferences or role-specific access rights.

The main content area of the GUI 900 features an alert section 904 titled “Alerts” with a “New and Noteworthy” subsection. This alert section 904 may serve as a central hub for displaying important information and anomalies detected by the AI system. The alerts presented in this section may be generated based on various factors, such as unusual claim denial patterns, unexpected changes in revenue, or potential compliance issues. In some implementations, the alert section 904 may include additional categorization options or allow users to customize the types of alerts displayed based on their specific areas of responsibility.

A view selector 906 is positioned in the upper right corner of the GUI 900, allowing users to switch between different views of the data. The current selection shown is “Executive Superview,” which may provide a high-level overview of key performance indicators and important alerts. Other view options might include detailed operational views, financial summaries, or specialty-specific dashboards. In some embodiments, the view selector 906 may incorporate machine learning algorithms to suggest the most relevant view based on the user's role, historical usage patterns, or current system status.

The alert section 904 displays multiple alert cards 908, each presenting various metrics and notifications related to healthcare revenue cycle management. These alert cards 908 may be designed to provide quick, at-a-glance information about specific issues or trends. Each alert card 908 typically includes a percentage value, descriptive text, and a sparkline 910 showing historical trends. The information presented on these cards may include metrics such as denial rates, operations metrics, and claims submission data. In some implementations, the alert cards 908 may be interactive, allowing users to drill down into more detailed information or take immediate action directly from the card interface.

The sparkline 910 featured on each alert card 908 provides a compact, line graph-like visualization of the metric's trend over time. This visual representation may allow users to quickly assess whether a particular metric is improving, declining, or remaining stable. In some embodiments, the sparkline 910 may be color-coded to indicate positive or negative trends, or include markers for significant events or threshold crossings. Some implementations might offer the option to expand the sparkline 910 into a full-sized chart for more detailed analysis.

The alert cards 908 contain detailed information about insurance claims, including specific ICD and CPT codes, percentage changes, and the timing of alerts (e.g., “30m ago”). This level of detail may allow users to quickly understand the nature and context of each alert. In some implementations, the system may use natural language generation techniques to create human-readable alert descriptions that clearly communicate the issue and its implications. Some embodiments might include the ability to customize the information displayed on the alert cards 908 based on user preferences or role-specific requirements.

The interface includes filtering options at the bottom of the alert section 904, allowing users to view alerts by categories such as “Denials,” “Submissions,” “Collections,” and “Remits.” These filters may enable users to focus on specific aspects of the revenue cycle that are most relevant to their responsibilities. A time period selector displays “All Time” as the current selection, allowing users to adjust the timeframe for the displayed alerts and metrics. In some implementations, the filtering system may incorporate AI-driven recommendations to suggest the most relevant filters based on the current state of the revenue cycle or the user's historical behavior.

The GUI 900 demonstrates an approach to presenting healthcare revenue cycle data and alerts in an intuitive and actionable format. By leveraging AI and machine learning techniques, the system may provide timely and relevant information to users, enabling them to quickly identify and address issues in their revenue cycle management processes. This approach may address the technical challenges of managing complex healthcare financial data, allowing healthcare providers to optimize their operations and improve their financial performance.

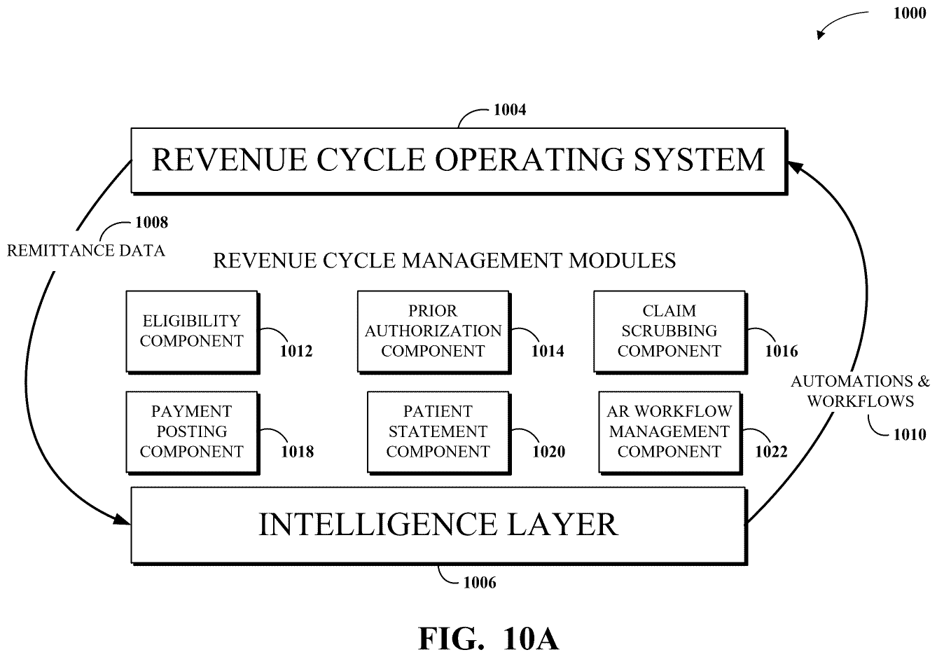

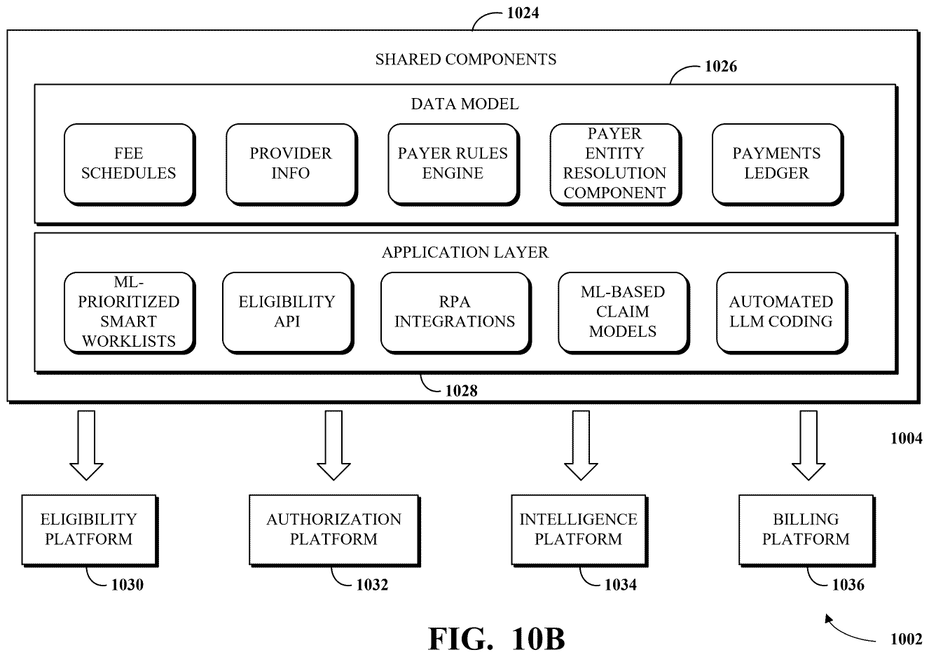

FIGS. 10A and 10B illustrate block diagrams of an AI system 1000 and a system architecture 1002 for processing healthcare revenue cycle data according to aspects of the present disclosure.

FIG. 10A depicts the AI system 1000, which includes an example of an operation cycle operating system (in this case, a revenue cycle operating system 1004) that processes remittance data 1008 and interfaces with an intelligence layer 1006. The revenue cycle operating system 1004 may serve as the core platform for managing and optimizing healthcare revenue cycle processes. In some implementations, the revenue cycle operating system 1004 may be implemented as a cloud-based solution, allowing for scalability and accessibility across multiple healthcare provider locations. In some implementations, the system may be deployed on-premises or in a hybrid cloud configuration, depending on the specific needs and regulatory requirements of healthcare providers.

The revenue cycle operating system 1004 contains multiple revenue cycle management modules arranged in a grid layout. These modules include an eligibility component 1012, which may be responsible for verifying patient insurance eligibility and benefits. In some implementations, the eligibility component 1012 may utilize real-time connectivity to payer systems to provide instant eligibility verification. In some implementations, it may employ a batch processing approach for high-volume eligibility checks during off-peak hours.

A prior authorization component 1014 is included for handling pre-authorizations for medical procedures and treatments. This component may incorporate rule-based engines to determine when prior authorizations are required based on procedure codes, payer policies, and patient information. In some implementations, the prior authorization component 1014 may include machine learning models to predict the likelihood of authorization approval and suggest optimal submission strategies.

The system also includes a claim scrubbing component 1016 for validating claims before submission. This component may employ a combination of rule-based checks and machine learning algorithms to identify potential errors or omissions in claim data. In some implementations, the claim scrubbing component 1016 may integrate with medical coding databases to ensure up-to-date coding validation. In some implementations, it may incorporate natural language processing capabilities to extract relevant information from clinical notes for claim validation.

A payment posting component 1018 is responsible for processing incoming payments and reconciling them with submitted claims. This component may utilize optical character recognition (OCR) and machine learning techniques to automate the ingestion and processing of remittance advice documents. In some implementations, the payment posting component 1018 may include advanced matching algorithms to handle complex payment scenarios, such as bundled payments or partial reimbursements.

The revenue cycle operating system 1004 also features a patient statement component 1020 for managing patient billing documents. This component may generate personalized patient statements based on remaining balances after insurance payments. In some implementations, the patient statement component 1020 may incorporate dynamic content generation capabilities to provide tailored explanations of charges and payment options based on patient demographics and payment history.

An AR workflow management component 1022 is included for accounts receivable processing. This component may utilize machine learning algorithms to prioritize and route accounts for follow-up based on factors such as age, amount, and likelihood of collection. In some implementations, the AR workflow management component 1022 may include predictive analytics capabilities to forecast cash flow and identify accounts at risk of becoming delinquent.

The automations and workflows 1010 connect the various components of the system, enabling data flow between modules. In some implementations, these automations may be implemented using robotic process automation (RPA) techniques to streamline repetitive tasks and reduce manual intervention. In some implementations, the system may employ a microservices architecture to facilitate modular development and deployment of individual workflow components.

The intelligence layer 1006 processes information from the revenue cycle operating system 1004 and provides analytical capabilities. This layer may incorporate various machine learning models, including supervised learning for predictive analytics, unsupervised learning for anomaly detection, and reinforcement learning for optimizing revenue cycle strategies. In some implementations, the intelligence layer 1006 may utilize federated learning techniques to improve model performance across multiple healthcare organizations while maintaining data privacy.

The remittance data 1008 flows between the revenue cycle operating system 1004 and the intelligence layer 1006, creating a feedback loop for continuous processing and analysis. This data may include detailed information about claim adjudication results, payment amounts, and denial reasons. In some implementations, the system may employ advanced data compression techniques to efficiently store and process large volumes of remittance data. In some implementations, it may utilize streaming data processing frameworks to enable real-time analysis of incoming remittance information.

FIG. 10B illustrates a system architecture 1002 for the revenue cycle operating system 1004. The system architecture 1002 includes shared components 1024 and an application layer 1028 that work together to provide revenue cycle management functionality.

The shared components 1024 contain a data model 1026 that includes several elements: fee schedules, provider info, payer rules engine, payer entity resolution component, and payments ledger. These shared components may serve as the foundational data structures and services used across the entire revenue cycle operating system. In some implementations, the data model 1026 may utilize graph database technologies to represent complex relationships between healthcare entities and financial transactions. In some implementations, it may employ a hybrid data storage approach, combining relational databases for structured data with document stores for semi-structured information.

The application layer 1028 includes multiple components that leverage the shared components to deliver specific revenue cycle management functionalities. These components include ML-prioritized smart worklists, which may use machine learning algorithms to optimize task prioritization and assignment based on factors such as claim complexity, dollar amount, and likelihood of successful resolution. In some implementations, these worklists may incorporate natural language processing capabilities to analyze and categorize free-text notes associated with claims or denials.

An eligibility API is included in the application layer 1028, providing programmatic access to insurance eligibility verification services. This API may support both real-time and batch eligibility checks, allowing for flexible integration with various healthcare IT systems. In some implementations, the eligibility API may incorporate caching mechanisms to improve performance for frequently queried patient-payer combinations. In some implementations, the API may be called as part of an automated check responsively performed according to an event such as the receipt of input that triggers the check, a detection of an anomaly that triggers the check, or a prediction of an event that triggers the check, among other examples.

The application layer 1028 also features RPA integrations, which may automate repetitive tasks across different systems and interfaces involved in the revenue cycle. These integrations may include screen scraping capabilities for interacting with legacy systems that lack modern APIs. In some implementations, the RPA integrations may utilize computer vision techniques to navigate complex user interfaces and extract relevant information.

ML-based claim models are included in the application layer 1028, providing predictive capabilities for various aspects of the claims lifecycle. These models may predict the likelihood of claim denials, expected reimbursement amounts, or optimal timing for claim submission. In some implementations, these models may employ ensemble learning techniques, combining multiple algorithms to improve overall prediction accuracy and robustness.

The application layer 1028 also includes automated LLM coding, which may utilize large language models to assist with medical coding tasks. This component may analyze clinical documentation to suggest appropriate diagnosis and procedure codes. In some implementations, the automated LLM coding may incorporate domain-specific pre-training on medical literature and coding guidelines to enhance its performance in the healthcare context.

The system architecture 1002 connects to multiple platform components through directional arrows. These include an eligibility platform 1030, an authorization platform 1032, an intelligence platform 1034, and a billing platform 1036. The platforms are arranged horizontally and receive input from the shared components 1024 and application layer 1028. This modular architecture may allow for flexible deployment and scaling of individual platform components based on specific organizational needs. In some implementations, each platform may be containerized to facilitate easy deployment and management across different computing environments.

The shared components 1024 and application layer 1028 are positioned side by side within the revenue cycle operating system 1004, enabling data and functionality sharing between the components. This architecture may promote code reuse and consistency across different modules of the system. In some implementations, the system may employ a service mesh architecture to manage communication and security between different components, enabling fine-grained control over service-to-service interactions.

The directional arrows indicate the flow of information from the revenue cycle operating system 1004 to each specialized platform. This unidirectional flow may help maintain clear boundaries between different functional areas of the system. In some implementations, the system may incorporate event-driven architectures, using message queues or pub/sub systems to facilitate asynchronous communication between components and improve overall system responsiveness.

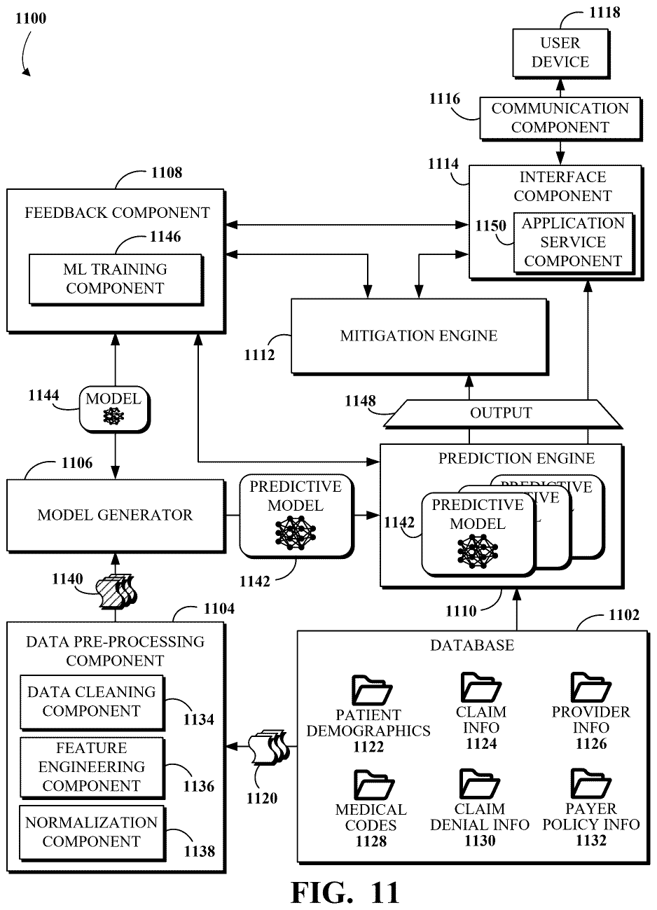

FIG. 11 illustrates a block diagram of an AI system 1100 for processing healthcare claims data according to aspects of the present disclosure. The AI system 1100 comprises multiple interconnected components designed to process, analyze, and optimize healthcare revenue cycle management data. In some implementations, the AI system 1100 may be, be similar to, include, or be included in, the AI system 100 shown in FIG. 1A.

The AI system 1100 includes a database 1102 that stores various types of healthcare-related information. This database 1102 may contain patient demographics 1122, claim information 1124, provider information 1126, medical codes 1128, claim denial information 1130, and payer policy information 1132. In some implementations, the database 1102 may utilize advanced database technologies to efficiently manage large volumes of healthcare data. For example, the database 1102 might employ a combination of relational and NoSQL databases to accommodate the diverse types of data encountered in revenue cycle management. In some implementations, the database 1102 could incorporate a data lake architecture to store and analyze large volumes of unstructured and semi-structured data, providing greater flexibility for future analytics and machine learning applications.

A data preprocessing component 1104 is included in the AI system 1100, responsible for receiving data input 1120 and processing it through several subcomponents. These subcomponents include a data cleaning component 1134, a feature engineering component 1136, and a normalization component 1138. In some implementations, the data preprocessing component 1104 may employ advanced ETL (Extract, Transform, Load) processes, utilizing machine learning algorithms to automate data cleansing and normalization tasks. For instance, the data cleaning component 1134 might use natural language processing techniques to extract relevant information from unstructured clinical notes or payer correspondence.

The data preprocessing component 1104 may employ advanced NLP techniques to extract relevant information from unstructured clinical notes and payer correspondence. In some implementations, the AI system may utilize a combination of named entity recognition (NER) and relation extraction models to identify and categorize key elements within the text. For example, a bidirectional long short-term memory (BiLSTM) network with a conditional random field (CRF) layer may be used to recognize and label entities such as medical conditions, procedures, medications, and dates within clinical notes. This NER model may be pre-trained on large corpora of medical text and fine-tuned on domain-specific datasets to improve accuracy in identifying healthcare-specific entities.

To extract relationships between identified entities, the AI system may employ attention-based transformer models, such as BERT (Bidirectional Encoder Representations from Transformers) or its biomedical variant BioBERT. These models may be used to classify the semantic relationships between entities, such as “medication treats condition” or “procedure performed on date.” In some cases, the AI system may incorporate a graph convolutional network (GCN) to capture complex, multi-hop relationships within the clinical narrative. The extracted structured information may then be mapped to standardized medical codes (e.g., ICD-10, CPT) using a combination of rule-based systems and machine learning classifiers, enabling seamless integration with other structured data in the revenue cycle management process.

The feature engineering component 1136 may employ sophisticated techniques to create relevant features for healthcare revenue cycle prediction tasks. In some implementations, the system may utilize domain-specific knowledge to generate complex features that capture nuanced relationships within the healthcare data. For example, the feature engineering component 1136 may create temporal features that track the progression of a patient's treatment over time, combining information from multiple encounters and procedures. These temporal features may include the time between related procedures, the sequence of diagnoses, or the frequency of certain types of claims for a given patient. In some implementations, the system may generate features that capture the interactions between different medical codes, such as the co-occurrence of specific diagnosis and procedure codes, which may be indicative of certain billing patterns or potential areas for denial.

The normalization component 1138 may implement advanced techniques to ensure consistency across diverse data sources and facilitate accurate analysis. In some cases, the system may employ adaptive normalization strategies that adjust based on the specific characteristics of each data source. For instance, when dealing with laboratory results from different facilities, the normalization component 1138 may use a combination of statistical methods and machine learning algorithms to align different reference ranges and units of measurement. This may involve techniques such as z-score normalization or quantile normalization, with the specific approach selected based on the distribution of the data. Furthermore, the normalization component 1138 may incorporate external reference data, such as standardized medical ontologies or regional healthcare benchmarks, to ensure that normalized values are meaningful and comparable across different healthcare contexts.

In some implementations, the data preprocessing component 1104 may include a data augmentation module that enhances the quality and quantity of training data for machine learning models. This module may employ techniques such as synthetic data generation or data perturbation to create additional training examples that improve model robustness and generalization. For instance, the AI system may use generative adversarial networks (GANs) to create synthetic medical claims that mimic the characteristics of real claims, helping to balance datasets for rare conditions or uncommon billing scenarios. In some implementations, the data augmentation module may apply controlled noise or variations to existing data points, simulating different scenarios such as data entry errors or variations in coding practices. This augmented dataset may help train models that are more resilient to real-world data inconsistencies and variations in healthcare billing practices.

In some implementations, the feature engineering component 1136 could incorporate domain-specific knowledge to create relevant features for healthcare revenue cycle prediction tasks. The feature engineering component 1136 may incorporate domain-specific knowledge to create relevant features for healthcare revenue cycle prediction tasks by leveraging expert insights and industry-specific patterns. In some implementations, the feature engineering component 1136 may utilize a knowledge graph that encapsulates relationships between various healthcare concepts, such as diagnoses, procedures, medications, and billing codes. This knowledge graph may be constructed using medical ontologies, clinical guidelines, and historical billing data to capture complex interactions that influence revenue cycle outcomes.

For example, the feature engineering component 1136 may generate features that reflect the complexity of a patient's case based on the combination of diagnoses and procedures. The feature engineering component 1136 may assign higher complexity scores to cases involving multiple chronic conditions or intricate surgical procedures, as these may be associated with increased likelihood of claim denials or longer processing times. In some implementations, the feature engineering component 1136 may create features that capture the alignment between diagnoses and procedures, flagging potential mismatches that could lead to claim rejections. For instance, the feature engineering component 1136 may generate a “diagnosis-procedure coherence” feature that quantifies how well the documented diagnoses support the billed procedures based on established medical guidelines and historical billing patterns.

In some cases, the feature engineering component 1136 may incorporate temporal aspects of patient care into the generated features. The feature engineering component 1136 may create sequence-based features that capture the progression of treatments over time, such as the order and timing of diagnostic tests, procedures, and follow-up visits. These temporal features may help predict the likelihood of claim denials based on the adherence to expected care pathways or the presence of gaps in documentation. Furthermore, the feature engineering component 1136 may generate features that reflect the historical performance of specific provider-payer combinations, such as average claim processing times or denial rates for particular types of procedures. These provider-payer interaction features may help the AI system account for variations in billing practices and payer policies that can impact revenue cycle outcomes.

The feature engineering component 1136 may also leverage domain-specific knowledge to create features that capture regulatory and compliance aspects of healthcare billing. The feature engineering component 1136 may generate features that reflect the adherence to specific documentation requirements for different types of claims, such as the presence of required modifiers or the completeness of supporting clinical notes. In some implementations, the feature engineering component 1136 may create composite features that combine multiple regulatory factors to produce a “compliance score” for each claim, helping to predict the likelihood of audits or denials based on documentation quality. In some implementations, the feature engineering component 1136 may incorporate external data sources, such as updates to billing guidelines or changes in payer policies, to generate dynamic features that reflect the current regulatory landscape and its potential impact on claim outcomes.

The data preprocessing component 1104 outputs preprocessed data 1140 to a model generator 1106. This model generator 1106 may be responsible for creating a predictive model 1142 based on the preprocessed data. In some implementations, the model generator 1106 may employ various machine learning techniques to create the predictive model, such as logistic regression, random forests, gradient boosting machines, neural networks, or support vector machines. The choice of model may depend on factors such as the specific prediction task, the nature of the available data, and the desired balance between model interpretability and predictive performance.

In some implementations, the AI system may employ advanced pre-processing techniques to enable the identification and prediction of anomalies across data from disparate sources. The feature engineering component 1136 may utilize cross-source feature extraction, where features are derived from multiple data sources simultaneously. For example, the AI system may combine patient demographic information from an electronic health record (EHR) system with claims data from a practice management system to create composite features that capture the relationship between patient characteristics and claim outcomes. This cross-source feature engineering may allow the AI system to identify complex patterns that would not be apparent when analyzing each data source in isolation.

The AI system may also implement a dynamic feature selection process that adapts to changing patterns in healthcare data over time. This process may involve continuously evaluating the relevance and predictive power of features across different data sources. For instance, the AI system may use techniques such as mutual information analysis or recursive feature elimination to periodically reassess which features are most informative for anomaly detection and prediction. This dynamic approach may allow the AI system to maintain high performance even as healthcare regulations, billing practices, and payer policies evolve.

Feedback and retraining mechanisms may play a role in enhancing the AI system's ability to identify and predict anomalies across disparate data sources. The AI system may incorporate a multi-level feedback loop that captures input from various stakeholders in the revenue cycle management process. For example, billing specialists may provide feedback on false positive anomalies, while financial analysts may offer insights on newly emerging patterns. This diverse feedback may be used to fine-tune the anomaly detection models and adjust feature importance weights. In some implementations, the AI system may employ transfer learning techniques to leverage knowledge gained from one healthcare provider or specialty to improve anomaly detection in others, potentially accelerating the learning process for new clients or expanding to new medical domains.

In some implementations, the model generator 1106 may select the appropriate machine learning model based on the specific prediction task and the characteristics of the available data. For example, when predicting the likelihood of claim denials, the AI system may employ a logistic regression model due to its interpretability and ability to provide clear insights into the factors contributing to denials. This approach may be particularly useful when healthcare providers need to understand and explain the reasons behind predicted denials to their staff or patients. In some implementations, for more complex tasks such as predicting expected reimbursement amounts, the system may utilize gradient boosting machines or random forests, which can capture non-linear relationships and interactions between various features in the claims data.

The model selection process may also consider the volume and diversity of available data. In cases where the healthcare organization has a large dataset with many features, the system may opt for deep learning models such as neural networks, which can automatically learn hierarchical representations from the data. These models may be especially effective for tasks like predicting optimal claim submission timing, where complex temporal patterns in historical data may influence outcomes. On the other hand, for smaller datasets or when rapid model development is required, the system may choose simpler models like support vector machines, which can perform well with limited training data and provide faster training times. In some cases, the model generator 1106 may employ automated machine learning (AutoML) techniques to systematically evaluate multiple model types and select the best performing one based on predefined performance metrics such as accuracy, precision, or area under the ROC curve.

The generation of the training data set may involve aggregating historical claims data from multiple healthcare providers across various specialties and geographical regions. This comprehensive dataset may include a wide range of information such as patient demographics, diagnosis codes, procedure codes, claim amounts, payer information, and claim outcomes (e.g., approved, denied, partially paid). The AI system may also incorporate data from electronic health records (EHRs), practice management systems, clearinghouses, and payer portals to enrich the training dataset with additional context and metadata.

In some implementations, the training data set may be augmented with synthetic data generated using techniques such as generative adversarial networks (GANs) or variational autoencoders (VAEs). These synthetic data points may help balance the dataset for rare conditions or uncommon billing scenarios, improving the model's ability to generalize across a wide range of cases. For example, the AI system may generate synthetic claims for rare procedures or complex combinations of diagnoses that are not well-represented in the original dataset, ensuring that the model can handle these scenarios effectively.

The retraining data set may be continuously updated with new claims data as it becomes available, allowing the model to adapt to changing patterns in healthcare billing and payer behavior. This dynamic retraining process may involve a sliding window approach, where the most recent data is given more weight in the training process. For instance, the AI system may prioritize claims data from the past 6-12 months while still retaining some historical context from older data. In some implementations, the retraining data set may incorporate feedback from human billers and revenue cycle managers, capturing information about successful appeals, coding corrections, and other manual interventions that lead to positive claim outcomes.

In some cases, the AI system may employ active learning techniques to identify the most informative samples for retraining. This approach may involve selecting claims that the current model is uncertain about or that represent edge cases in the data distribution. For example, if the model consistently misclassifies certain types of claims or struggles with specific payer-provider combinations, these cases may be prioritized in the retraining data set. The AI system may also leverage the network effect of its user base, identifying trends and patterns across multiple healthcare providers to enhance the retraining process and improve the model's performance across diverse scenarios.

A prediction engine 1110 utilizes the predictive model 1142 generated by the model generator 1106. This prediction engine 1110 may contain multiple instances of the predictive model 1142 and generates prediction output 1148. In some implementations, the prediction engine 1110 may use ensemble methods, combining predictions from multiple models to improve overall accuracy and robustness. In some implementations, the prediction engine 1110 could employ online learning techniques to continuously update the predictive models based on new data, ensuring that the predictions remain relevant in the face of changing healthcare regulations and payer policies.

The prediction engine 1110 may generate predictions using a multi-stage process that incorporates various data sources and machine learning techniques. In some implementations, the system may first analyze historical claim data to identify patterns and trends associated with successful and denied claims. This analysis may include examining factors such as diagnosis codes, procedure codes, patient demographics, provider information, and payer-specific policies. The system may then use these insights to create feature vectors for each new claim, representing the relevant characteristics that influence the likelihood of approval or denial.

In some cases, the prediction engine 1110 may employ a hierarchical approach to generate predictions. For example, it may first use a high-level model to categorize claims into broad risk categories (e.g., low, medium, high risk of denial). Subsequently, specialized models tailored to each risk category may be applied to generate more precise predictions. This approach may allow the system to capture both general trends and nuanced patterns specific to different types of claims or payers.

The prediction engine 1110 may also incorporate real-time data feeds to enhance its predictive capabilities. For instance, it may continuously monitor payer websites and policy updates to detect changes in reimbursement rules or documentation requirements. This information may be used to dynamically adjust prediction models, ensuring that the system remains responsive to evolving healthcare regulations and payer behaviors. In some implementations, the prediction engine may use natural language processing techniques to analyze payer correspondence and automatically extract new rules or policy changes that could affect claim outcomes.

To improve prediction accuracy, the system may leverage network effects by analyzing claim data across multiple healthcare providers. For example, if a particular payer begins denying claims for a specific procedure code more frequently, the system may detect this trend across its network of users and adjust its predictions accordingly for all affected providers. This approach may allow the prediction engine to identify and respond to industry-wide trends more quickly than traditional, siloed systems. In some implementations, the prediction engine may incorporate feedback loops that track the actual outcomes of submitted claims and use this information to continuously refine and improve its predictive models over time.

The AI system may leverage aspects of the machine learning process illustrated in FIG. 3 to aggregate and unify data from multiple sources for predictive modeling in healthcare revenue cycle management. The observation dataset 302 may represent the comprehensive collection of historical claims data, patient demographics, diagnosis codes, procedure codes, and other relevant information gathered from various healthcare providers, electronic health records, practice management systems, and payer portals. This diverse dataset may serve as the foundation for training the predictive models used in revenue cycle management.

In some implementations, the system may apply preprocessing techniques to transform the raw data in the observation dataset 302 into a unified format suitable for machine learning. This process may involve data cleaning, normalization, and feature engineering steps similar to those performed by the data preprocessing component 1104. The resulting preprocessed data may then be used to train a model 306, which could represent various machine learning algorithms such as logistic regression, random forests, or neural networks, depending on the specific prediction task and data characteristics. The trained model 308 may then be employed by the prediction engine 1110 to generate predictions for new input observations 310, which in this context may represent incoming claims or other revenue cycle events requiring analysis.

The system may also incorporate feedback loops and continuous learning mechanisms to refine and improve its predictive capabilities over time. For example, the target variable 314 generated by the trained model for each input observation may be compared to actual outcomes, such as claim approvals or denials. This feedback may be used to update the model through techniques like online learning or periodic retraining, ensuring that the predictive models remain accurate and relevant in the face of changing healthcare regulations and payer behaviors. In some implementations, the AI system may employ unsupervised learning techniques, as represented by the clustering operation 316 in FIG. 3, to identify patterns or groupings in the data that may not be immediately apparent, potentially uncovering new insights into factors affecting revenue cycle outcomes.

The AI system 1100 includes a feedback component 1108 that contains an ML training component 1146. This feedback component 1108 works with a trained model 1144 and interacts with both the model generator 1106 and a mitigation engine 1112 to improve system performance. In some implementations, the feedback component 1108 may employ reinforcement learning techniques to optimize the system's decision-making processes over time. In some implementations, the ML training component 1146 could use transfer learning approaches to adapt pre-trained models to specific healthcare organizations or specialties, allowing for faster model deployment and improved performance on smaller datasets.

The ML training component 1146 may employ a multi-faceted approach to continuously improve the system's predictions. In some implementations, it may utilize a combination of supervised and unsupervised learning techniques to refine the predictive models based on new data and outcomes. For example, as new claims are processed and their outcomes become known, the ML training component may update the model parameters to reflect the latest patterns and trends in claim approvals and denials. This ongoing training process may allow the system to adapt to changes in payer policies, healthcare regulations, and provider practices over time.

In some cases, the ML training component 1146 may implement a federated learning approach to leverage data from multiple healthcare providers while maintaining data privacy and security. This technique may allow the system to learn from a broader dataset without directly sharing sensitive patient or claim information between organizations. For instance, the ML training component may distribute model updates to individual healthcare providers, who then train the model on their local data and return only the updated model parameters. The system may then aggregate these updates to improve the global model, potentially capturing nuanced patterns that may not be apparent in any single provider's data alone.

The ML training component 1146 may also incorporate active learning strategies to efficiently improve model performance in areas of uncertainty. In this approach, the system may identify claims or scenarios where the predictive model has low confidence and prioritize these cases for human review. For example, if the system encounters a new combination of procedure codes and diagnoses that it hasn't seen before, it may flag this claim for manual review by a billing specialist. The outcome of this review may then be used to update the model, improving its ability to handle similar cases in the future. This human-in-the-loop process may be particularly valuable for adapting to new medical procedures, emerging healthcare technologies, or changes in coding practices that may not be well-represented in historical data.

The mitigation engine 1112 processes the output from the prediction engine 1110. In some implementations, this mitigation engine 1112 may be responsible for generating recommendations or automating actions based on the predictions. For example, it might suggest preemptive measures to reduce the likelihood of claim denials or automate the process of addressing certain types of denials. In some implementations, the mitigation engine 1112 could employ rule-based systems in conjunction with machine learning models to ensure that automated actions comply with healthcare regulations and organizational policies.

The mitigation engine 1112 may facilitate various actions to address potential claim denials and optimize the revenue cycle process. In some implementations, the mitigation engine may generate smart worklists that prioritize claims based on their likelihood of denial, expected reimbursement amount, and time sensitivity. These worklists may be dynamically updated as new information becomes available, ensuring that billing staff focus their efforts on the most critical and high-value claims. For example, the system may assign higher priority to claims approaching timely filing deadlines or those with a high predicted probability of denial but also a high potential reimbursement value.

In some cases, the mitigation engine 1112 may provide detailed suggestions on how to prepare or fix claims to increase their chances of approval. These suggestions may include recommendations for additional documentation, coding modifications, or clarifications needed to support the claim. For instance, if the system detects that a particular combination of diagnosis and procedure codes frequently leads to denials for a specific payer, it may suggest alternative coding options or additional supporting documentation to include with the claim submission. The mitigation engine may also leverage natural language processing capabilities to automatically generate appeal letters or documentation requests, tailoring the content based on the specific reasons for potential denial and the payer's known preferences.

The mitigation engine 1112 may also facilitate rule changes and policy updates within the healthcare organization's billing processes. By analyzing patterns in successful and denied claims across multiple providers and payers, the system may identify opportunities to update internal billing guidelines or create new rules to prevent common denial reasons. For example, if the system detects that a particular payer has recently changed its policy regarding pre-authorization requirements for certain procedures, it may suggest updating the organization's claim submission rules to automatically flag claims that require pre-authorization before submission. In some implementations, the mitigation engine may provide insights into the effectiveness of current billing practices, allowing revenue cycle managers to make data-driven decisions about process improvements and resource allocation.

The AI system 1100 interfaces with users through a user device 1118 connected via a communication component 1116. An interface component 1114 manages user interactions, while an application service component 1150 coordinates the various system operations and data flows between components. In some implementations, the interface component 1114 may provide customizable dashboards for different user roles, such as billing specialists, revenue cycle managers, or financial analysts. In some implementations, the application service component 1150 could implement a microservices architecture, allowing for greater flexibility and scalability in deploying and updating individual components of the AI system 1100.

The AI system 1100 demonstrates an approach to leveraging machine learning for healthcare revenue cycle management. For instance, in some implementations, the system obtains a first set of data (e.g., patient demographics 1122, claim information 1124) from the database 1102. The model generator 1106 then creates a machine learning model based on this data, which is trained to become a predictive model 1142. This predictive model is then used by the prediction engine 1110 to determine predicted metrics associated with the revenue cycle, such as the likelihood of claim denials or expected reimbursement amounts.

In some implementations, the data preprocessing component 1104 performs various preprocessing operations on the structured data. These operations may include data cleaning (via component 1134), feature engineering (via component 1136), and normalization (via component 1138). The feature engineering component 1136 may identify a set of features associated with the first set of data, which could include claim amounts, medical codes, payer types, provider types, patient demographics, or statistics associated with historical metrics.

The system's ability to handle different types of machine learning models is evident in the flexibility of the model generator 1106 and prediction engine 1110. These components may work with various model types, including logistic regression, random forests, gradient boosting machines, neural networks, or support vector machines, depending on the specific requirements of the prediction task.

The feedback component 1108 and ML training component 1146 may implement techniques such as using weighted loss functions for imbalanced data, performing resampling operations on minority classes, and retraining the predictive model based on feedback information to continuously improve the system's performance.

In some implementations, the prediction output 1148 generated by the prediction engine 1110 may be used to create user interface elements for presentation on the user device 1118, or to trigger mitigation operations via the mitigation engine 1112. These mitigation operations could include recommendation systems or robotic process automation to address potential issues in the revenue cycle proactively.

By integrating these various components and functionalities, the AI system 1100 provides a comprehensive solution for healthcare revenue cycle management, leveraging machine learning to improve prediction accuracy, streamline workflows, and ultimately optimize financial performance for healthcare providers.

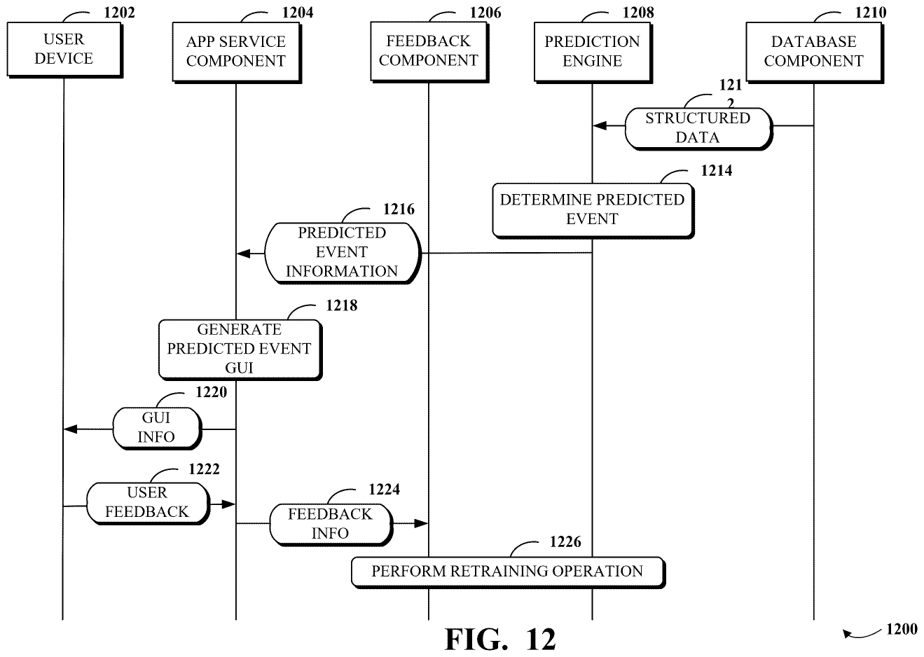

FIG. 12 is a flow diagram of an example of a process 1200 for handling predicted events and user feedback according to aspects of the present disclosure. The process 1200 depicts the flow of data through various components of an AI system for healthcare revenue cycle management.

The process 1200 begins with a user device 1202, which may represent various endpoints through which users interact with the AI system. These devices may include desktop computers, laptops, tablets, smartphones, or specialized healthcare workstations. In some implementations, the user device 1202 may be a wearable device or a voice-activated assistant, allowing for hands-free interaction in clinical settings. For example, a billing specialist might use a desktop computer to review claim predictions, while a physician could use a tablet device to quickly check the status of submitted claims between patient consultations. In some implementations, the user device 1202 could be an augmented reality headset that overlays revenue cycle information onto the user's field of view, enabling seamless integration of financial data into clinical workflows.

An application service component 1204 acts as an intermediary between the user device 1202 and the analytical components of the AI system. This component may manage user authentication, handle API requests, and coordinate the flow of information between different parts of the system. In some implementations, the application service component 1204 may implement a microservices architecture, allowing for greater flexibility and scalability in deploying and updating individual components of the AI system. For instance, separate microservices could be dedicated to handling user authentication, managing API requests, and coordinating data flow, enabling independent scaling and updates of these functionalities. In some implementations, the application service component 1204 might incorporate an event-driven architecture to enable real-time responsiveness to changes in the healthcare revenue cycle landscape, such as immediate updates to prediction models when new payer policies are detected.

The process 1200 includes a feedback component 1206 that may play a role in the continuous improvement of the AI system. This component may capture user interactions, preferences, and manual corrections to refine the system's algorithms and recommendations. In some implementations, the feedback component 1206 may employ both explicit feedback mechanisms (e.g., user ratings or comments) and implicit feedback (e.g., usage patterns or time spent on specific tasks) to enhance the system's performance. For example, if a user frequently overrides certain types of claim predictions, the feedback component 1206 could flag these instances for review and potential model adjustment. In some implementations, the feedback component 1206 might utilize sentiment analysis techniques to gauge user satisfaction and identify areas for improvement based on natural language feedback provided by users.

A prediction engine 1208 represents an analytical capability of the AI system. This engine may leverage various machine learning models to generate predictions about different aspects of the healthcare revenue cycle. In some implementations, the prediction engine 1208 may employ ensemble methods, combining multiple models to improve overall prediction accuracy and robustness. For instance, a random forest classifier might be used in conjunction with a gradient boosting machine to predict the likelihood of claim denials, leveraging the strengths of both algorithms to capture complex patterns in the data. In some implementations, the prediction engine 1208 could utilize deep learning architectures, such as recurrent neural networks or transformer models, to analyze sequential data in the claims processing workflow and predict outcomes like expected time to payment or probability of successful appeals.

The process 1200 includes a database component 1210 that serves as the data storage and retrieval system for the AI platform. This component may utilize advanced database technologies to efficiently manage large volumes of healthcare data. In some implementations, the database component 1210 might employ a combination of relational and NoSQL databases to accommodate the diverse types of data encountered in revenue cycle management. For example, structured claim data might be stored in a relational database for efficient querying, while unstructured clinical notes could be stored in a document-oriented NoSQL database. In some implementations, the database component 1210 could incorporate a data lake architecture to store and analyze large volumes of unstructured and semi-structured data, providing greater flexibility for future analytics and machine learning applications.

The database component 1210 contains structured data 1212, which represents the harmonized and normalized version of the input data from various healthcare IT systems. This structured data may include various types of information relevant to healthcare revenue cycle management, such as patient demographics, insurance details, diagnosis and procedure codes, claim statuses, and payment information. In some implementations, the structured data 1212 could also incorporate derived features or calculated metrics that provide additional insights into revenue cycle performance, such as predicted likelihood of claim denials or estimated time to payment. For instance, the system might generate a “complexity score” for each claim based on factors like the number of procedure codes, the rarity of the diagnosis, and the historical denial rate for similar claims.

The process 1200 involves an operation 1214 performed by the prediction engine 1208 to generate predicted event information 1216 based on the structured data 1212. This operation may involve applying one or more machine learning models to the input data to forecast various aspects of the revenue cycle. In some implementations, the operation 1214 might include multiple stages of prediction, such as first classifying claims into risk categories and then applying specialized models to each category for more precise predictions. For example, the system might first categorize claims as low, medium, or high risk for denial, and then use category-specific models to predict the exact probability of denial and the expected reimbursement amount. In some implementations, the operation 1214 could employ a single end-to-end deep learning model that takes in all available data and simultaneously predicts multiple outcomes, such as denial probability, expected payment amount, and optimal submission timing.

The application service component 1204 receives the predicted event information 1216 and performs operation 1218 to generate GUI info 1220 for display on the user device 1202. This operation may involve transforming the technical prediction results into user-friendly visualizations and actionable insights. In some implementations, operation 1218 might include generating interactive dashboards that allow users to explore the predictions from different angles and drill down into specific details. For instance, a high-level overview might show aggregate predictions for denial rates and expected revenue, with the ability to click through to view predictions for individual claims or providers. In some implementations, operation 1218 could generate natural language summaries of the predictions, using advanced language models to explain the key insights and recommended actions in plain English.

The user device 1202 can provide user feedback 1222 in response to the displayed predictions and insights. This feedback may include actions taken based on the predictions, corrections to inaccurate predictions, or general comments on the system's performance. In some implementations, the user feedback 1222 might be captured through explicit mechanisms like rating scales or feedback forms integrated into the user interface. For example, users might be prompted to rate the accuracy and usefulness of predictions after taking action on a claim. In some implementations, the system could employ implicit feedback mechanisms, such as tracking which predictions users act upon and which they ignore, to infer the perceived value and accuracy of different types of predictions.

The feedback component 1206 processes the user feedback 1222 into feedback information 1224, which can be used to improve the AI system's performance. This processing may involve aggregating feedback from multiple users, identifying patterns or trends in the feedback, and translating user actions into meaningful signals for model improvement. In some implementations, the feedback component 1206 might employ natural language processing techniques to extract insights from free-text feedback comments. For instance, it could identify common themes or sentiments expressed by users about different aspects of the system's predictions. In some implementations, the feedback component 1206 could use reinforcement learning techniques to continuously optimize the system's decision-making processes based on the outcomes of actions taken in response to its predictions.

The process 1200 concludes with operation 1226, where the feedback component 1206 uses the processed feedback information to update and improve the AI system. This operation may involve retraining machine learning models, adjusting prediction thresholds, or refining the rules used for generating insights and recommendations. In some implementations, operation 1226 might include an automated model retraining pipeline that periodically updates the prediction models using the latest feedback and outcomes data. For example, the system could retrain its claim denial prediction model weekly, incorporating the latest feedback and actual claim outcomes to improve its accuracy. In some implementations, operation 1226 could involve a more nuanced approach, where different components of the system are updated at different frequencies or in response to specific triggers, such as detecting a significant shift in prediction accuracy for a particular type of claim.

By implementing this comprehensive process for handling predicted events and user feedback, the AI system can continuously improve its performance and provide increasingly accurate and valuable insights for healthcare revenue cycle management. The iterative nature of the process, combined with the integration of advanced machine learning techniques and user feedback mechanisms, enables the system to adapt to the dynamic healthcare landscape and deliver personalized, actionable intelligence to its users.

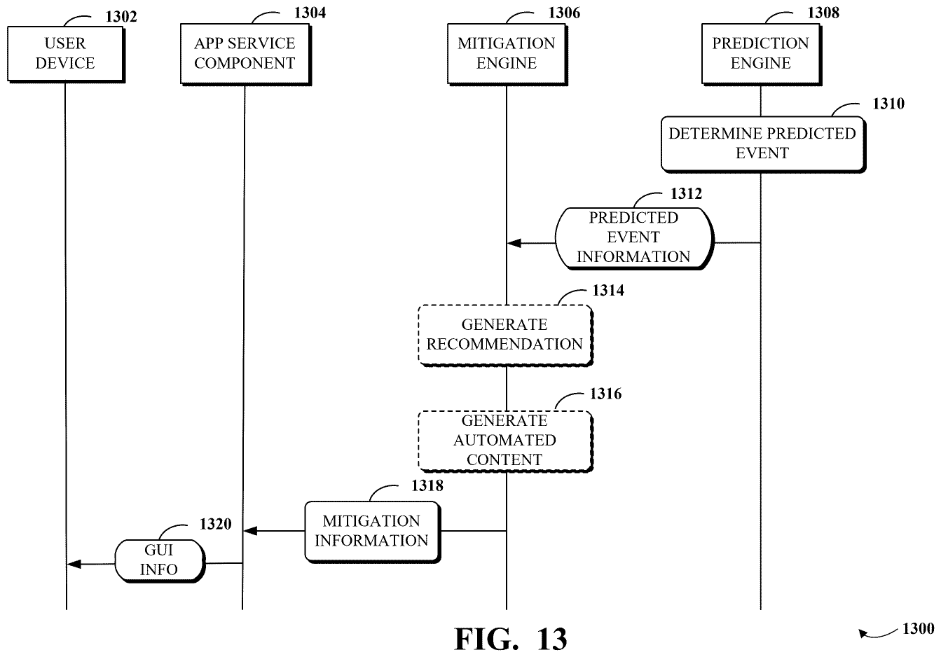

FIG. 13 is a flow diagram of an example of a process 1300 for generating and providing mitigation information according to aspects of the present disclosure. The process 1300 depicts the flow of data through various components of an AI system for healthcare revenue cycle management.

The process 1300 begins with a user device 1302, which may represent various endpoints through which users interact with the AI system. These devices may include desktop computers, laptops, tablets, smartphones, or specialized healthcare workstations. In some implementations, the user device 1302 may be a wearable device or a voice-activated assistant, allowing for hands-free interaction in clinical settings. For example, a billing specialist might use a desktop computer to review claim predictions, while a physician could use a tablet device to quickly check the status of submitted claims between patient consultations. In some implementations, the user device 1302 could be an augmented reality headset that overlays revenue cycle information onto the user's field of view, enabling seamless integration of financial data into clinical workflows.

An application service component 1304 acts as an intermediary between the user device 1302 and the analytical components of the AI system. This component may manage user authentication, handle API requests, and coordinate the flow of information between different parts of the system. In some implementations, the application service component 1304 may implement a microservices architecture, allowing for greater flexibility and scalability in deploying and updating individual components of the AI system. For instance, separate microservices could be dedicated to handling user authentication, managing API requests, and coordinating data flow, enabling independent scaling and updates of these functionalities. In some implementations, the application service component 1304 might incorporate an event-driven architecture to enable real-time responsiveness to changes in the healthcare revenue cycle landscape, such as immediate updates to prediction models when new payer policies are detected.

The process 1300 includes a mitigation engine 1306 that processes information from multiple components and generates mitigation strategies for revenue cycle issues. In some implementations, the mitigation engine 1306 may employ a combination of rule-based systems and machine learning models to generate recommendations for addressing potential claim denials or optimizing billing workflows. For example, the mitigation engine 1306 might suggest preemptive measures to reduce the likelihood of claim denials based on historical patterns and current payer policies. In some implementations, it could automate the process of addressing certain types of denials by generating appeal letters or correcting common coding errors. The mitigation engine 1306 may also prioritize mitigation actions based on factors such as the financial impact of the issue, the likelihood of successful resolution, and available resources.

A prediction engine 1308 works in conjunction with the mitigation engine 1306 to forecast potential issues in the revenue cycle. This engine may leverage various machine learning models to generate predictions about different aspects of healthcare finance. In some implementations, the prediction engine 1308 may employ ensemble methods, combining multiple models to improve overall prediction accuracy and robustness. For instance, a random forest classifier might be used in conjunction with a gradient boosting machine to predict the likelihood of claim denials, leveraging the strengths of both algorithms to capture complex patterns in the data. In some implementations, the prediction engine 1308 could utilize deep learning architectures, such as recurrent neural networks or transformer models, to analyze sequential data in the claims processing workflow and predict outcomes like expected time to payment or probability of successful appeals.

The process 1300 involves an operation 1310 performed by the prediction engine 1308 to generate predicted event information 1312. This operation may involve applying one or more machine learning models to input data to forecast various aspects of the revenue cycle. In some implementations, the operation 1310 might include multiple stages of prediction, such as first classifying claims into risk categories and then applying specialized models to each category for more precise predictions. For example, the system might first categorize claims as low, medium, or high risk for denial, and then use category-specific models to predict the exact probability of denial and the expected reimbursement amount. In some implementations, the operation 1310 could employ a single end-to-end deep learning model that takes in all available data and simultaneously predicts multiple outcomes, such as denial probability, expected payment amount, and optimal submission timing.

The predicted event information 1312 generated by the prediction engine 1308 serves as input for subsequent mitigation operations. This information may include various types of predictions relevant to healthcare revenue cycle management, such as the likelihood of claim denials, expected reimbursement amounts, or potential bottlenecks in the billing process. In some implementations, the predicted event information 1312 might also include confidence scores or uncertainty estimates associated with each prediction, allowing downstream components to make more informed decisions. For example, the system might provide a range of possible outcomes for a claim, along with the probability of each outcome, rather than a single point estimate. In some implementations, the predicted event information 1312 could include explanations or interpretations of the predictions, using techniques like SHAP (SHapley Additive exPlanations) values to identify the key factors influencing each prediction.

Operation 1314 represents the process of generating a recommendation based on the predicted event information 1312. This operation may involve analyzing the predictions and determining the most appropriate course of action to mitigate potential issues or optimize revenue cycle performance. In some implementations, operation 1314 might employ a decision tree or rule-based system to map different types of predictions to specific recommendations. For example, if the predicted probability of claim denial exceeds a certain threshold, the system might recommend additional documentation or pre-submission review. In some implementations, operation 1314 could use a machine learning model trained on historical data to learn the most effective mitigation strategies for different scenarios, allowing the system to adapt its recommendations over time based on observed outcomes.

Operation 1316 involves generating automated content based on the recommendation produced by operation 1314. This step may include creating various types of content to support the implementation of the recommended mitigation strategies. In some implementations, operation 1316 might use natural language generation techniques to produce human-readable explanations of the recommendations, tailored to different user roles within the healthcare organization. For example, it could generate a detailed technical explanation for billing specialists, along with a high-level summary for executives. In some implementations, operation 1316 could produce automated scripts or workflows for robotic process automation systems, enabling the direct implementation of certain mitigation actions without manual intervention.

The mitigation information 1318 produced by these operations represents the final output of the mitigation process. This information may include a combination of recommendations, supporting data, and automated content designed to address potential revenue cycle issues identified by the prediction engine. In some implementations, the mitigation information 1318 might be structured as a prioritized action plan, with specific steps and timelines for addressing each identified issue. In some implementations, it could be presented as a interactive dashboard that allows users to explore different mitigation scenarios and their potential impacts on revenue cycle performance.

The process 1300 concludes with the generation of GUI info 1320, which transforms the mitigation information into a format suitable for presentation on the user device 1302. This step may involve creating various visual elements, such as charts, graphs, or interactive widgets, to effectively communicate the mitigation strategies and their potential impacts. In some implementations, the GUI info 1320 might be customized based on user roles or preferences, presenting different levels of detail or focusing on specific aspects of the mitigation plan depending on the intended audience. For example, a CFO might see a high-level overview of potential revenue impacts, while a billing specialist would receive detailed instructions for implementing specific mitigation actions. In some implementations, the GUI info 1320 could incorporate augmented reality elements, allowing users to interact with virtual representations of revenue cycle data in their physical environment.

By implementing this comprehensive process for generating and providing mitigation information, the AI system can help healthcare providers proactively address potential revenue cycle issues and optimize their financial performance. The combination of advanced prediction capabilities, intelligent recommendation generation, and user-friendly presentation of mitigation strategies enables healthcare organizations to make data-driven decisions and implement effective solutions to complex revenue cycle challenges.

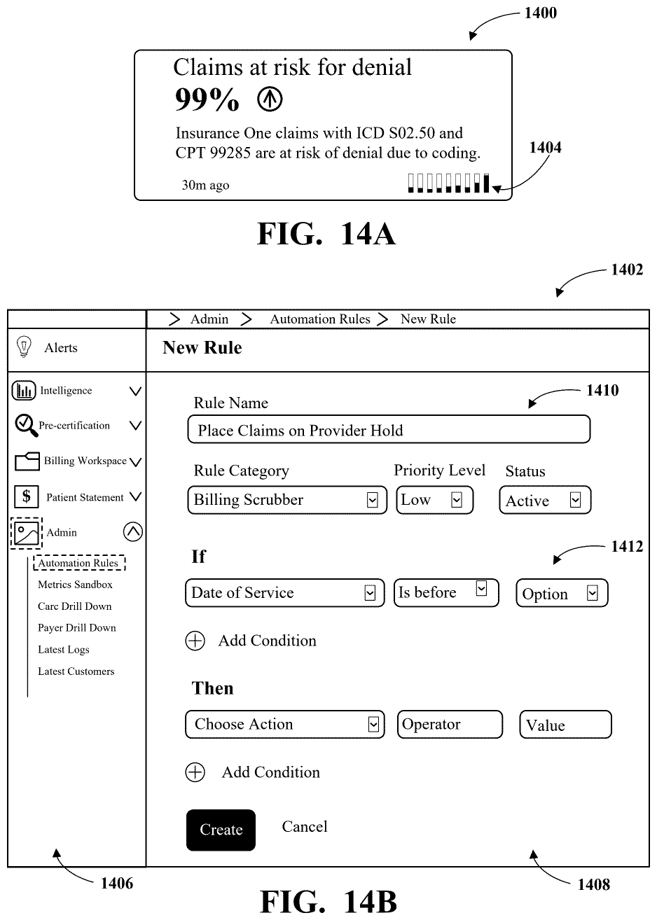

FIGS. 14A and 14B illustrate interfaces of a healthcare claims processing system according to aspects of the present disclosure. FIG. 14A shows a predictive denial interface 1400 that displays claim denial risk information. The interface includes a sparkline 1404 that provides a visual representation of claim denial trends over time. In some implementations, the sparkline 1404 may be interactive, allowing users to hover over data points for more detailed information or select specific time ranges for further analysis. In some implementations, the sparkline 1404 may be color-coded to indicate positive or negative trends, or include markers for significant events or threshold crossings.

FIG. 14B shows a mitigation interface 1402 for configuring automated rules to handle claims. The interface includes a navigation menu 1406 on the left side displaying options like Alerts, Intelligence, Pre-certification, Billing Workspace, Patient Statement, and Admin sections. In some implementations, the navigation menu 1406 may be collapsible to provide more screen space for data visualization. Some implementations may include additional menu items or allow for customization of the menu based on user preferences or role-specific access rights.

The content panel 1408 on the right displays the rule configuration options. Within the content panel 1408, a rule input interface 1410 allows users to specify rule parameters including rule name, category, priority level, and status. In some implementations, the rule input interface 1410 may incorporate machine learning algorithms to suggest optimal rule parameters based on historical data and system performance. In some implementations, the interface may include a template library of pre-configured rules that users can customize for their specific needs.

Below the rule input interface 1410, a condition interface 1412 enables users to set conditions and actions for the rule using dropdown menus and input fields. In some implementations, the condition interface 1412 may utilize natural language processing to allow users to input conditions in plain language, which the system then translates into formal rule logic. Some implementations may include a visual rule builder that allows users to construct complex conditions through a drag-and-drop interface.

The predictive denial interface 1400 and mitigation interface 1402 work together to enable monitoring of claim denial risks and configuration of automated mitigation rules. In some implementations, the system may employ machine learning models to analyze patterns in historical claim data and predict the likelihood of denials for new claims. These predictions may be used to populate the sparkline 1404 and inform the rule creation process in the mitigation interface 1402.

The interfaces may be designed to provide quick, at-a-glance information about specific issues or trends in healthcare revenue cycle management. In some implementations, the system may use natural language generation techniques to create human-readable descriptions of detected anomalies or trends, clearly communicating the issue and its implications to users. Some implementations may incorporate augmented reality features to provide immersive data exploration experiences for healthcare finance professionals.

The system may implement a feedback mechanism to continuously improve its performance based on user interactions and outcomes. In some implementations, the interfaces may include options for users to provide explicit feedback on the accuracy and usefulness of predictions or generated rules. This feedback may be used to refine the underlying machine learning models and improve the system's accuracy over time.

By leveraging artificial intelligence and machine learning techniques throughout the claims processing workflow, the system may address the technical challenges of identifying and responding to revenue cycle issues in a complex and dynamic healthcare environment. The combination of predictive analytics and automated rule configuration may enable healthcare providers to proactively address potential claim denials, optimize their financial operations, and reduce revenue leakage. This approach may allow for more efficient allocation of resources, as billing staff can focus on high-risk claims identified by the system rather than manually reviewing all claims.

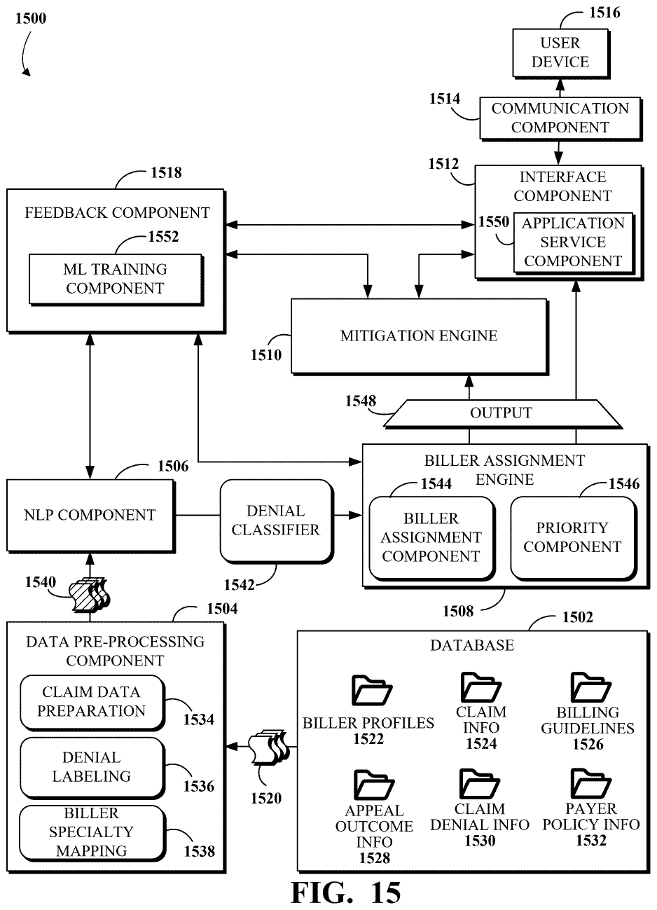

FIG. 15 illustrates a block diagram of an AI system 1500 for processing and managing medical billing information according to aspects of the present disclosure. The AI system 1500 comprises multiple interconnected components designed to process, analyze, and optimize healthcare revenue cycle management data.

The AI system 1500 includes a database 1502 that stores various types of healthcare-related information. This database 1502 may contain patient demographics 1122, claim information 1124, provider information 1126, medical codes 1128, claim denial information 1130, and payer policy information 1132. In some implementations, the database 1502 may utilize advanced database technologies to efficiently manage large volumes of healthcare data. For example, the database 1502 might employ a combination of relational and NoSQL databases to accommodate the diverse types of data encountered in revenue cycle management. In some implementations, the database 1502 could incorporate a data lake architecture to store and analyze large volumes of unstructured and semi-structured data, providing greater flexibility for future analytics and machine learning applications.

A data pre-processing component 1504 is included in the AI system 1500, responsible for receiving data input 1520 and processing it through several subcomponents. These subcomponents include a claim data preparation component 1534, a denial labeling component 1536, and a biller specialty mapping component 1538. In some implementations, the data pre-processing component 1504 may employ advanced ETL (Extract, Transform, Load) processes, utilizing machine learning algorithms to automate data cleansing and normalization tasks. For instance, the claim data preparation component 1534 might use natural language processing techniques to extract relevant information from unstructured clinical notes or payer correspondence. In some implementations, the denial labeling component 1536 could incorporate domain-specific knowledge to create standardized categories for claim denials, improving the consistency and interpretability of the data.

The data pre-processing component 1504 may perform a variety of operations to prepare the raw input data for further analysis and processing. In some implementations, the claim data preparation component 1534 may utilize advanced text parsing algorithms to extract structured information from unstructured claim documents, such as scanned CMS-1500 forms or electronic remittance advice (ERA) files. This component may employ optical character recognition (OCR) techniques to digitize paper-based claims and convert them into machine-readable formats. In some implementations, the claim data preparation component 1534 may implement data validation rules to identify and flag potential errors or inconsistencies in the claim data, such as mismatched diagnosis and procedure codes, invalid patient identifiers, or missing required fields.

The denial labeling component 1536 may leverage machine learning algorithms to automatically categorize claim denials based on their underlying causes. In some aspects, this component may analyze denial reason codes, remittance advice remark codes (RARCs), and free-text explanations provided by payers to assign standardized labels to each denied claim. The component may utilize a combination of supervised and unsupervised learning techniques to identify patterns and clusters in denial data, allowing for the discovery of new denial categories that may not be apparent through manual analysis. Furthermore, the biller specialty mapping component 1538 may use natural language processing and entity recognition techniques to extract information about healthcare providers'specialties and areas of expertise from various data sources, including provider directories, credentialing databases, and historical billing records. This information may be used to create a comprehensive mapping of biller specialties, enabling more efficient assignment of claims to appropriate billing specialists based on their expertise and experience with specific types of claims or payers.

The data pre-processing component 1504 outputs pre-processed data 1540 to an NLP component 1506. This NLP component 1506 may be responsible for analyzing and interpreting human language within healthcare workflows. In some implementations, the NLP component 1506 may employ advanced techniques such as named entity recognition, sentiment analysis, and topic modeling to extract meaningful insights from textual data like claim codes, denial messages, and clinical documentation. For example, the NLP component 1506 might analyze denial reason descriptions to identify common themes or patterns that could inform process improvements. In some implementations, it could be used to interpret and categorize free-text notes from healthcare providers, linking them to specific claims or diagnoses.

The NLP component 1506 may include multiple specialized NLP models, each designed to handle specific aspects of healthcare revenue cycle management. In some implementations, the component may utilize a combination of rule-based and machine learning-based models to process and analyze textual data from various sources. These models may work in parallel or in sequence, depending on the complexity of the task and the nature of the input data. For example, one model may focus on extracting structured information from semi-structured documents like claim forms, while another may specialize in analyzing free-text clinical notes for relevant billing information.

The NLP component 1506 may employ advanced deep learning architectures, such as transformer models or bidirectional long short-term memory (BiLSTM) networks, to capture complex linguistic patterns and contextual information in healthcare-related text. These models may be pre-trained on large corpora of medical literature and healthcare documentation, then fine-tuned on domain-specific datasets to improve their performance on revenue cycle management tasks. In some cases, the NLP component may utilize transfer learning techniques to adapt high-performing general-purpose language models to the specialized vocabulary and syntax commonly found in medical billing and coding documents.

To enhance its capabilities, the NLP component 1506 may incorporate domain-specific knowledge bases and ontologies, such as the Unified Medical Language System (UMLS) or SNOMED CT. These resources may help the NLP models accurately interpret medical terminology, recognize relationships between different concepts, and map free-text descriptions to standardized codes and categories. In some implementations, the NLP component may include modules for handling multilingual input, allowing it to process claims and documentation in various languages and standardize the output for consistent analysis across the AI system 1500. The component may also feature adaptive learning mechanisms that enable it to continuously improve its performance based on feedback from human experts and the outcomes of processed claims.

The NLP component 1506 may employ a multi-stage approach to determine reasons for claim denials. In some implementations, the component may first utilize a named entity recognition model to identify key elements within denial messages, such as procedure codes, diagnosis codes, dates of service, and specific medical terms. This extracted information may then be passed through a classification model that categorizes the denial into predefined categories such as “lack of medical necessity,” “incorrect coding,” or “missing documentation.” The classification model may be trained on a large dataset of historical denials and their associated reasons, allowing it to recognize patterns and nuances in denial language across different payers and claim types.

In some cases, the NLP component 1506 may incorporate a semantic parsing module to analyze the syntactic structure of denial messages and extract causal relationships. This module may identify phrases indicating causality, such as “due to,” “because of,” or “resulting from,” and link them to specific elements of the claim. For example, it may determine that a denial is due to a mismatch between the procedure code and the patient's diagnosis, or because a required pre-authorization was not obtained. In some implementations, the NLP component may utilize a sentiment analysis model to gauge the severity or finality of the denial, distinguishing between outright rejections and requests for additional information. This nuanced understanding of denial reasons may enable the AI system 1500 to generate more targeted and effective strategies for appealing or preventing future denials.

The AI system 1500 includes a denial classifier 1542 that processes the output from the NLP component 1506. In some implementations, this denial classifier 1542 may employ various machine learning techniques to categorize claim denials based on their underlying causes or characteristics. For example, it might use a combination of supervised and unsupervised learning approaches to identify patterns in denial data that may not be immediately apparent to human analysts. The denial classifier 1542 could potentially employ ensemble methods, combining multiple models to improve overall classification accuracy and robustness. In some implementations, it might utilize deep learning architectures to capture complex relationships between different features of denied claims.

The denial classifier 1542 may employ a hierarchical classification approach to categorize claim denials with increasing levels of granularity. At the highest level, the classifier may distinguish between technical denials and clinical denials. Technical denials may include issues such as missing information, incorrect patient demographics, or invalid insurance information. Clinical denials, on the other hand, may involve more complex issues related to medical necessity, level of care, or coding accuracy.

Within each of these broad categories, the denial classifier 1542 may further categorize denials into more specific subcategories. For example, technical denials may be classified into subcategories such as “incomplete claim form,” “missing prior authorization,” or “invalid provider credentials.” Clinical denials may be subcategorized into “lack of medical necessity,” “experimental or investigational treatment,” or “non-covered service.” This multi-level classification approach may allow healthcare providers to identify patterns and trends in denial reasons at various levels of detail, enabling targeted interventions and process improvements.

The denial classifier 1542 may also incorporate temporal and contextual information to enhance its classification accuracy. For instance, it may consider the timing of the claim submission relative to the date of service, the patient's insurance coverage period, or the effective dates of specific payer policies. This temporal awareness may help distinguish between denials due to timely filing issues and those related to retroactive policy changes. In some implementations, the classifier may take into account the context of the claim, such as the patient's medical history, concurrent treatments, or the specific healthcare setting (e.g., inpatient, outpatient, emergency). By considering these contextual factors, the classifier may more accurately categorize denials that stem from complex clinical scenarios or unique patient circumstances.

In some implementations, the denial classifier 1542 may utilize a multi-modal approach, combining textual analysis of denial reasons with structured data analysis. For example, it may analyze the denial reason code in conjunction with the associated claim data, such as diagnosis codes, procedure codes, and billed amounts. This multi-modal analysis may enable the classifier to detect subtle patterns and relationships between claim characteristics and denial reasons. For instance, it may identify that a particular combination of diagnosis and procedure codes frequently results in denials for lack of medical necessity from a specific payer, even when the individual codes themselves do not typically trigger such denials. This level of insight may allow healthcare providers to proactively address potential denial risks and optimize their coding and documentation practices.

A biller assignment engine 1508 is a component of the AI system 1500, responsible for allocating work to billing specialists based on various factors. The biller assignment engine 1508 contains a biller assignment component 1544 and a priority component 1546, which work together to generate output 1548. In some implementations, the biller assignment component 1544 may use machine learning algorithms to match the skills and expertise of individual billers with the specific requirements of each claim or denial. For example, it might consider factors such as a biller's historical performance with certain types of claims or payers when making assignments. The priority component 1546 could employ predictive analytics to assess the urgency and potential value of different claims, ensuring that high-priority items are addressed promptly.

The biller assignment engine 1508 may implement a sophisticated process for assigning billers to specific claims or denials. In some implementations, the engine may utilize a multi-factor scoring system that considers various aspects of both the claim and the biller's expertise. For each claim, the system may generate a complexity score based on factors such as the denial reason, the payer involved, the specialty area of the claim, and the potential financial impact. Similarly, each biller may have a profile that includes their areas of expertise, historical performance metrics, current workload, and specific payer knowledge. The biller assignment component 1544 may then use a matching algorithm to align the complexity score of the claim with the most suitable biller profile, aiming to optimize the likelihood of successful resolution.

In some implementations, the biller assignment engine 1508 may leverage a comprehensive historical analysis of each biller's performance to refine the assignment process. The system may track and analyze various metrics for each biller, such as their success rate in overturning denials, average time to resolution, and efficiency in handling specific types of claims or payers. This historical data may be used to create a dynamic expertise profile for each biller, which may be continuously updated based on their ongoing performance. The biller assignment component 1544 may utilize machine learning algorithms to identify patterns in the biller's success rates across different claim types, denial reasons, and payers. For example, the system may recognize that a particular biller has a high success rate in resolving denials related to medical necessity for orthopedic procedures, or that another biller is particularly efficient in handling claims from a specific insurance provider. The priority component 1546 may factor in these expertise profiles when determining the urgency and potential value of assignments, potentially prioritizing high-value or time-sensitive claims for billers with the most relevant expertise. In some implementations, the system may consider the biller's current workload and recent performance trends to ensure a balanced distribution of work and to avoid overburdening high-performing billers. By continuously refining these assignments based on real-time performance data, the biller assignment engine 1508 may optimize the overall efficiency and success rate of the billing team, potentially leading to improved revenue recovery and reduced denial rates.

For example, if a claim is denied due to a complex medical necessity issue in cardiology, the system may assign it to a biller with a strong track record in appealing similar denials and specific expertise in cardiovascular billing codes. The priority component 1546 may further refine this assignment by considering time-sensitive factors. For instance, claims approaching timely filing deadlines or those with high dollar amounts may be given higher priority in the assignment queue. The system may also incorporate load balancing features to ensure an equitable distribution of work among billers, preventing bottlenecks and maintaining overall team efficiency. In some cases, the biller assignment engine 1508 may employ machine learning techniques to continuously refine its assignment algorithms based on the outcomes of previous assignments, adapting to changing patterns in denial types, payer behaviors, and individual biller performance over time.

The AI system 1500 includes a mitigation engine 1510 that processes information from multiple components to generate strategies for addressing revenue cycle issues. In some implementations, the mitigation engine 1510 may employ a combination of rule-based systems and machine learning models to generate recommendations for addressing potential claim denials or optimizing billing workflows. For example, it might suggest preemptive measures to reduce the likelihood of claim denials based on historical patterns and current payer policies. In some implementations, it could automate the process of addressing certain types of denials by generating appeal letters or correcting common coding errors.

The mitigation engine 1510 may implement a smart worklist system to optimize the workflow for addressing claim denials and other revenue cycle issues. This system may dynamically prioritize and organize tasks based on various factors such as the likelihood of successful appeal, potential financial impact, and approaching deadlines. For instance, the smart worklist may place high-priority denials related to medical necessity at the top of the list, especially if they are from payers known to have strict appeal deadlines. The system may also group similar denials together, allowing billers to efficiently address multiple related issues in a single session.

In some implementations, the mitigation engine 1510 may incorporate a sophisticated status tracking mechanism to monitor the progress of denial processing and appeal efforts. This tracking system may provide real-time updates on the status of each claim, including information such as the current stage in the appeal process, any additional documentation required, and the expected resolution date. For example, the system may automatically flag claims that have been in a particular status for an extended period, prompting supervisors to investigate potential bottlenecks or issues. This granular tracking may enable healthcare providers to maintain a clear overview of their entire denial management process and identify areas for improvement.

The mitigation engine 1510 may also leverage machine learning algorithms to generate customized appeal strategies for different types of denials. By analyzing historical data on successful appeals, the system may identify the most effective arguments, supporting documentation, and appeal formats for specific payers and denial reasons. For instance, when addressing a denial for lack of medical necessity from a particular insurance company, the system may suggest including specific clinical guidelines or peer-reviewed studies that have previously led to successful appeals. This data-driven approach may significantly increase the likelihood of overturning denials and recovering lost revenue.

Furthermore, the mitigation engine 1510 may implement a predictive analytics component to forecast potential denials before they occur. This proactive approach may allow healthcare providers to address issues preemptively, reducing the overall denial rate. For example, the system may analyze patterns in historical denials and identify claims with similar characteristics that are at high risk of being denied. It may then generate alerts or recommendations for additional documentation or coding review before the claim is submitted. In some cases, the system may even suggest alternative coding or billing strategies that have a higher likelihood of approval based on the specific payer's policies and historical claim adjudication patterns.

The system interfaces with users through a user device 1516 connected via a communication component 1514. An interface component 1512 manages user interactions, facilitating the presentation of insights and functionalities through intuitive dashboards, reports, and interactive visualizations. In some implementations, the interface component 1512 may provide customizable dashboards for different user roles, such as billing specialists, revenue cycle managers, or financial analysts. In some implementations, it could incorporate augmented reality features to provide immersive data exploration experiences for healthcare finance professionals.

An application service component 1550 coordinates the various system operations and data flows between components. In some implementations, the application service component 1550 could implement a microservices architecture, allowing for greater flexibility and scalability in deploying and updating individual components of the AI system 1500. In some implementations, it might incorporate an event-driven architecture to enable real-time responsiveness to changes in the healthcare revenue cycle landscape, such as immediate updates to prediction models when new payer policies are detected.

The AI system 1500 includes a feedback component 1518 that contains an ML training component 1552. This feedback component 1518 plays a role in the continuous improvement of the system's performance. In some implementations, the feedback component 1518 may employ reinforcement learning techniques to optimize the system's decision-making processes over time. For example, it might track the outcomes of biller assignments or mitigation strategies and use this information to refine the models used by the biller assignment engine 1508 and mitigation engine 1510. In some implementations, the ML training component 1552 could use transfer learning approaches to adapt pre-trained models to specific healthcare organizations or specialties, allowing for faster model deployment and improved performance on smaller datasets.

The ML training component 1552 may employ reinforcement learning techniques to continuously improve the biller assignment and prioritization processes within the AI system 1500. In some implementations, the reinforcement learning model may be structured as a Markov Decision Process (MDP), where the state represents the current status of all claims and billers, actions correspond to assignment decisions, and rewards are based on successful claim resolutions and efficient resource utilization. The system may use algorithms such as Q-learning or Deep Q-Networks (DQN) to learn optimal assignment strategies over time.

In the context of biller assignment, the reinforcement learning model may learn to match billers with claims in a way that maximizes the overall success rate of claim resolutions. The state space may include features such as claim characteristics, biller expertise profiles, current workload, and historical performance data. Actions may involve assigning a specific claim to a particular biller or adjusting the priority of a claim in the worklist. The reward function may be designed to balance multiple objectives, such as maximizing the number of successfully resolved claims, minimizing the time to resolution, and maintaining an equitable workload distribution among billers.

The priority component 1546 may utilize reinforcement learning to dynamically adjust the prioritization of claims in the worklist. In this scenario, the state space may include information about claim deadlines, potential financial impact, likelihood of successful appeal, and current system workload. Actions may involve reordering claims in the worklist or allocating additional resources to high-priority items. The reward function may be structured to incentivize the system to address high-value and time-sensitive claims promptly while also considering the overall efficiency of the revenue cycle management process.

As the system interacts with the environment and receives feedback on the outcomes of its decisions, it may continuously update its policy to improve future assignments and prioritizations. For example, if the system observes that certain types of claims are consistently resolved more quickly when assigned to billers with specific expertise profiles, it may adjust its policy to favor such assignments in the future. Similarly, if the system learns that prioritizing certain types of denials leads to higher success rates in appeals, it may adapt its prioritization strategy accordingly. This adaptive approach may allow the AI system 1500 to optimize its performance over time, potentially leading to improved revenue recovery rates and more efficient utilization of billing resources.

The data input 1520 received by the AI system 1500 may encompass a wide range of healthcare and financial information. This may include, but is not limited to, electronic health records, claims data, remittance advice, and payer policies. In some implementations, the system may incorporate real-time data streams from connected medical devices or wearables to provide a more comprehensive view of patient health and associated billing implications. In some implementations, it might integrate data from external sources such as public health databases or economic indicators to provide additional context for revenue cycle management decisions.

By leveraging these various components and functionalities, the AI system 1500 provides a comprehensive solution for healthcare revenue cycle management. The system's ability to process and analyze large volumes of complex healthcare data, combined with its machine learning capabilities, enables it to identify patterns, predict outcomes, and generate actionable insights that may not be apparent through traditional analysis methods. This approach may address the technical challenges of managing complex healthcare financial data, allowing healthcare providers to optimize their operations, reduce claim denials, and ultimately improve their financial performance.

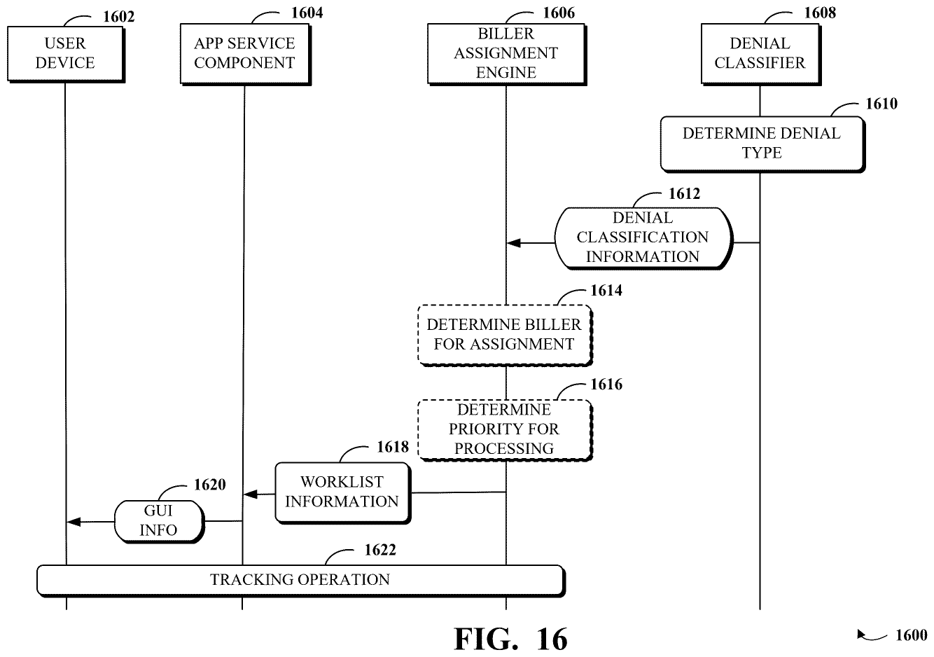

FIG. 16 is a flow diagram of an example of a process 1600 for assigning and tracking medical billing claims according to aspects of the present disclosure. The process 1600 depicts the flow of data through various components of an AI system for healthcare revenue cycle management.

The process 1600 begins with a user device 1602, which may represent various endpoints through which users interact with the AI system. These devices may include desktop computers, laptops, tablets, smartphones, or specialized healthcare workstations. In some implementations, the user device 1602 may be a wearable device or a voice-activated assistant, allowing for hands-free interaction in clinical settings. For example, a billing specialist might use a desktop computer to review claim predictions, while a physician could use a tablet device to quickly check the status of submitted claims between patient consultations. In some implementations, the user device 1602 could be an augmented reality headset that overlays revenue cycle information onto the user's field of view, enabling seamless integration of financial data into clinical workflows.

An application service component 1604 acts as an intermediary between the user device 1602 and the analytical components of the AI system. This component may manage user authentication, handle API requests, and coordinate the flow of information between different parts of the system. In some implementations, the application service component 1604 may implement a microservices architecture, allowing for greater flexibility and scalability in deploying and updating individual components of the AI system. For instance, separate microservices could be dedicated to handling user authentication, managing API requests, and coordinating data flow, enabling independent scaling and updates of these functionalities. In some implementations, the application service component 1604 might incorporate an event-driven architecture to enable real-time responsiveness to changes in the healthcare revenue cycle landscape, such as immediate updates to prediction models when new payer policies are detected.

The process 1600 includes a denial classifier 1608 that processes and categorizes claim denials. In some implementations, this denial classifier 1608 may employ various machine learning techniques to categorize claim denials based on their underlying causes or characteristics. For example, it might use a combination of supervised and unsupervised learning approaches to identify patterns in denial data that may not be immediately apparent to human analysts. The denial classifier 1608 could potentially employ ensemble methods, combining multiple models to improve overall classification accuracy and robustness. In some implementations, it might utilize deep learning architectures to capture complex relationships between different features of denied claims.

A biller assignment engine 1606 is a component of the AI system responsible for allocating work to billing specialists based on various factors. In some implementations, the biller assignment engine 1606 may use machine learning algorithms to match the skills and expertise of individual billers with the specific requirements of each claim or denial. For example, it might consider factors such as a biller's historical performance with certain types of claims or payers when making assignments. The biller assignment engine 1606 could employ predictive analytics to assess the urgency and potential value of different claims, ensuring that high-priority items are addressed promptly.

The process 1600 involves an operation 1610 performed by the denial classifier 1608 to generate denial classification information 1612. This operation may involve applying one or more machine learning models to input data to categorize various aspects of claim denials. In some implementations, the operation 1610 might include multiple stages of classification, such as first categorizing denials into broad categories and then applying specialized models to each category for more precise classification. For example, the system might first categorize denials as technical or clinical, and then use category-specific models to determine the exact reason for denial, such as missing information, coding errors, or lack of medical necessity.

The denial classification information 1612 generated by the denial classifier 1608 serves as input for subsequent assignment operations. This information may include various types of classifications relevant to healthcare revenue cycle management, such as the type of denial, the complexity of the denial, or the estimated effort required to resolve the denial. In some implementations, the denial classification information 1612 might also include confidence scores or uncertainty estimates associated with each classification, allowing downstream components to make more informed decisions. For example, the system might provide a range of possible classifications for a denial, along with the probability of each classification, rather than a single definitive categorization.

Operation 1614 represents the process of assigning billers based on the denial classification information 1612. This operation may involve analyzing the classifications and determining the most appropriate biller to handle each denied claim. In some implementations, operation 1614 might employ a decision tree or rule-based system to map different types of denials to specific billers based on their expertise and workload. For example, if a denial is classified as a complex medical necessity issue, the system might assign it to a biller with extensive experience in that area. In some implementations, operation 1614 could use a machine learning model trained on historical data to learn the most effective biller assignments for different scenarios, allowing the system to adapt its assignments over time based on observed outcomes.

Operation 1616 involves generating worklist information 1618 based on the biller assignments. This step may include creating prioritized task lists for each biller, incorporating factors such as claim value, urgency, and complexity. In some implementations, operation 1616 might use natural language generation techniques to produce human-readable descriptions of each task, tailored to the assigned biller's preferences or expertise level. For example, it could generate detailed instructions for newer billers, while providing more concise summaries for experienced staff. In some implementations, operation 1616 could produce automated scripts or workflows for robotic process automation systems, enabling the direct implementation of certain billing tasks without manual intervention.

The worklist information 1618 produced by these operations represents the final output of the assignment process. This information may include a combination of task descriptions, supporting data, and prioritization details designed to optimize the workflow of billing staff. In some implementations, the worklist information 1618 might be structured as a prioritized action plan, with specific steps and timelines for addressing each assigned claim. In some implementations, it could be presented as an interactive dashboard that allows billers to explore different task scenarios and their potential impacts on revenue cycle performance.

The process 1600 concludes with the generation of GUI info 1620, which transforms the worklist information into a format suitable for presentation on the user device 1602. This step may involve creating various visual elements, such as charts, graphs, or interactive widgets, to effectively communicate the assigned tasks and their priorities. In some implementations, the GUI info 1620 might be customized based on user roles or preferences, presenting different levels of detail or focusing on specific aspects of the worklist depending on the intended user. For example, a billing team supervisor might see a high-level overview of team assignments and performance metrics, while individual billers would receive detailed task lists and relevant claim information. In some implementations, the GUI info 1620 could incorporate augmented reality elements, allowing users to interact with virtual representations of assigned claims in their physical environment.

By implementing this comprehensive process for assigning and tracking medical billing claims, the AI system can help healthcare providers optimize their revenue cycle management workflows. The combination of advanced classification capabilities, intelligent biller assignment, and user-friendly presentation of worklists enables healthcare organizations to make data-driven decisions and implement effective solutions to complex billing challenges. This approach may address the technical challenges of managing large volumes of denied claims, allowing healthcare providers to improve their claim resolution rates, reduce the time to payment, and ultimately enhance their financial performance.

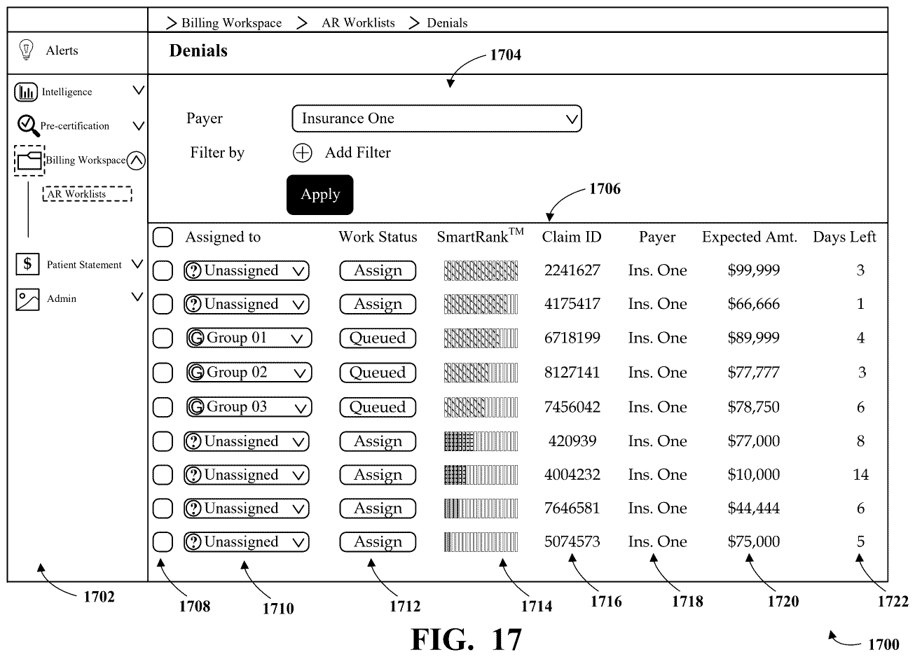

FIG. 17 illustrates a graphical user interface (GUI) 1700 for displaying and managing claim worklists according to aspects of the present disclosure. The GUI 1700 may be presented on a user device, such as a desktop computer, laptop, tablet, or smartphone, to provide healthcare revenue cycle management professionals with a comprehensive view of pending claims and associated tasks.

The GUI 1700 includes a navigation menu 1702 positioned on the left side of the interface. This navigation menu 1702 may contain options for accessing various sections of the application, such as dashboards, reports, analytics, and settings. In some implementations, the navigation menu 1702 may be collapsible to provide more screen space for data visualization. In some implementations, the navigation menu 1702 might incorporate a search functionality to quickly locate specific features or sections within the application.

A prediction type selection interface 1704 is displayed at the top of the GUI 1700, allowing users to filter and view different types of claims. This interface may include dropdown menus, radio buttons, or toggles to select various prediction categories, such as high-risk denials, potential underpayments, or claims approaching timely filing deadlines. In some aspects, the prediction type selection interface 1704 may utilize machine learning algorithms to suggest relevant prediction types based on the user's role, historical usage patterns, or current system status.

The central component of the GUI 1700 is the worklist 1706, which displays claim information in a tabular format with multiple columns. This worklist may be dynamically generated based on the selected prediction type and other filtering criteria. In some implementations, the worklist 1706 may incorporate infinite scrolling or pagination to handle large volumes of claims efficiently. In some implementations, the system may employ virtual scrolling techniques to render only the visible portion of the list, improving performance for users with limited hardware resources.

A checkbox column 1708 is included in the worklist, allowing for claim selection. This feature may enable bulk actions on selected claims, such as assigning multiple claims to a specific biller or generating batch appeal letters. In some aspects, the checkbox column 1708 may incorporate smart selection capabilities, such as automatically selecting all claims that meet certain criteria or suggesting optimal groupings of claims for efficient processing.

The assignment column 1710 shows the assignment status of each claim, indicating whether a claim has been assigned to a specific biller or team for processing. This column may use color-coding or icons to quickly convey assignment status. In some implementations, the assignment column 1710 may incorporate drag-and-drop functionality, allowing managers to easily reassign claims between team members. In some implementations, the system may employ an AI-driven assignment recommendation engine to suggest optimal claim assignments based on biller expertise and workload.

A status column 1712 indicates the current state of each claim, such as “Assigned,” “In Progress,” or “Pending Review.” This column may use a standardized set of status labels to ensure consistency across the organization. In some aspects, the status column 1712 may incorporate automated status updates based on actions taken within the system or integrations with external payer portals. The system may also employ machine learning algorithms to predict the likelihood of status changes and flag claims that may require immediate attention.

The worklist 1706 includes a ranking indicator column 1714, which provides a visual representation of claim priority or ranking. This may be displayed as a numerical score, color-coded indicator, or custom icon set. In some implementations, the ranking indicator may be dynamically calculated based on multiple factors such as claim value, age, likelihood of denial, and potential impact on cash flow. The system may utilize advanced machine learning models to continuously refine and improve the ranking algorithm based on historical outcomes and user feedback.

A claim identifier column 1716 displays unique claim identification numbers for each entry in the worklist. These identifiers may be hyperlinked to provide quick access to detailed claim information. In some aspects, the claim identifier column 1716 may incorporate intelligent search and filtering capabilities, allowing users to quickly locate specific claims or groups of related claims. The system may also employ natural language processing techniques to enable users to search for claims using conversational queries.

The payer column 1718 shows the insurance provider associated with each claim. This information may be useful for understanding the context of each claim and applying payer-specific processing rules. In some implementations, the payer column 1718 may incorporate tooltips or expandable sections to provide quick access to payer contact information, policy details, or historical performance metrics. The system may also utilize machine learning algorithms to identify patterns in payer behavior and flag claims that may require special handling based on historical trends.

An amount column 1720 presents the expected reimbursement amount for each claim. This financial information may be helpful for prioritizing work and assessing the potential impact of claim resolutions. In some aspects, the amount column 1720 may incorporate predictive analytics to display not just the billed amount, but also the expected payout based on historical data and payer behavior. The system may employ sophisticated machine learning models to continuously refine these predictions, taking into account factors such as contract terms, denial patterns, and recent policy changes.

The timing column 1722 shows the number of days left for processing each claim, which may be useful for managing timely filing deadlines and prioritizing urgent cases. This column may use color-coding or visual indicators to highlight claims approaching deadlines. In some implementations, the timing column 1722 may incorporate predictive analytics to estimate the expected time to resolution for each claim based on historical data and current workload. The system may also employ reinforcement learning techniques to optimize workflow recommendations, balancing the urgency of approaching deadlines with the likelihood of successful resolution.

By presenting this comprehensive set of information in a structured and intuitive format, the GUI 1700 may enable healthcare revenue cycle management professionals to efficiently prioritize, assign, and manage claims. The integration of machine learning and predictive analytics throughout the interface may allow for data-driven decision-making and proactive issue resolution, potentially leading to improved financial performance and reduced revenue leakage for healthcare providers.

To further describe some implementations in greater detail, reference is next made to examples of techniques which may be performed by or using an AI system as described herein. FIG. 18 is a flowchart of an example of a technique 1800 associated with providing alerts associated with healthcare financial workflows. The technique 1800 can be executed using computing devices, such as the systems, hardware, and software described with respect to FIGS. 1A-17. The technique 1800 can be performed, for example, by executing a machine-readable program or other computer-executable instructions, such as routines, instructions, programs, or other code. The steps, or operations, of the technique 1800, or another technique, method, process, or algorithm described in connection with the implementations disclosed herein can be implemented directly in hardware, firmware, software executed by hardware, circuitry, or a combination thereof.

For simplicity of explanation, the technique 1800 is depicted and described herein as a series of steps or operations. However, the steps or operations of the technique 1800 can occur in various orders and/or concurrently. In some implementations, other steps or operations not presented and described herein may be used. Furthermore, not all illustrated steps or operations may be required to implement a technique in accordance with the disclosed subject matter.

At 1802, the technique 1800 includes ingesting data from a set of disparate computer-based data sources. In some implementations, these computer-based data sources may include EHR systems, practice management systems, clearinghouse services, payer portals, and financial management systems. For example, a data ingestion pipeline (e.g., the data ingestion pipeline 414 shown in FIG. 4, which may include the communication component 110 shown in FIGS. 1A-1C) may be used to communicate with a set of computer-based data sources, which may transmit data to the communication component 110. The transmitted data may be ingested based on being received, decrypted, or decoded, among other examples. The ingested data may encompass various aspects of healthcare revenue cycle management, such as patient information, claim details, payment histories, and payer policies. In some implementations, the data sources may also include real-time patient monitoring devices or wearable health trackers to provide a more comprehensive view of patient health and potential billing implications.

At 1804, the technique 1800 may involve generating structured data for storage in a database in accordance with a unified data schema. For example, a data processing component (e.g., the data processing component 112 shown in FIGS. 1A-1C) may generate structured data for storage in a database in accordance with a unified data schema by performing, based on a first set of machine learning models, a transformation operation on the data. In some implementations, the transformation process may include data cleaning, normalization, and enrichment operations tailored to healthcare revenue cycle management. For example, the system may employ entity resolution techniques to reconcile patient and provider information across different systems, ensuring data consistency and accuracy. Some implementations may incorporate NLP capabilities to extract meaningful information from unstructured clinical notes or payer correspondence.

At 1806, the technique 1800 may include identifying an anomaly associated with a transactional workflow. For example, an ML component (e.g., the ML component 116 shown in FIGS. 1A-1C and/or the anomaly detection model 140 shown in FIG. 1D) may identify an anomaly associated with a transactional workflow by performing an anomaly detection operation on the structured data. This anomaly detection operation may be based on a second set of machine learning models. In some implementations, the transactional workflow may comprise healthcare insurance claims, claim denials, claim payments, claim reimbursements, claim submissions, or claim payment histories. The anomaly detection models may utilize techniques such as isolation forests, autoencoders, or clustering algorithms to flag potential issues like fraudulent claims, coding errors, or sudden changes in payer behavior. Some implementations may incorporate time series analysis to detect temporal anomalies, such as unexpected spikes in denial rates or changes in payment patterns over time.

At 1808, the technique 1800 may involve generating an alert event based on the identified anomaly. For example, an interface component (e.g., the interface component 118 shown in FIG. 1A and/or the application service component 130 shown in FIG. 1D) may generate an alert event based on the identified anomaly. In some implementations, this step may include automatically transforming the technical anomaly detection results into actionable information for end-users. The alert generation process may consider factors such as the type of anomaly, its potential financial impact, and historical patterns to determine the urgency and relevance of the alert. Some implementations may use natural language generation techniques to create human-readable alert descriptions that clearly communicate the issue and its implications.

At 1810, the technique 1800 may include determining an alert profile associated with a user device. For example, an interface component (e.g., the interface component 118 shown in FIG. 1A and/or the application service component 130 shown in FIG. 1D) may determine an alert profile associated with a user device. In some implementations, this step may involve tailoring the alert presentation based on user-specific or role-specific preferences. For example, the system may determine the role of a user associated with the user device and customize the alert profile accordingly. A billing specialist might receive detailed alerts about specific claims, while a financial executive might see higher-level alerts about overall revenue trends. Some implementations may incorporate machine learning to adapt the alert profile to user behavior over time, improving the relevance and effectiveness of alerts based on how users interact with and respond to them.

At 1812, the technique 1800 may involve outputting alert data based on the alert event and the alert profile. For example, an interface component (e.g., the interface component 118 shown in FIG. 1A and/or the application service component 130 shown in FIG. 1D) may determine an alert profile associated with a user device. In some implementations, the interface component may output the alert data to another component of the AI system. This alert data may be configured to cause a user interface of the user device to present a user interface element associated with the alert event. In some implementations, the user interface element may comprise a selectable option configured to cause the user interface to present information associated with the anomaly. For example, the alert data may include various elements such as the alert description, relevant metrics, suggested actions, and links to more detailed information. Some implementations may design the alert data to support interactive visualizations or augmented reality displays, allowing users to explore the underlying data and context of the alert more intuitively.

In some implementations, the technique 1800 may also include performing a data profiling operation on the received data using a third set of machine learning models. This data profiling step may involve analyzing and understanding the structure, content, and quality of incoming data from various sources. Data profiling techniques may include statistical analysis, pattern recognition, and metadata extraction to identify data types, formats, relationships, and anomalies within the input data. Some implementations may employ machine learning algorithms to automate the data profiling process, enabling the discovery of complex data patterns and interdependencies that may not be apparent through manual inspection.

The technique 1800 may further involve establishing a set of data flows associated with the set of data sources, where each data flow is associated with a different respective data schema. This step may facilitate handling the diverse data formats and structures encountered in healthcare revenue cycle management. In some implementations, the system may employ various data integration techniques, including API connections, SFTP file transfers, and database replication, to ensure a continuous flow of up-to-date information. Some implementations may incorporate blockchain technology to enhance data integrity and traceability, particularly for sensitive healthcare information.

In some implementations, the technique 1800 may include determining claim structure patterns associated with the data based on the set of data schemas. This step may involve analyzing the various data schemas to identify common patterns and structures in healthcare claims across different systems and payers. The system may then generate or update the second set of machine learning models based on these claim structure patterns. This approach may allow the anomaly detection models to adapt to the specific characteristics and nuances of the healthcare provider's claim data, potentially improving the accuracy and relevance of the detected anomalies.

The technique 1800 may also incorporate a feedback mechanism to continuously improve the AI system's performance. In some implementations, this may involve annotating the alert event with feedback metadata to generate an enriched alert. The system may receive user input from an alert user device associated with an alert user, indicating the relevance or usefulness of the alert. This feedback can be used to refine the anomaly detection models, adjust alert prioritization, and improve the overall effectiveness of the alerting system. Some implementations may employ reinforcement learning techniques to optimize the alert generation and presentation process based on user interactions and outcomes.

FIG. 19 is a flowchart of an example of a technique 1900 associated with data integration and transformation for healthcare revenue cycle management. The technique 1900 can be executed using computing devices, such as the systems, hardware, and software described with respect to FIGS. 1A-17. The technique 1900 can be performed, for example, by executing a machine-readable program or other computer-executable instructions, such as routines, instructions, programs, or other code. The steps, or operations, of the technique 1900, or another technique, method, process, or algorithm described in connection with the implementations disclosed herein can be implemented directly in hardware, firmware, software executed by hardware, circuitry, or a combination thereof.

For simplicity of explanation, the technique 1900 is depicted and described herein as a series of steps or operations. However, the steps or operations of the technique 1900 can occur in various orders and/or concurrently. Additionally, other steps or operations not presented and described herein may be used. Furthermore, not all illustrated steps or operations may be required to implement a technique in accordance with the disclosed subject matter.

At 1902, the technique 1900 may include generating first data profile content. For example, a data layer and/or a processing layer (e.g., the data layer 402 and/or the processing layer 404 shown in FIG. 4) may generate the first data profile content by performing, using an ML-based data profiling tool, an analysis on first data associated with a first data source. The first data profile content may identify first data schema content associated with the first data. The first data source may be a computer-based data source such as, for example, an EHR system, a practice management system, or a clearinghouse service. The ML-based data profiling tool may employ various data analysis techniques, such as statistical analysis, pattern recognition, and metadata extraction, to identify data types, formats, relationships, and anomalies within the input data.

At 1904, the technique 1900 may involve generating second data profile content. For example, a data layer and/or a processing layer (e.g., the data layer 402 and/or the processing layer 404 shown in FIG. 4) may generate the second data profile content by performing, using the ML-based data profiling tool, an analysis on second data associated with a second data source, wherein the second data profile content identifies second data schema content associated with the second data. Similar to the first data profile content, the second data profile content may identify second data schema content associated with the second data. The second data source may be different from the first data source, such as a payer portal or a financial management system. In some implementations, the first and second data profile content may further identify data type information associated with their respective data sources.

At 1906, the technique 1900 may include identifying schema matching content. For example, a data layer and/or a processing layer (e.g., the data layer 402 and/or the processing layer 404 shown in FIG. 4) may identify the schema matching content by performing, using an ML-based schema matching component, a schema matching operation wherein the schema matching content is associated with a match between a first schema associated with the first data schema content and a second schema associated with the second data schema content. This step may involve using NLP techniques to detect relationships between column names, descriptions, or metadata associated with the first and second data schema content. In some implementations, the machine learning component may comprise a rule engine configured to apply one or more rules to the first and second data profile content to generate an identification of data type information corresponding thereto.

At 1908, the technique 1900 may involve generating transformed data. For example, a processing layer (e.g., the processing layer 404 shown in FIG. 4) may generate the transformed data by performing, using an ML-based data transformation component and based on the schema matching content, a data transformation operation based on converting at least one of the first data or the second data from a source data format to a unified data format associated with a unified data schema. This operation may involve converting at least one of the first data or the second data from a source data format to a unified data format associated with a unified data schema. The unified data schema may be generated based on patterns associated with the first and second schemas, allowing for comprehensive analysis across previously siloed data sources.

At 1910, the technique 1900 may include storing the transformed data within a database. For example, a database component (e.g., the database component 114 shown in FIGS. 1A-1C) may store the transformed data. This step may involve using an ML-based data management component configured to manage data consistency, versioning, and quality within the database. In some implementations, the database may be part of a larger data lake architecture designed to efficiently store and manage large volumes of diverse healthcare data.

In some implementations, the technique 1900 may further include performing, using an ML-based data cleaning component, a data cleaning operation to correct anomalies in the first or second data. This operation may involve standardizing medical code formats, handling inconsistent units of measurement, or resolving conflicts between overlapping data elements. In some implementations, an ML-based completion tool may be employed to complete incomplete fields in the data, using historical trends or similarity analysis to infer missing information.

The technique 1900 may also involve performing, using an ML-based entity resolution component, an entity resolution operation to resolve entity differences or duplicate record differences between the first and second data schema information. This operation may employ fuzzy matching techniques, clustering algorithms, or deep learning models to identify and reconcile discrepancies in entity representations across different data sources.

In some implementations, the technique 1900 may include performing, using an ML-based data integration component, a data integration operation to establish data flows between the AI system and the data sources. This may involve using AI-powered APIs to facilitate seamless data exchange and synchronization across multiple databases associated with the AI system. The data integration component may also be responsible for managing the ongoing flow of data, ensuring that the AI system's database remains up-to-date with the latest information from various healthcare IT systems.

The technique 1900 may incorporate a feedback mechanism to continuously improve the AI system's performance. This may involve collecting user feedback on the accuracy and usefulness of the transformed data, which can be used to refine the data profiling, schema matching, and transformation processes. In some implementations, reinforcement learning techniques may be employed to optimize the data integration and transformation workflows based on observed outcomes and user interactions.

By leveraging AI and machine learning techniques throughout the data integration and transformation process, the technique 1900 may address the technical challenges associated with unifying disparate healthcare data sources. This approach may enable healthcare providers to gain a more comprehensive view of their revenue cycle, identify opportunities for optimization, and make data-driven decisions to improve financial performance. The resulting unified data model may serve as a foundation for advanced analytics, predictive modeling, and automated decision-making in healthcare revenue cycle management.

FIG. 20 is a flowchart of an example of a technique 2000 associated with data integration and transformation for healthcare revenue cycle management. The technique 2000 can be executed using computing devices, such as the systems, hardware, and software described with respect to FIGS. 1A-17. The technique 2000 can be performed, for example, by executing a machine-readable program or other computer-executable instructions, such as routines, instructions, programs, or other code. The steps, or operations, of the technique 2000, or another technique, method, process, or algorithm described in connection with the implementations disclosed herein can be implemented directly in hardware, firmware, software executed by hardware, circuitry, or a combination thereof.

For simplicity of explanation, the technique 2000 is depicted and described herein as a series of steps or operations. However, the steps or operations of the technique 2000 can occur in various orders and/or concurrently. Additionally, other steps or operations not presented and described herein may be used. Furthermore, not all illustrated steps or operations may be required to implement a technique in accordance with the disclosed subject matter.

At 2002, the technique 2000 may include performing data profiling operations. For example, a data layer and/or a processing layer (e.g., the data layer 402 and/or the processing layer 404 shown in FIG. 4) may be used to perform the data profiling operations. In some implementations, this step may involve using an ML-based data profiling tool of an AI system to analyze first data associated with a first data source and generate first data profile content. The first data profile content may identify first data schema content associated with the first data. In some implementations, the ML-based data profiling tool may be used to analyze second data associated with a second data source and generate second data profile content, which may identify second data schema content associated with the second data. In some aspects, the first and second data profile content may further identify data type information associated with their respective data sources. The data profiling operation may employ various data analysis techniques, such as statistical analysis, pattern recognition, and metadata extraction, to identify data types, formats, relationships, and anomalies within the input data.

At 2004, the technique 2000 may involve performing a schema matching operation on the profiled data. For example, a data layer and/or a processing layer (e.g., the data layer 402 and/or the processing layer 404 shown in FIG. 4) may perform the schema matching operation. In some implementations, this step may use an ML component of the AI system to identify schema matching content associated with a match between a first schema associated with the first data schema content and a second schema associated with the second data schema content. The machine learning component may comprise a natural language processing component configured to use natural language processing to detect relationships between column names, descriptions, or metadata associated with the first and second data schema information. In some aspects, the ML component may include a rule engine configured to apply one or more rules to the first and second data profile content to generate an identification of the data type information. The rule engine may be further configured to update these rules based on an ML clustering operation.

At 2006, the technique 2000 may include performing data standardization and cleaning operations on the matched data. For example, a data layer and/or a processing layer (e.g., the data layer 402 and/or the processing layer 404 shown in FIG. 4) may perform the data standardization and cleaning operations on the matched data. In some implementations, this step may involve using an ML-based data cleaning component to correct anomalies in the first or second data. This may include standardizing medical code formats, handling inconsistent units of measurement, or resolving conflicts between overlapping data elements. In some implementations, an ML-based completion tool may be employed to complete incomplete fields in the data, using historical trends or similarity analysis to infer missing information. In some aspects, the system may implement a standardization operation to map a first medical code format associated with the first data to a second medical code format associated with the second data.

At 2008, the technique 2000 may involve performing a data transformation operation to convert the standardized data into a desired format. For example, a processing layer (e.g., the processing layer 404 shown in FIG. 4) may perform the data transformation operation. In some implementations, this step may use an ML-based data transformation component of the AI system to generate transformed data based on the schema matching content. The operation may involve converting at least one of the first data or the second data from a source data format to a unified data format associated with a unified data schema. The unified data schema may be generated based on patterns associated with the first and second schemas, allowing for comprehensive analysis across previously siloed data sources. In some aspects, the data transformation operation may incorporate advanced entity resolution techniques, using probabilistic matching algorithms to link patient records or claims data across disparate systems, even in the absence of perfect identifier matches.

At 2010, the technique 2000 may include performing an entity resolution operation on the transformed data. For example, a processing layer (e.g., the processing layer 404 shown in FIG. 4) may perform the entity resolution operation. In some implementations, this step may use an ML-based entity resolution component to resolve entity differences or duplicate record differences between the first and second data schema content. The entity resolution operation may employ fuzzy matching techniques, clustering algorithms, or deep learning models to identify and reconcile discrepancies in entity representations across different data sources. This process may be particularly important in healthcare revenue cycle management, where patient and provider information may be represented differently across various systems.

At 2012, the technique 2000 may involve storing the transformed data within a database of the AI system. For example, a database component (e.g., the database component 114 shown in FIGS. 1A-1C) may store the transformed data. In some implementations, this step may use an ML-based data management component configured to manage data consistency, versioning, and quality within the database. The database may be part of a larger data lake architecture designed to efficiently store and manage large volumes of diverse healthcare data. In some aspects, the system may implement advanced data compression techniques or utilize a combination of relational and NoSQL databases to accommodate the diverse types of data encountered in revenue cycle management.

At 2014, the technique 2000 may include performing a data integration operation to connect the stored data with other system components. For example, a database manager (e.g., the database manager 124 shown in FIGS. 1A-1C) may perform the data integration operation. In some implementations, the database manager may include an ML-based data integration component configured to establish data flows throughout the AI system. This may involve using ML-powered APIs to facilitate seamless data exchange and synchronization across multiple databases associated with the AI system. The data integration component may also be responsible for managing the ongoing flow of data, ensuring that the AI system's database remains up-to-date with the latest information from various healthcare IT systems.

At 2016, the technique 2000 may involve performing an ML training operation using the integrated data. For example, an intelligence layer (e.g., the intelligence layer 406 shown in FIG. 4) may perform the ML training operation. In some implementations, this step may use the transformed and integrated data to train or update the ML models used throughout the AI system. This may include refining the models used for anomaly detection, predictive analytics, or automated decision-making in healthcare revenue cycle management. The training process may incorporate feedback mechanisms to continuously improve the AI system's performance based on observed outcomes and user interactions.

In some implementations, the technique 2000 may also include additional steps or variations of the described steps. For example, the system may incorporate blockchain technology to enhance data integrity and traceability, particularly for sensitive healthcare information. The data transformation process may also involve semantic enrichment, where additional context and meaning are added to the raw data. This may include mapping local codes to standardized terminologies, inferring missing information based on available data, and linking related data elements across different sources.

The technique 2000 may also implement advanced privacy and security measures throughout the data integration and transformation process. This may include techniques for data anonymization, encryption, and access control to ensure compliance with healthcare data privacy regulations such as HIPAA. In some aspects, the system may employ differential privacy techniques to allow for meaningful analysis of healthcare data while protecting individual patient privacy.

By leveraging AI and machine learning techniques throughout the data integration and transformation process, the technique 2000 may address the technical challenges associated with unifying disparate healthcare data sources. This approach may enable healthcare providers to gain a more comprehensive view of their revenue cycle, identify opportunities for optimization, and make data-driven decisions to improve financial performance. The resulting unified data model may serve as a foundation for advanced analytics, predictive modeling, and automated decision-making in healthcare revenue cycle management.

FIG. 21 is a flowchart of an example of a technique 2100 associated with generating and using predictive models for revenue cycle metrics. The technique 2100 can be executed using computing devices, such as the systems, hardware, and software described with respect to FIGS. 1A-17. The technique 2100 can be performed, for example, by executing a machine-readable program or other computer-executable instructions, such as routines, instructions, programs, or other code. The steps, or operations, of the technique 2100, or another technique, method, process, or algorithm described in connection with the implementations disclosed herein can be implemented directly in hardware, firmware, software executed by hardware, circuitry, or a combination thereof.

For simplicity of explanation, the technique 2100 is depicted and described herein as a series of steps or operations. However, the steps or operations of the technique 2100 can occur in various orders and/or concurrently. Additionally, other steps or operations not presented and described herein may be used. Furthermore, not all illustrated steps or operations may be required to implement a technique in accordance with the disclosed subject matter.

At 2102, the technique 2100 may include obtaining, from a database, a first set of data associated with an entity. For example, a data ingestion pipeline (e.g., the data ingestion pipeline 414 shown in FIG. 4) may receive the first set of data. In some implementations, this first set of data may comprise various types of healthcare-related information, such as patient demographic information, claim information, claim denial information, provider information, or payer information. The database may be part of a larger data lake architecture designed to efficiently store and manage large volumes of diverse healthcare data. In some implementations, the first set of data may also include information from real-time patient monitoring devices or wearable health trackers to provide a more comprehensive view of patient health and potential billing implications.

At 2104, the technique 2100 may involve generating, by a model generator, a machine learning model based on the first set of data. For example, an intelligence layer (e.g., the intelligence layer 406 shown in FIG. 4) may include a model generator (e.g., a software component configured to generate an ML model, select an ML model, or modify an ML model). In some implementations, the ML model may include at least one of a logistic regression model, a random forest model, a gradient boosting machine model, a neural network model, or a support vector machine model. The choice of model may depend on factors such as the specific prediction task, the nature of the available data, and the desired balance between model interpretability and predictive performance. In some implementations, the model generator may employ automated machine learning (AutoML) techniques to systematically evaluate multiple model types and select the best performing one based on predefined performance metrics.

At 2106, the technique 2100 may include generating a predictive model by training the machine learning model using at least a portion of the first set of data. For example, an ML training component (e.g., the ML training component 138 shown in FIG. 1C) may train the ML model, using at least a portion of the first set of data, to predict metrics associated with operations cycles based on data from disparate data sources. In some implementations, this step may involve performing pre-processing operations on the data, such as data cleaning, feature engineering, or normalization. The feature engineering operation may identify a set of features associated with the first set of data, which may include attributes such as claim amount, medical codes, payer type, provider type, patient demographics, or statistics associated with historical metrics corresponding to the predicted metric. In some implementations, the training process may incorporate techniques to address class imbalance, such as using a loss function based on weighted revenue event classes or performing resampling operations on minority revenue event classes.

At 2108, the technique 2100 may involve determining, based on a second set of data and using the predictive model, a predicted metric associated with an operation cycle corresponding to the entity. For example, an intelligence layer (e.g., the intelligence layer 406 shown in FIG. 4) may include the predictive model, which may predict the metric associated with the operation cycle. In some implementations, this step may be triggered by receiving an application programming interface (API) call indicative of the second set of data. The predicted metric may comprise various aspects of healthcare revenue cycle management, such as a predicted medical claim denial, a predicted revenue, a predicted payout associated with a medical claim, or an expected allowed amount associated with a medical claim. In some implementations, the system may generate multiple predicted metrics simultaneously, providing a more comprehensive analysis of the revenue cycle.

At 2110, the technique 2100 may include outputting prediction data indicative of the predicted metric. For example, an interface component and/or a communication component (e.g., the interface component 118 and/or the communication component 110 shown in FIG. 1) may output the prediction data. In some implementations, this prediction data may be configured to cause a user interface of a user device to present a user interface element associated with the predicted metric. This user interface element may include interactive visualizations, detailed metric descriptions, or links to relevant supporting documentation. In some implementations, the prediction data may be provided to a mitigation engine configured to perform, based on the prediction data, a mitigation operation such as a recommendation operation or a robotic process automation operation.

In some implementations, the technique 2100 may also include additional steps or variations of the described steps. For example, the system may incorporate blockchain technology to enhance data integrity and traceability, particularly for sensitive healthcare information. The data transformation process may also involve semantic enrichment, where additional context and meaning are added to the raw data. This may include mapping local codes to standardized terminologies, inferring missing information based on available data, and linking related data elements across different sources.

The technique 2100 may also implement advanced privacy and security measures throughout the data processing and prediction workflow. This may include techniques for data anonymization, encryption, and access control to ensure compliance with healthcare data privacy regulations such as HIPAA. In some aspects, the system may employ differential privacy techniques to allow for meaningful analysis of healthcare data while protecting individual patient privacy.

To continuously improve the performance of the predictive model, the technique 2100 may incorporate a feedback mechanism. In some implementations, this may involve retraining the predictive model based on feedback information received from users or derived from observed outcomes. This feedback loop may allow the system to adapt to changing patterns in healthcare billing and payer behavior, ensuring that the predictions remain accurate and relevant over time.

The technique 2100 may also leverage network effects by analyzing data across multiple healthcare providers. For example, if a particular payer begins denying claims for a specific procedure code more frequently, the system may detect this trend across its network of users and adjust its predictions accordingly for all affected providers. This approach may allow the prediction engine to identify and respond to industry-wide trends more quickly than traditional, siloed systems.

By leveraging advanced machine learning techniques throughout the revenue cycle prediction workflow, the technique 2100 may address the technical challenges of forecasting financial outcomes in a complex and dynamic healthcare environment. This approach may enable healthcare providers to gain more accurate insights into their future revenue, identify potential issues before they occur, and make data-driven decisions to optimize their financial performance. The combination of sophisticated data processing, predictive modeling, and actionable outputs may provide a comprehensive solution for managing the complexities of healthcare financial workflows.

FIG. 22 is a flowchart of an example of a technique 2200 associated with processing negative revenue cycle events in healthcare financial workflows. The technique 2200 can be executed using computing devices, such as the systems, hardware, and software described with respect to FIGS. 1A-17. The technique 2200 can be performed, for example, by executing a machine-readable program or other computer-executable instructions, such as routines, instructions, programs, or other code. The steps, or operations, of the technique 2200, or another technique, method, process, or algorithm described in connection with the implementations disclosed herein can be implemented directly in hardware, firmware, software executed by hardware, circuitry, or a combination thereof.

For simplicity of explanation, the technique 2200 is depicted and described herein as a series of steps or operations. However, the steps or operations of the technique 2200 can occur in various orders and/or concurrently. Additionally, other steps or operations not presented and described herein may be used. Furthermore, not all illustrated steps or operations may be required to implement a technique in accordance with the disclosed subject matter.

At 2202, the technique 2200 may include obtaining a first dataset associated with a negative operation cycle event type. For example, a data ingestion pipeline (e.g., the data ingestion pipeline 414 shown in FIG. 4) may receive the first dataset. The negative operation cycle event may be a negative workflow cycle event such as, for example, a negative revenue cycle event, a negative claim billing cycle event, or the like. In some implementations, this dataset may be received from various healthcare information technology systems, such as EHRs, practice management systems, or claims processing platforms. The negative revenue cycle event may comprise, for example, a medical claim denial, a delayed payment, or a coding error that negatively impacts the financial processes of a healthcare provider. In some implementations, the dataset may also include information from real-time patient monitoring devices or wearable health trackers to provide a more comprehensive view of the circumstances surrounding the negative revenue cycle event.

At 2204, the technique 2200 may include determining, via a first set of machine learning components, a set of causation identifiers corresponding to the negative operation cycle event. For example, a data processing layer (e.g., the data processing layer 404 shown in FIG. 4) may utilize a first set of ML components to identify the set of causation identifiers. In some implementations, this step may utilize NLP techniques to analyze and interpret human language within healthcare workflows. For example, the first set of ML components may include NLP models designed to extract causation information from unstructured text data, such as denial reason descriptions or patient notes. The causation information may be assigned appropriate causation identifiers, which may be assigned according to any number of different types of quantification schemes configured to assign a number or short text string to a concept or larger text string. These models may employ techniques like named entity recognition, sentiment analysis, and topic modeling to convert free-text information into structured, analyzable data. In some implementations, the causation identifiers may be determined using a combination of rule-based systems and machine learning models to identify patterns in historical data that correlate with specific types of negative revenue cycle events.

At 2206, the technique 2200 may include determining, via a machine learning classification component, a set of classifications. The set of classifications may include a classification of each causation identifier of the set of causation identifiers. For example, a data processing layer and/or an intelligence layer (e.g., the data processing layer 404 and/or the intelligence layer 406 shown in FIG. 4) may determine the set of classifications. In some implementations, this classification operation may involve categorizing the negative operation cycle event into predefined classes, such as “lack of medical necessity,” “incorrect coding,” or “missing documentation.” The second set of machine learning components may employ various techniques, including supervised learning algorithms trained on labeled historical data, or unsupervised learning methods such as clustering when dealing with unlabeled data. In some implementations, the classification operation may utilize ensemble methods, combining multiple models to improve overall classification accuracy and robustness.

At 2208, the technique 2200 may involve training a second set of machine learning components to classify negative operation cycle events having the negative operation cycle event type according to the set of classifications. For example, an ML training component (e.g., the ML training component 138 shown in FIG. 1C) may train the second set of ML components to classify negative operation cycle events. In some implementations, the ML training component may use any number of different types of ML training techniques as described herein. Training may include retraining such as, for example, dynamic retraining that may be automatically performed by the ML training component based on a periodic schedule or occurrence of a retraining trigger event.

At 2210, the technique 2200 may involve obtaining a second dataset associated with a negative operation cycle event of the negative operation cycle event type. For example, a data ingestion pipeline (e.g., the data ingestion pipeline 414 shown in FIG. 4) may receive the second dataset. At 2212, the technique 2200 may involve determining an event classification associated with the negative operation cycle event. For example, an intelligence layer (e.g., the intelligence layer 406 shown in FIG. 4) may determine an event classification, of the set of classifications, via the second set of machine learning components and based on the second dataset.

At 2214, the technique 2200 may involve determining, via an assignment engine and based on the event classification, at least one rectification operation parameter associated with a rectification operation corresponding to the negative operation cycle event. For example, an assignment engine (e.g., the biller assignment engine 1508 shown in FIG. 15) may determine, based on the event classification, at least one rectification operation parameter. In some implementations, the assignment engine may utilize user profiles (e.g., biller profiles) containing information such as biller expertise, workload, and historical performance to optimize task allocation. The rectification operation parameters may include, but are not limited to, an assigned biller, a priority level, a work status, or a timing parameter. For example, the system may assign high-priority denials related to medical necessity to billers with a strong track record in appealing similar denials. In some implementations, the assignment engine may employ reinforcement learning techniques to continuously optimize its decision-making processes based on the outcomes of previous assignments. In some implementations, the assignment engine may prioritize rectification operations. Prioritization of a rectification operation may be based on any number of factors such as a timing factor (e.g., a deadline), a workload, or an impact score. An impact score may be a score indicative of any type of quantitative or qualitative impact that delaying a rectification operation may cause. For example, an impact score may be based on a dollar amount associated with a claim denial.

At 2210, the technique 2200 may include outputting, based on the at least one rectification operation parameter, assignment content configured to cause a user interface of a user device to present a user interface element associated with the rectification operation. For example, an interface component and/or a communication component (e.g., the interface component 118 and/or the communication component 110 shown in FIG. 1) may output the assignment content. In some implementations, this step may involve generating a customized dashboard for different user roles, such as billing specialists or financial analysts. The user interface element may include interactive visualizations, detailed task descriptions, or links to relevant supporting documentation. In some implementations, the system may incorporate augmented reality features to provide immersive data exploration experiences for healthcare finance professionals.

In some implementations, the technique 2200 may further include performing a data pre-processing operation on a set of biller profiles, where the pre-processing operation comprises a biller specialization mapping operation. This step may involve using machine learning algorithms to analyze historical performance data and identify areas of expertise for each biller. The resulting biller profiles may be used to inform the assignment engine's decision-making process, potentially leading to more efficient and effective task allocation.

The technique 2200 may also incorporate a feedback mechanism to continuously improve the AI system's performance. In some implementations, this may involve collecting user feedback on the accuracy and usefulness of the assignments, which can be used to refine the classification models and assignment algorithms. The system may employ reinforcement learning techniques to optimize the assignment process based on observed outcomes and user interactions.

In some aspects, the technique 2200 may include outputting the causation information to an automated robotic process automation (RPA) system. This RPA system may be configured to perform at least a portion of the rectification operation, such as automatically generating appeal letters or correcting common coding errors. The RPA system may incorporate its own set of machine learning components, which can be trained using reinforcement learning techniques based on historical information and outcomes.

The technique 2200 may also involve integrating with existing revenue cycle management systems. In some implementations, the assignment information may be provided to these systems, allowing for seamless incorporation of the AI-driven insights into established workflows. The system may generate customized dashboards within these existing platforms, displaying not only task assignments but also performance metrics for individual billers or teams.

By leveraging advanced machine learning techniques throughout the negative revenue cycle event processing workflow, the technique 2200 may address the technical challenges of identifying, classifying, and resolving financial issues in a complex and dynamic healthcare environment. This approach may enable healthcare providers to optimize their revenue cycle management processes, reduce claim denials, and ultimately improve their financial performance. The combination of NLP, classification algorithms, intelligent task assignment, and integration with existing systems may provide a comprehensive solution for managing the complexities of healthcare financial workflows.

Some implementations include a system comprising a memory subsystem storing instructions; and processing circuitry configured to execute the instructions to cause the system to: obtain a first dataset associated with a negative operation cycle event type; determine, via a first set of machine learning components, a set of causation identifiers corresponding to the negative operation cycle event; determine, via a machine learning classification component, a set of classifications comprising a classification of each causation identifier of the set of causation identifiers; train a second set of machine learning components to classify negative operation cycle events having the negative operation cycle event type according to the set of classifications; obtain a second dataset associated with a negative operation cycle event of the negative operation cycle event type; determine, via the second set of machine learning components and based on the second dataset, an event classification, of the set of classifications, associated with the negative operation cycle event; determine, via an assignment engine and based on the event classification, at least one rectification operation parameter associated with a rectification operation corresponding to the negative operation cycle event; and output, based on the at least one rectification operation parameter, assignment content configured to cause a user interface of a user device to present a user interface element associated with the rectification operation.

In some implementations, the assignment engine comprises a third set of machine learning components, and wherein the processing circuitry is configured to execute the instructions to further cause the system to train, using a reinforcement learning training technique, the third set of machine learning components based on historical information.

In some implementations, the processing circuitry is configured to execute the instructions to further cause the system to train, using a reinforcement learning training technique, at least one of the first set of machine learning components, the second set of machine learning components, or a third set of machine learning components of the assignment engine.

In some implementations, to cause the system to output the assignment information, the processing circuitry is configured to execute the instructions to further cause the system to provide the assignment content to an operation cycle management system.

In some implementations, to cause the system to output the assignment content, the processing circuitry is configured to execute the instructions to further cause the system to output the assignment content to cause the user device to present a dashboard corresponding to the operation cycle management system, wherein the dashboard comprises the user interface element corresponding to the rectification operation.

In some implementations, the dashboard further comprises a second user interface element corresponding to at least one performance metric associated with a user assigned to perform the rectification operation.

In some implementations, the processing circuitry is configured to execute the instructions to further cause the system to perform, by a data pre-processing component, a pre-processing operation on a set of user profiles, wherein the pre-processing operation comprises a user specialization mapping operation.

Some implementations include a method comprising obtaining a first dataset associated with a negative operation cycle event type; determining, via a first set of machine learning components, a set of causation identifiers corresponding to the negative operation cycle event; determining, via a machine learning classification component, a set of classifications comprising a classification of each causation identifier of the set of causation identifiers; training a second set of machine learning components to classify negative operation cycle events having the negative operation cycle event type according to the set of classifications; obtaining a second dataset associated with a negative operation cycle event of the negative operation cycle event type; determining, via the second set of machine learning components and based on the second dataset, an event classification, of the set of classifications, associated with the negative operation cycle event; determining, via an assignment engine and based on the event classification, at least one rectification operation parameter associated with a rectification operation corresponding to the negative operation cycle event; and outputting, based on the at least one rectification operation parameter, assignment content configured to cause a user interface of a user device to present a user interface element associated with the rectification operation.

In some implementations, the first set of machine learning components include at least one natural language processing (NLP) component configured to extract causation content from unstructured text.

In some implementations, the negative operation cycle event comprises a medical claim denial, and wherein the classification is associated with a denial type.

In some implementations, the at least one rectification operation parameter comprises at least one of an assigned user, a priority level, a work status, or a timing parameter.

In some implementations, determining the at least one rectification operation parameter comprises obtaining a set of user profiles, and wherein the at least one rectification operation parameter is further generated based on the set of user profiles.

In some implementations, a user profile of the set of user profiles is indicative of at least one of a user name, a user expertise, a user workload, or a user performance.

In some implementations, the priority level is based on at least one of an expected value of resolution of the negative operation cycle event, a timeliness of resolution of the negative operation cycle event, or an impact score associated with the negative operation cycle event.

In some implementations, the at least one rectification operation parameter is associated with at least one additional rectification operation.

Some implementations include non-transitory computer readable medium storing instructions operable to cause one or more processors to perform operations comprising: obtaining a first dataset associated with a negative operation cycle event type; determining, via a first set of machine learning components, a set of causation identifiers corresponding to the negative operation cycle event; determining, via a machine learning classification component, a set of classifications comprising a classification of each causation identifier of the set of causation identifiers; training a second set of machine learning components to classify negative operation cycle events having the negative operation cycle event type according to the set of classifications; obtaining a second dataset associated with a negative operation cycle event of the negative operation cycle event type; determining, via the second set of machine learning components and based on the second dataset, an event classification, of the set of classifications, associated with the negative operation cycle event; determining, via an assignment engine and based on the event classification, at least one rectification operation parameter associated with a rectification operation corresponding to the negative operation cycle event; and outputting, based on the at least one rectification operation parameter, assignment content configured to cause a user interface of a user device to present a user interface element associated with the rectification operation.

In some implementations, performing the classification operation comprises performing a machine learning clustering operation.

In some implementations, performing the classification operation comprises performing a feature engineering operation to determine a set of features corresponding to the set of causation identifiers.

In some implementations, the operations further comprising outputting the set of causation identifiers to an automated robotic process automation system, wherein the automated robotic process automation system is configured to perform at least a portion of the rectification operation.

In some implementations, the automated robotic process automation system comprises a third set of machine learning components, the operations further comprising training, using a reinforcement learning training technique, the third set of machine learning components based on historical information.

As used herein, the term “component” is intended to be broadly construed as hardware and/or a combination of hardware and software. “Software” shall be construed broadly to mean instructions, instruction sets, code, code segments, program code, programs, subprograms, software modules, applications, software applications, software packages, routines, subroutines, objects, executables, threads of execution, procedures, and/or functions, among other examples, whether referred to as software, firmware, middleware, microcode, hardware description language, or otherwise. As used herein, a processor is implemented in hardware and/or a combination of hardware and software. It will be apparent that systems and/or methods described herein may be implemented in different forms of hardware and/or a combination of hardware and software. The actual specialized control hardware or software code used to implement these systems and/or methods is not limiting of the aspects. Thus, the operation and behavior of the systems and/or methods were described herein without reference to specific software code—it being understood that software and hardware can be designed to implement the systems and/or methods based, at least in part, on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

The adjectives “first,” “second,” “third,” and so on are used for contextual distinction between two or more of the modified nouns in connection with a discussion and are not meant to be absolute modifiers that apply only to a certain respective node throughout the entire document. For example, a component may be referred to as a “first component” in connection with one discussion and may be referred to as a “second component” in connection with another discussion, or vice versa. Reference to a component, a computing device, a server, a client, an application, an apparatus, a device, a system, a computing system, or the like may include disclosure of the computing device, server, client, application, apparatus, device, system, computing system, or the like, respectively, being a node. For example, disclosure that a computing device is configured to receive information from a server also discloses that a first node is configured to receive information from a second node. Consistent with this disclosure, once a specific example is broadened in accordance with this disclosure (e.g., a computing device is configured to receive information from a server also discloses that a first node is configured to receive information from a second node), the broader example of the narrower example may be interpreted in the reverse, but in a broad open-ended way. In the example above where a computing device being configured to receive information from a server also discloses a first node being configured to receive information from a second node, “first node” may refer to a first computing device, a first server, a first client, a first application, a first apparatus, a first device, a first system, a first computing system, or the like, configured to receive the information from a second node; and “second node” may refer to a second computing device, a second server, a second client, a second application, a second apparatus, a second device, a second system, a second computing system, or the like.

As used herein, unless explicitly stated otherwise, any term specified in the singular may include its plural version. For example, “a computer that stores data and runs software,” may include a single computer that stores data and runs software or two computers—a first computer that stores data and a second computer that runs software. Also “a computer that stores data and runs software,” may include multiple computers that together stored data and run software. At least one of the multiple computers stores data, and at least one of the multiple computers runs software.

As used herein, the term “computer-readable medium” encompasses one or more computer readable media. A computer-readable medium may include any storage unit (or multiple storage units) that store data or instructions that are readable by a processing system. A computer-readable medium may include, for example, at least one of a data repository, a data storage unit, a computer memory, a hard drive, a disk, or a random access memory. A computer-readable medium may include a single computer-readable medium or multiple computer-readable media. A computer-readable medium may be a transitory computer-readable medium or a non-transitory computer-readable medium.

As used herein, the term “memory subsystem” includes one or more memories, where each memory may be a computer-readable medium. A memory subsystem may encompass memory hardware units (e.g., a hard drive or a disk) that store data or instructions in software form. In some implementations or in addition, the memory subsystem may include data or instructions that are hard-wired into processing system.

A processor may include one or more chips, system-on-chips (SoCs), chipsets, packages, or devices that individually or collectively constitute or comprise a processing system. The processing system includes a processor (or “processing”) circuitry in the form of one or multiple processors, microprocessors, processing units (such as central processing units (CPUs), graphics processing units (GPUs), neural processing units (NPUs) and/or digital signal processors (DSPs)), processing blocks, application-specific integrated circuits (ASIC), programmable logic devices (PLDs) (such as field programmable gate arrays (FPGAs)), or other discrete gate or transistor logic or circuitry (all of which may be generally referred to herein individually as “processors” or collectively as “the processor” or “the processor circuitry”). One or more of the processors may be individually or collectively configurable or configured to perform various functions or operations described herein. A group of processors collectively configurable or configured to perform a set of functions may include a first processor configurable or configured to perform a first function of the set and a second processor configurable or configured to perform a second function of the set, or may include the group of processors all being configured or configurable to perform the set of functions.

The processing system may further include memory circuitry in the form of one or more memory devices, memory blocks, memory elements or other discrete gate or transistor logic or circuitry, each of which may include tangible storage media such as random-access memory (RAM) or read-only memory (ROM), or combinations thereof (all of which may be generally referred to herein individually as “memories” or collectively as “the memory” or “the memory circuitry”). One or more of the memories may be coupled (for example, operatively coupled, communicatively coupled, electronically coupled, or electrically coupled) with one or more of the processors and may individually or collectively store processor-executable code (such as software) that, when executed by one or more of the processors, may configure one or more of the processors to perform various functions or operations described herein. Additionally or In some implementations, in some examples, one or more of the processors may be preconfigured to perform various functions or operations described herein without requiring configuration by software.

As used herein, the term “engine” may include software, hardware, or a combination of software and hardware. An engine may be implemented using software stored in the memory subsystem. In some implementations, an engine may be hard-wired into the processing system. In some cases, an engine includes a combination of software stored in the memory subsystem and hardware that is hard-wired into the processing system.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc. Likewise, the terms “system” or “tool” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be a device that can, for example, tangibly contain, store, communicate, or transport a program or data structure for use by or in connection with a processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. The quality of memory or media being non-transitory refers to such memory or media storing data for some period of time or otherwise based on device power or a device power cycle. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A system, comprising:
a memory subsystem storing instructions; and
processing circuitry configured to execute the instructions to cause the system to:
obtain a first dataset associated with a negative operation cycle event type;
determine, via a first set of machine learning components, a set of causation identifiers corresponding to the negative operation cycle event;
determine, via a machine learning classification component, a set of classifications comprising a classification of each causation identifier of the set of causation identifiers;
train a second set of machine learning components to classify negative operation cycle events having the negative operation cycle event type according to the set of classifications;
obtain a second dataset associated with a negative operation cycle event of the negative operation cycle event type;
determine, via the second set of machine learning components and based on the second dataset, an event classification, of the set of classifications, associated with the negative operation cycle event;
determine, via an assignment engine and based on the event classification, at least one rectification operation parameter associated with a rectification operation corresponding to the negative operation cycle event; and
output, based on the at least one rectification operation parameter, assignment content configured to cause a user interface of a user device to present a user interface element associated with the rectification operation.

2. The system of claim 1, wherein the assignment engine comprises a third set of machine learning components, and wherein the processing circuitry is configured to execute the instructions to further cause the system to train, using a reinforcement learning training technique, the third set of machine learning components based on historical information.

3. The system of claim 1, wherein the processing circuitry is configured to execute the instructions to further cause the system to train, using a reinforcement learning training technique, at least one of the first set of machine learning components, the second set of machine learning components, or a third set of machine learning components of the assignment engine.

4. The system of claim 1, wherein, to cause the system to output the assignment information, the processing circuitry is configured to execute the instructions to further cause the system to provide the assignment content to an operation cycle management system.

5. The system of claim 4, wherein, to cause the system to output the assignment content, the processing circuitry is configured to execute the instructions to further cause the system to output the assignment content to cause the user device to present a dashboard corresponding to the operation cycle management system, wherein the dashboard comprises the user interface element corresponding to the rectification operation.

6. The system of claim 5, wherein the dashboard further comprises a second user interface element corresponding to at least one performance metric associated with a user assigned to perform the rectification operation.

7. The system of claim 1, wherein the processing circuitry is configured to execute the instructions to further cause the system to perform, by a data pre-processing component, a pre-processing operation on a set of user profiles, wherein the pre-processing operation comprises a user specialization mapping operation.

8. A method, comprising:
obtaining a first dataset associated with a negative operation cycle event type;
determining, via a first set of machine learning components, a set of causation identifiers corresponding to the negative operation cycle event;
determining, via a machine learning classification component, a set of classifications comprising a classification of each causation identifier of the set of causation identifiers;
training a second set of machine learning components to classify negative operation cycle events having the negative operation cycle event type according to the set of classifications;
obtaining a second dataset associated with a negative operation cycle event of the negative operation cycle event type;
determining, via the second set of machine learning components and based on the second dataset, an event classification, of the set of classifications, associated with the negative operation cycle event;
determining, via an assignment engine and based on the event classification, at least one rectification operation parameter associated with a rectification operation corresponding to the negative operation cycle event; and
outputting, based on the at least one rectification operation parameter, assignment content configured to cause a user interface of a user device to present a user interface element associated with the rectification operation.

9. The method of claim 8, wherein the first set of machine learning components include at least one natural language processing (NLP) component configured to extract causation content from unstructured text.

10. The method of claim 8, wherein the negative operation cycle event comprises a medical claim denial, and wherein the classification is associated with a denial type.

11. The method of claim 8, wherein the at least one rectification operation parameter comprises at least one of an assigned user, a priority level, a work status, or a timing parameter.

12. The method of claim 11, wherein determining the at least one rectification operation parameter comprises obtaining a set of user profiles, and wherein the at least one rectification operation parameter is further generated based on the set of user profiles.

13. The method of claim 12, wherein a user profile of the set of user profiles is indicative of at least one of a user name, a user expertise, a user workload, or a user performance.

14. The method of claim 11, wherein the priority level is based on at least one of an expected value of resolution of the negative operation cycle event, a timeliness of resolution of the negative operation cycle event, or an impact score associated with the negative operation cycle event.

15. The method of claim 8, wherein the at least one rectification operation parameter is associated with at least one additional rectification operation.

16. A non-transitory computer readable medium storing instructions operable to cause one or more processors to perform operations comprising:
obtaining a first dataset associated with a negative operation cycle event type; determining, via a first set of machine learning components, a set of causation identifiers corresponding to the negative operation cycle event;
determining, via a machine learning classification component, a set of classifications comprising a classification of each causation identifier of the set of causation identifiers;
training a second set of machine learning components to classify negative operation cycle events having the negative operation cycle event type according to the set of classifications;
obtaining a second dataset associated with a negative operation cycle event of the negative operation cycle event type;
determining, via the second set of machine learning components and based on the second dataset, an event classification, of the set of classifications, associated with the negative operation cycle event;
determining, via an assignment engine and based on the event classification, at least one rectification operation parameter associated with a rectification operation corresponding to the negative operation cycle event; and
outputting, based on the at least one rectification operation parameter, assignment content configured to cause a user interface of a user device to present a user interface element associated with the rectification operation.

17. The non-transitory computer readable medium of claim 16, wherein performing the classification operation comprises performing a machine learning clustering operation.

18. The non-transitory computer readable medium of claim 16, wherein performing the classification operation comprises performing a feature engineering operation to determine a set of features corresponding to the set of causation identifiers.

19. The non-transitory computer readable medium of claim 16, the operations further comprising outputting the set of causation identifiers to an automated robotic process automation system, wherein the automated robotic process automation system is configured to perform at least a portion of the rectification operation.

20. The non-transitory computer readable medium of claim 19, wherein the automated robotic process automation system comprises a third set of machine learning components, the operations further comprising training, using a reinforcement learning training technique, the third set of machine learning components based on historical information.
